(19) United States
(12) Patent Application Publication
(10) Pub. No.: US 2022/0062308 A1
(43) Pub. Date: Mar. 3, 2022

(54) METHODS AND COMPOSITIONS FOR TREATING ROSACEA

(71) Applicant: Hovione Scientia Limited, Cork (IE)

(72) Inventors: Carla Maria dos Santos Vozone, East Windsor, NJ (US); Mohammad Salman, East Windsor, NJ (US); George Nathaniel Magrath, Mount Pleasant, SC (US); Courtney Rouse Smith, Gainesville, GA (US)

(21) Appl. No.: 17/412,328

(22) Filed: Aug. 26, 2021

Related U.S. Application Data
(60) Provisional application No. 63/129,726, filed on Dec. 23, 2020.
(60) Provisional application No. 63/071,028, filed on Aug. 27, 2020.

(51) Int. Cl. A61K 31/65; A61K 9/00; A61K 9/08; A61K 47/32; A61P 17/10

(52) U.S. Cl. CPC A61K 31/65; A61K 9/0014; A61P 17/10; A61K 47/32; A61K 9/08

(57) ABSTRACT

The invention provides methods, compositions, and kits containing a minocycline topical suspension, for treating rosacea.

### CROSS-REFERENCE TO RELATED APPLICATIONS

This application claims the benefit of and priority to U.S. Provisional Patent Application Ser. No. 63/071,028, filed Aug. 27, 2020, and U.S. Provisional Patent Application Ser. No. 63/129,726, filed Dec. 23, 2020; the contents of each of which are hereby incorporated by reference in their entirety.

### FIELD OF THE INVENTION

The invention provides methods, compositions, and kits containing a minocycline topical suspension, for treating rosacea.

### BACKGROUND

Rosacea is a chronic dermatologic disorder that has been reported to affect convexities of the central face (e.g., cheeks, chin, nose, and central forehead). The prevalence of rosacea is typically highest (often between 2.7% and 10%) in patients of northern European or Celtic heritage. Rosacea is generally more common among female patients, and incidence tends to peak between the ages of 30 and 50 years. Rosacea may be further characterized according to subtype, such as the subtype papulopustular rosacea. Patients with papulopustular rosacea frequently present with constant erythema and transient pustules and papules in the central facial distribution. The inflammatory papulopustular lesions in rosacea often affect both the sebaceous and hair follicles.

Topical medications have been reported for use in patients suffering from rosacea and include metronidazole, azelaic acid, sodium sulfacetamide and sulfur, erythromycin, tretinoin, and brimonidine. However, these reported topical medications may cause local irritation to the skin. ZILXI™ is a FDA approved topical foam indicated for treating inflammatory lesions of rosacea in adults. ZILXI™ contains minocycline as an active ingredient. However, ZILXI™ is flammable, is distributed as a pressurized container subject to potential puncture, and has been reported to cause various adverse side effects when administered to a patient, including erythema, flushing, dryness, and itching. See, for example, the prescribing information for ZILXI™.

Oral medications have been reported for use in patients suffering from rosacea and include tetracycline, doxycycline, metronidazole, erythromycin, and metronidazole. Systemic therapies, however, are associated with adverse events driven by the route of administration or high systemic exposure to the drug. For example, common side effects of oral doxycycline include nausea, diarrhea, upset stomach, headache, skin rash and itching; known but less common side effects include dizziness and blurred vision. The need exists for rosacea therapies that are more effective and/or have reduced adverse side effects.

Minocycline crystal forms, formulations of minocycline, and medical uses of the foregoing have been described in, for example, U.S. Pat. Nos. 8,258,327; 9,592,246; and 10,213,443. Oral administration of minocycline to human patients has been described in the American Journal of Ophthalmology (December 2012). However, oral administration of minocycline raises the risk of adverse side effects due to systemic exposure to minocycline. Exemplary adverse effects of oral minocycline are attributable to its systemic exposure include dizziness, vestibular concerns, development of autoantibodies including anti-nuclear antibody (ANA), anti-neutrophil cytoplasmic antibody (ANCA) and anti-phospholipid antibodies with or without associated clinical symptoms. More specifically, orally administered minocycline may induce hypersensitivity reactions affecting the liver, lung, kidneys, or multiple organs in the first weeks of treatment and, with long-term treatment, may cause autoimmune reactions. In addition, long-term oral administration of minocycline may induce hyperpigmentation of the skin or other organs.

The present invention addresses the need for improved therapies for rosacea and provides other related advantages.

### SUMMARY

The invention provides methods, compositions, and kits containing a minocycline topical suspension, for treating rosacea. The minocycline topical suspension is preferably topically administered to the patient's face once per day at or near bedtime. Since administering too large a dose of minocycline topical suspension can result in undesired side effects (e.g., the patient experiencing skin irritation, the patient experiencing adverse effects due to elevated blood plasma levels of minocycline (e.g., lightheadedness, dizziness, vertigo, autoimmune syndromes, renal impairment, and anaphylaxis), and/or the patient temporarily experiencing a pronounced yellow hue to their skin due to the presence of minocycline on the surface of their skin), it is important to use a minocycline formulation that minimizes the foregoing side effects and to use a dose amount of the minocycline formulation that minimizes the foregoing side effects, while also providing sufficient minocycline to the appropriate tissue in order to achieve a therapeutic effect against rosacea. Described herein are methods for topically administering minocycline in the form of a topical suspension at the indicated dose to the face of the patient once per day to treat rosacea while minimizing the occurrence of local and systemic adverse side effects. One aspect of selecting an optimal minocycline formulation is selecting use of a topical suspension containing minocycline particles, and then further selecting a particular particle size range for the minocycline particles in such topical suspension. As described in more detail herein, there are benefits to using minocycline particles having a particular particle size range when using a minocycline topical suspension to treat medical conditions described herein. More specifically, minocycline particles having a D90 value in the range of from about 2 microns to about 8 microns, and more preferably from about 3 microns to about 5 microns, provide the advantage of achieving an optimal amount of penetration into the skin when treating medical conditions described herein, such as rosacea. Minocycline particles having a D90 value greater than about 8 microns have reduced capacity to penetrate the skin, which results in reduced ability to treat the medical disorder. On the other hand, minocycline particles having a D90 value less than about 2 microns may result in too much penetration into the skin, which results in an increased amount of minocycline in the patient's blood plasma, thereby resulting in increased adverse side effects due to systemic distribution of minocycline. The desired low amount of systemic minocycline may be characterized according to pharmacokinetic parameters, such as a minocycline blood plasma AUC_{0-24 hr} less than 0.1 h*μg/mL, a minocycline blood plasma Cₘₐₓ less than 2.45 ng/mL, and any minocycline blood plasma Tₘₐₓ in the range of 4.5 to 7.5 hours for the patient following topical administration of the minocycline topical suspension. Exemplary aspects and embodiments of the invention are described below.

One aspect of the invention provides a method of treating inflammatory lesions of rosacea. The method comprises topically administering to the face of an adult human patient in need thereof once per day at or near bedtime a dose of about 1 gram of minocycline topical suspension to treat the inflammatory lesions of rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
   wherein particles of minocycline in the topical suspension have a D90 particle size in the range of from about 3 microns to about 6 microns, and the topical suspension comprises about 3% (w/w) minocycline. The method may be further characterized according, for example, embodiments where the rosacea is papulopustular rosacea. The method may be further characterized according to, for example, embodiments where the topical suspension comprises 3% (w/w) minocycline.

Another aspect of the invention provides a method of treating rosacea. The method comprises topically administering to the face of a patient in need thereof once per day a dose of from about 0.5 g to about 1.3 g of a minocycline topical suspension to treat the rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline. The method may be further characterized according to, for example, embodiments where the rosacea is papulopustular rosacea. The method may be further characterized according to, for example, embodiments where the topical suspension comprises about 3% (w/w) minocycline.

Another aspect of the invention provides a method of treating rosacea. The method comprises topically administering to the face of a patient in need thereof once per day a dose of from about 0.5 g to about 1.3 g of a minocycline topical suspension that comprises from about 1% (w/w) to about 3% (w/w) minocycline to treat the rosacea, wherein the administering results in a minocycline blood plasma AUC_{0-24 hr} less than 0.1 h*μg/mL, a minocycline blood plasma Cₘₐₓ less than 2.45 ng/mL, and any minocycline blood plasma Tₘₐₓ is in the range of 4.5 to 7.5 hours. The method may be further characterized according to, for example, embodiments where the rosacea is papulopustular rosacea. The method may be further characterized according to, for example, embodiments where the topical suspension comprises about 3% (w/w) minocycline.

Another aspect of the invention provides a method of reducing the number of inflammatory lesions on the face of a patient suffering from rosacea. The method comprises topically administering to the face of said patient in need thereof once per day a dose of from about 0.5 g to about 1.3 g of a minocycline topical suspension to reduce the number of inflammatory lesions on the face of the patient, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline. The method may be further characterized according to, for example, embodiments where the rosacea is papulopustular rosacea. The method may be further characterized according to, for example, embodiments where the topical suspension comprises about 3% (w/w) minocycline.

Another aspect of the invention provides a method of reducing the number of inflammatory lesions on the face of a patient suffering from rosacea. The method comprises topically administering to the face of said patient in need thereof once per day a dose of from about 0.5 g to about 1.3 g of a minocycline topical suspension that comprises from about 1% (w/w) to about 3% (w/w) minocycline to reduce the number of inflammatory lesions on the face of the patient, wherein the administering results in a minocycline blood plasma AUC_{0-24 hr} less than 0.1 h*μg/mL, Cₘₐₓ less than 2.45 ng/mL, and any minocycline blood plasma Tₘₐₓ is in the range of 4.5 to 7.5 hours. The method may be further characterized according to, for example, embodiments where the rosacea is papulopustular rosacea. The method may be further characterized according to, for example, embodiments where the topical suspension comprises about 3% (w/w) minocycline.

Some or all of the foregoing methods may be further characterized according to, for example, the dose of minocycline topical suspension, time for administering the minocycline topical suspension, features of the minocycline topical suspension, patients to receive treatment, facial cleaning procedures before and after administration of the minocycline topical suspension, and results produced by the method. These and other features are described in more detail herein below.

### BRIEF DESCRIPTION OF FIGURES

FIG. 1 depicts an X-ray powder diffraction spectrum of crystalline form I of minocycline.

FIG. 2 depicts an infrared spectrum of crystalline form I of minocycline.

FIG. 3 depicts an X-ray powder diffraction spectrum of crystalline form II of minocycline.

FIG. 4 depicts an infrared spectrum of crystalline form II of minocycline.

FIG. 5 depicts an X-ray powder diffraction spectrum of crystalline form III of minocycline.

FIG. 6 depicts an infrared spectrum of crystalline form III of minocycline.

FIG. 7 depicts an index finger containing a fingertip unit of minocycline topical suspension, which is a strip of minocycline topical suspension that extends from the tip of the index finger to the first joint of the index finger.

FIG. 8 is a chart showing the disposition of subjects from the clinical trial described in Example 4.

FIG. 9 depicts, for the clinical trial described in Example 4, the mean absolute change from baseline in total inflammatory lesion count. P-values are presented for 3% HY01 at each timepoint following week 1.

FIG. 10 depicts, for the clinical trial described in Example 4, the proportion of subjects achieving success (2 grade reduction to “clear” or “almost clear” at week 12) on the Investigator Global Assessment.

FIG. 11 depicts, for the clinical trial described in Example 4, the mean plasma minocycline concentration by time point since day 84, on a linear scale. The LLOQ (0.999 g/L) is the horizontal line at the top of the graph.

FIG. 12 is a graph showing results of a viscosity vs. shear rate analysis for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil (wherein the ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol)). Different lines on the graph correspond to different lots of polymeric hydrocarbon gelling agent and/or a repetition of the viscosity vs. shear rate analysis. Shear rate sweep testing was performed on a research rheometer (DHR2, TA Instruments) fitted with a 40 mm crosshatched plate measuring system. Following a 30 second equilibration time at 25° C., the samples were exposed to a 30 second pre-shear at a rate of 0.1 s⁻¹ followed immediately by a shear rate sweep, 0.1 s⁻¹ to 10,000 s⁻¹, logarithmically scaled, 6 points per decade of shear rate, shear applied for 30 seconds at each rate with viscosity calculated over the final 5 seconds of each step.

FIG. 13 is a graph showing results of a yield stress analysis for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil (wherein the ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol)). Different lines on the graph correspond to different lots of polymeric hydrocarbon gelling agent and/or a repetition of the yield stress analysis. Oscillation testing was performed on a research rheometer (DHR2, TA Instruments) fitted with a 40 mm 1° cone and plate measuring system. Following a 60 second equilibration time at 25° C., the samples were exposed to an oscillatory stress sweep ranging from 0.1 Pa, logarithmically scaled, 10 points per decade of stress, 1 Hz oscillation frequency. A step termination was set such that if at any point the oscillation strain exceeded 1500% the test would terminate.

FIG. 14 is a graph showing results of a normal stress analysis for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil (wherein the ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol)). Different lines on the graph correspond to different lots of polymeric hydrocarbon gelling agent and/or a repetition of the normal stress analysis. Shear rate sweep testing was performed on a research rheometer (DHR2, TA Instruments) fitted with a 40 mm crosshatched plate measuring system. Normal stress data was quantified during the shear sweep testing, as described above in connection with FIG. 12.

### DETAILED DESCRIPTION OF THE INVENTION

The invention provides methods, compositions, and kits containing a minocycline topical suspension, for treating rosacea. The minocycline topical suspension is preferably topically administered to the patient's face once per day at or near bedtime. Since administering too large a dose of minocycline topical suspension can result in undesired side effects (e.g., the patient experiencing skin irritation, the patient experiencing adverse effects due to elevated blood plasma levels of minocycline (e.g., lightheadedness, dizziness, vertigo, autoimmune syndromes, renal impairment, and anaphylaxis), and/or the patient temporarily experiencing a pronounced yellow hue to their skin due to the presence of minocycline on the surface of their skin), it is important to use a minocycline formulation that minimizes the foregoing side effects and to use a dose amount of the minocycline formulation that minimizes the foregoing side effects, while also providing sufficient minocycline to the appropriate tissue in order to achieve a therapeutic effect against rosacea. Described herein are methods for topically administering minocycline in the form of a topical suspension at the indicated dose to the face of the patient once per day to treat rosacea while minimizing the occurrence of local and systemic adverse side effects. One aspect of selecting an optimal minocycline formulation is selecting use of a topical suspension containing minocycline particles, and then further selecting a particular particle size range for the minocycline particles in such topical suspension. As described in more detail herein, there are benefits to using minocycline particles having a particular particle size range when using a minocycline topical suspension to treat medical conditions described herein. More specifically, minocycline particles having a D90 value in the range of from about 2 microns to about 8 microns, and more preferably from about 3 microns to about 5 microns, provide the advantage of achieving an optimal amount of penetration into the skin when treating medical conditions described herein, such as rosacea. Minocycline particles having a D90 value greater than about 8 microns have reduced capacity to penetrate the skin, which results in reduced ability to treat the medical disorder. On the other hand, minocycline particles having a D90 value less than about 2 microns may result in too much penetration into the skin, which results in an increased amount of minocycline in the patient's blood plasma, thereby resulting in increased adverse side effects due to systemic distribution of minocycline. The desired low amount of systemic minocycline may be characterized according to pharmacokinetic parameters, such as a minocycline blood plasma AUC_{0-24 hr} less than 0.1 h*μg/mL, a minocycline blood plasma Cₘₐₓ less than 2.45 ng/mL, and any minocycline blood plasma Tₘₐₓ in the range of 4.5 to 7.5 hours for the patient following topical administration of the minocycline topical suspension. Various aspects of the invention are set forth below in sections; however, aspects of the invention described in one particular section are not to be limited to any particular section.

### Definitions

To facilitate an understanding of the present invention, a number of terms and phrases are defined below.

As used herein, the term “minocycline” refers to a compound having the following chemical structure and tautomers thereof:

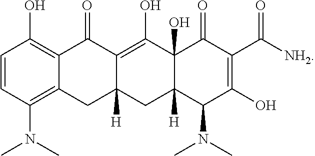 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

The terms “a,” “an” and “the” as used herein mean “one or more” and include the plural unless the context is inappropriate.

The term “about” means within 10% of the stated value. In certain embodiments, the value may be within 8%, 6%, 4%, 2%, or 1% of the stated value.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating patients suffering from rosacea by administering minocycline topical suspension to the patient. The invention also provides methods more specifically directed to treating inflammatory lesions of rosacea and directed to reducing the number of inflammatory lesions on the face of a patient suffering from rosacea. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating inflammatory lesions of rosacea. The method comprises topically administering to the face of an adult human patient in need thereof once per day at or near bedtime a dose of about 1 gram of minocycline topical suspension to treat the inflammatory lesions of rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
   wherein particles of minocycline in the topical suspension have a D90 particle size in the range of from about 3 microns to about 6 microns, and the topical suspension comprises about 3% (w/w) minocycline.

The method may be further characterized according to additional features, such as the weight percent minocycline in the topical suspension, the dose of minocycline suspension, and minocycline blood plasma pharmacokinetic parameters. For example, in certain embodiments, the topical suspension comprises 3% (w/w) minocycline. In certain embodiments, the dose is 1 g of minocycline topical suspension.

In certain embodiments, the patient's minocycline blood plasma AUC_{0-24 hr} is less than 0.1 h*μg/mL. In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL.

In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL.

In certain embodiments, any Tₘₐₓ for minocycline in the patient's blood plasma is in the range of 4.5 to 7.5 hours. The method may also be further characterized according to additional features described below.

### B. Second Method

Another aspect of the invention provides a method of treating rosacea. The method comprises topically administering to the face of a patient in need thereof once per day a dose of from about 0.5 g to about 1.3 g of a minocycline topical suspension to treat the rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
wherein particles of minocycline in the topical suspension have a D90 particle size less than 8 microns, and the topical suspension comprises from about 1% (w/w) to about 3% (w/w) minocycline.

The method may be further characterized according to additional features, such as minocycline blood plasma pharmacokinetic parameters and the rosacea symptoms treated by the method. For example, in certain embodiments, the patient's minocycline blood plasma AUC_{0-24 hr} is less than 0.1 h*μg/mL. In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL.

In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL.

In certain embodiments, any Tₘₐₓ for minocycline in the patient's blood plasma is in the range of 4.5 to 7.5 hours.

In certain embodiments, the method treats inflammatory lesions of rosacea. The method may also be further characterized according to additional features described below.

### C. Third Method

Another aspect of the invention provides a method of treating rosacea. The method comprises topically administering to the face of a patient in need thereof once per day a dose of from about 0.5 g to about 1.3 g of a minocycline topical suspension that comprises from about 1% (w/w) to about 3% (w/w) minocycline to treat the rosacea, wherein the administering results in a minocycline blood plasma AUC_{0-24 hr} less than 0.1 h*μg/mL, a minocycline blood plasma Cₘₐₓ less than 2.45 ng/mL, and any minocycline blood plasma Tₘₐₓ is in the range of 4.5 to 7.5 hours.

The method may be further characterized according to additional features, such as the rosacea symptoms treated by the method. For example, in certain embodiments, the method treats inflammatory lesions of rosacea. The method may also be further characterized according to additional features described below.

### D. Fourth Method

Another aspect of the invention provides a method of treating rosacea. The method comprises topically administering to the face of a patient in need thereof an effective amount of minocycline to treat the rosacea.

The method may be further characterized according to additional features, such as the formulation of the minocycline, minocycline blood plasma pharmacokinetic parameters, and the rosacea symptoms treated by the method. For example, in certain embodiments, the minocycline is formulated as a minocycline topical suspension. In certain embodiments, the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline.

In certain embodiments, the minocycline topical suspension comprises:
a) minocycline in a suspended form within the topical suspension;
b) a liquid medium that dissolves less than 5% (w/w) of the minocycline at room temperature after two hours; and
c) a polymeric hydrocarbon gelling agent;
   wherein particles of minocycline in the topical suspension have a D90 particle size less than 8 microns, the topical suspension comprises from about 1% (w/w) to about 3% (w/w) minocycline, and the topical suspension reduces transepidermal water loss from the patient's face. In certain embodiments, the topical suspension reduces transepidermal water loss from the patient's face by at least 10%. In certain embodiments, the topical suspension reduces transepidermal water loss from the patient's face by at least 25%. In certain embodiments, the topical suspension reduces transepidermal water loss from the patient's face by at least 1%, 2%, 3%, 4%, 5%, 6%, 7%, 8%, 9%, 10%, 15%, 20%, 25%, 30%, 35%, 40%, or 45% reduction. In certain embodiments, the polymeric hydrocarbon gelling agent comprises a thermoplastic elastomer polymer. In certain embodiments, the polymeric hydrocarbon gelling agent comprises a C₂-C₄ alkylene/C₃-C₅ alkylene/styrene copolymer.

In certain embodiments, the minocycline topical suspension comprises:
a) minocycline in a suspended form within the topical suspension;
b) a liquid medium that dissolves less than 5% (w/w) of the minocycline at room temperature after two hours; and
c) a polymeric hydrocarbon gelling agent;
   wherein particles of minocycline in the topical suspension have a D90 particle size less than 8 microns, the topical suspension comprises from about 1% (w/w) to about 3% (w/w) minocycline, and the topical suspension increases water content of skin on the patient's face. In certain embodiments, the topical suspension increases water content of skin on the patient's face by at least 5%. In certain embodiments, the topical suspension increases water content of skin on the patient's face by at least 15%. In certain embodiments, the topical suspension increases water content of skin on the patient's face by at least 1%, 2%, 3%, 4%, 5%, 6%, 7%, 8%, 9%, 10%, 15%, 20%, 25%, 30%, 35%, 40%, or 45%. In certain embodiments, the polymeric hydrocarbon gelling agent comprises a thermoplastic elastomer polymer. In certain embodiments, the polymeric hydrocarbon gelling agent comprises a C₂-C₄ alkylene/C₃-C₅ alkylene/styrene copolymer.

In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL. In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL. In certain embodiments, any Tₘₐₓ for minocycline in the patient's blood plasma is in the range of 4.5 to 7.5 hours.

In certain embodiments, the method comprises topically administering to the face of a patient in need thereof once per day a dose of from about 0.5 g to about 1.3 g of a minocycline topical suspension to treat the rosacea. In certain embodiments, the dose of minocycline topical suspension is from about 0.5 g to about 0.8 g, from about 0.5 g to about 1.0 g, from about 0.8 g to about 1.0 g, from about 0.8 g to about 1.3 g, or from about 0.8 g to about 1.0 g.

In certain embodiments, the method treats inflammatory lesions of rosacea. The method may also be further characterized according to additional features described below.

### E. Fifth Method

Another aspect of the invention provides a method of reducing the number of inflammatory lesions on the face of a patient suffering from rosacea. The method comprises topically administering to the face of said patient in need thereof once per day a dose of from about 0.5 g to about 1.3 g of a minocycline topical suspension to reduce the number of inflammatory lesions on the face of the patient, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline.

The method may be further characterized according to additional features, such as minocycline blood plasma pharmacokinetic parameters. For example, in certain embodiments, the patient's minocycline blood plasma AUC_{0-24 hr} is less than 0.1 h*μg/mL. In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL. In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL. In certain embodiments, any Tₘₐₓ for minocycline in the patient's blood plasma is in the range of 4.5 to 7.5 hours.

The method may also be further characterized according to additional features described below.

### F. Sixth Method

Another aspect of the invention provides a method of reducing the number of inflammatory lesions on the face of a patient suffering from rosacea. The method comprises topically administering to the face of said patient in need thereof once per day a dose of from about 0.5 g to about 1.3 g of a minocycline topical suspension that comprises from about 1% (w/w) to about 3% (w/w) minocycline to reduce the number of inflammatory lesions on the face of the patient, wherein the administering results in a minocycline blood plasma AUC_{0-24 hr} less than 0.1 h*μg/mL, Cₘₐₓ less than 2.45 ng/mL, and any minocycline blood plasma Tₘₐₓ is in the range of 4.5 to 7.5 hours.

The method may be further characterized according to additional features described below.

### G. Seventh Method

Another aspect of the invention provides a method of reducing the number of inflammatory lesions on the face of a patient suffering from rosacea. The method comprises topically administering to the face of said patient in need thereof an effective amount of minocycline to reduce the number of inflammatory lesions on the face of the patient.

The method may be further characterized according to additional features, such as the formulation of the minocycline and minocycline blood plasma pharmacokinetic parameters. For example, in certain embodiments, the minocycline is formulated as a minocycline topical suspension. In certain embodiments, the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline.

In certain embodiments, the minocycline topical suspension comprises:
a) minocycline in a suspended form within the topical suspension;
b) a liquid medium that dissolves less than 5% (w/w) of the minocycline at room temperature after two hours; and
c) a polymeric hydrocarbon gelling agent;
   wherein particles of minocycline in the topical suspension have a D90 particle size less than 8 microns, the topical suspension comprises from about 1% (w/w) to about 3% (w/w) minocycline, and the topical suspension reduces transepidermal water loss from the patient's face. In certain embodiments, the topical suspension reduces transepidermal water loss from the patient's face by at least 10%. In certain embodiments, the topical suspension reduces transepidermal water loss from the patient's face by at least 25%. In certain embodiments, the topical suspension reduces transepidermal water loss from the patient's face by at least 1%, 2%, 3%, 4%, 5%, 6%, 7%, 8%, 9%, 10%, 15%, 20%, 25%, 30%, 35%, 40%, or 45% reduction. In certain embodiments, the polymeric hydrocarbon gelling agent comprises a thermoplastic elastomer polymer. In certain embodiments, the polymeric hydrocarbon gelling agent comprises a C₂-C₄ alkylene/C₃-C₅ alkylene/styrene copolymer.

In certain embodiments, the minocycline topical suspension comprises:
a) minocycline in a suspended form within the topical suspension;
b) a liquid medium that dissolves less than 5% (w/w) of the minocycline at room temperature after two hours; and
c) a polymeric hydrocarbon gelling agent;
   wherein particles of minocycline in the topical suspension have a D90 particle size less than 8 microns, the topical suspension comprises from about 1% (w/w) to about 3% (w/w) minocycline, and the topical suspension increases water content of skin on the patient's face. In certain embodiments, the topical suspension increases water content of skin on the patient's face by at least 5%. In certain embodiments, the topical suspension increases water content of skin on the patient's face by at least 15%. In certain embodiments, the topical suspension increases water content of skin on the patient's face by at least 1%, 2%, 3%, 4%, 5%, 6%, 7%, 8%, 9%, 10%, 15%, 20%, 25%, 30%, 35%, 40%, or 45%. In certain embodiments, the polymeric hydrocarbon gelling agent comprises a thermoplastic elastomer polymer. In certain embodiments, the polymeric hydrocarbon gelling agent comprises a C₂-C₄ alkylene/C₃-C₅ alkylene/styrene copolymer.

In certain embodiments, the patient's minocycline blood plasma AUC_{0-24 hr} is less than 0.1 h*μg/mL In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL. In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL. In certain embodiments, any Tₘₐₓ for minocycline in the patient's blood plasma is in the range of 4.5 to 7.5 hours.

In certain embodiments, the method comprises topically administering to the face of a patient in need thereof once per day a dose of from about 0.5 g to about 1.3 g of a minocycline topical suspension. In certain embodiments, the dose of minocycline topical suspension is from about 0.5 g to about 0.8 g, from about 0.5 g to about 1.0 g, from about 0.8 g to about 1.0 g, from about 0.8 g to about 1.3 g, or from about 0.8 g to about 1.0 g.

The method may also be further characterized according to additional features described below.

### H. Additional Exemplary Features of the Second Through Seventh Therapeutic Methods

Additional exemplary features that may characterize the Second, Third, Fourth, Fifth, Sixth, and Seventh Therapeutic Methods described herein are provided below and include, for example, identity of the patient, dosing regimen used to administer the minocycline topical suspension to the patient, features of the minocycline topical suspension, and the dosing amount of minocycline topical suspension. A more thorough description of such features is provided below. The invention embraces all permutations and combinations of these features.

In certain embodiments, the patient is an adult human.

In certain embodiments, the dose is administered at or near the patient's bedtime.

### Dose of Minocycline Topical Suspension

The method may be further characterized according to the dose of minocycline topical suspension that is administered. For example, in certain embodiments, the dose is from about 0.7 g to about 1.1 g of a minocycline topical suspension. In certain embodiments, the dose is from 0.8 g to 1.1 g of a minocycline topical suspension. In certain embodiments, the dose is about 1 g of a minocycline topical suspension. In certain embodiments, the dose is 1 g of a minocycline topical suspension.

### Amount of Minocycline in the Topical Suspension

The method may be further characterized according to the amount of minocycline in the topical suspension. For example, in certain embodiments, the topical suspension comprises from about 1% (w/w) to about 3% (w/w) minocycline. In certain embodiments, the topical suspension comprises from about 1% (w/w) to about 2% (w/w) minocycline. In certain embodiments, the topical suspension comprises from about 2% (w/w) to about 3% (w/w) minocycline. In certain embodiments, the topical suspension comprises about 1% (w/w) minocycline. In certain embodiments, the topical suspension comprises 1% (w/w) minocycline. In certain embodiments, the topical suspension comprises about 3% (w/w) minocycline. In certain embodiments, the topical suspension comprises 3% (w/w) minocycline.

### Materials in the Polymeric Hydrocarbon Gelling Agent

The method may be further characterized according to the materials in the polymeric hydrocarbon gelling agent in the topical suspension. For example, in certain embodiments, the polymeric hydrocarbon gelling agent comprises an ethylene-propylene-styrene copolymer. In certain embodiments, the polymeric hydrocarbon gelling agent comprises an ethylene-propylene-styrene copolymer having a weight-average molecular weight in the range of from about 150,000 g/mol to about 250,000 g/mol. In certain embodiments, the polymeric hydrocarbon gelling agent comprises an ethylene-propylene-styrene copolymer having a weight-average molecular weight of about 200,000 g/mol.

In certain embodiments, the polymeric hydrocarbon gelling agent comprises from about 1% (w/w) to about 15% (w/w) of ethylene-propylene-styrene copolymer. In certain embodiments, the polymeric hydrocarbon gelling agent comprises from about 2.5% (w/w) to about 10% (w/w) of ethylene-propylene-styrene copolymer.

In certain embodiments, the ethylene-propylene-styrene copolymer is a copolymer formed by polymerization of isoprene and styrene monomers that is terminated by hydrogenation. In certain embodiments, the ethylene-propylene-styrene copolymer is a copolymer formed by polymerization of isoprene and styrene followed by hydrogenation.

In certain embodiments, the polymeric hydrocarbon gelling agent comprises a butylene-ethylene-styrene copolymer. In certain embodiments, the polymeric hydrocarbon gelling agent comprises a butylene-ethylene-styrene copolymer having a weight-average molecular weight in the range of from about 50,000 g/mol to about 150,000 g/mol. In certain embodiments, the polymeric hydrocarbon gelling agent comprises a butylene-ethylene-styrene copolymer having a weight-average molecular weight of about 100,000 g/mol.

In certain embodiments, the polymeric hydrocarbon gelling agent comprises from about 0.01% (w/w) to about 2.5% (w/w) of butylene-ethylene-styrene copolymer. In certain embodiments, the polymeric hydrocarbon gelling agent comprises from about 0.1% (w/w) to about 2.5% (w/w) of butylene-ethylene-styrene copolymer.

In certain embodiments, the butylene-ethylene-styrene copolymer is a copolymer formed by polymerization of 1,3-butadiene and styrene monomers that is terminated by hydrogenation.

In certain embodiments, the butylene-ethylene-styrene copolymer is a copolymer formed by polymerization of 1,3-butadiene and styrene followed by hydrogenation.

In certain embodiments, the polymeric hydrocarbon gelling agent comprises mineral oil. In certain embodiments, the polymeric hydrocarbon gelling agent comprises at least about 80% (w/w) mineral oil. In certain embodiments, the polymeric hydrocarbon gelling agent comprises at least about 90% (w/w) mineral oil.

### Particle Size of Minocycline Particles

Minocycline in the minocycline topical suspension is present in the form of particles. Methods herein may be further characterized according to the particle size of minocycline in the minocycline topical suspension. For example, in certain embodiments, the minocycline has a D90 particle size in the range of from about 3 microns to about 5 microns. In certain embodiments, the minocycline has a D90 particle size in the range of from about 3 microns to about 4 microns. In certain embodiments, minocycline particles in the minocycline topical suspension have a D90 particle size that is about 3 microns. In certain embodiments, the minocycline has a D90 particle size of about 3.5 microns. In certain embodiments, minocycline particles in the minocycline topical suspension have a D90 particle size that is about 4 microns.

In certain embodiments, minocycline particles in the minocycline topical suspension have a D90 particle size that is from 1 micron to 8 microns. In certain embodiments, minocycline particles in the minocycline topical suspension have a D90 particle size that is from 1 micron to 7 microns. In certain embodiments, minocycline particles in the minocycline topical suspension have a D90 particle size that is from 1 micron to 5 microns. In certain embodiments, minocycline particles in the minocycline topical suspension have a D90 particle size that is from 2 microns to 5 microns. In certain embodiments, minocycline particles in the minocycline topical suspension have a D90 particle size that is from 2 microns to 4 microns. In certain embodiments, minocycline particles in the minocycline topical suspension have a D90 particle size that is from 3 microns to 4 microns. In certain embodiments, minocycline particles in the minocycline topical suspension have a D90 particle size that is less than 4 microns. In certain embodiments, minocycline particles in the minocycline topical suspension have a D90 particle size that is less than 3 microns. In certain embodiments, minocycline particles in the minocycline topical suspension have a D90 particle size that is one of about 3 microns, about 4 microns, about 5 microns, about 6 microns, about 7 microns, about 8 microns, or about 9 microns, or a fractional value in between any of these values.

For example, in certain embodiments, the minocycline has a D50 particle size in the range of from about 1 microns to about 3 microns. In certain embodiments, the minocycline has a D50 particle size in the range of from about 1 microns to about 2.5 microns. In certain embodiments, the minocycline has a D50 particle size in the range of from about 1.5 microns to about 1.9 microns. In certain embodiments, the minocycline has a D50 particle size of about 1.7 microns.

In certain embodiments, minocycline particles in the minocycline topical suspension have a D50 particle size that is from about 1 micron to about 4 microns. In certain embodiments, minocycline particles in the minocycline topical suspension have a D50 particle size that is from about 1 micron to about 3 microns. In certain embodiments, minocycline particles in the minocycline topical suspension have a D50 particle size that is from about 2 micron to about 3 microns. In certain embodiments, minocycline particles in the minocycline topical suspension have a D50 particle size that is about 2.5 microns. In certain embodiments, minocycline particles in the minocycline topical suspension have a D50 particle size that is less than about 3 microns. In certain embodiments, minocycline particles in the minocycline topical suspension have a D50 particle size that is less than about 2 microns.

In certain embodiments, the minocycline has a D10 particle size in the range of from about 0.4 microns to about 1 micron. In certain embodiments, the minocycline has a D10 particle size in the range of from about 0.6 microns to about 0.8 microns. In certain embodiments, the minocycline has a D10 particle size of about 0.7 microns.

In certain embodiments, minocycline particles in the minocycline topical suspension have a D10 particle size that is from about 0.1 microns to about 3 microns. In certain embodiments, minocycline particles in the minocycline topical suspension have a D10 particle size that is from about 0.1 microns to about 2 microns. In certain embodiments, minocycline particles in the minocycline topical suspension have a D10 particle size that is from about 0.1 microns to about 1 micron. In certain embodiments, minocycline particles in the minocycline topical suspension have a D10 particle size that is from about 0.1 microns to about 1.5 microns. In certain embodiments, minocycline particles in the minocycline topical suspension have a D10 particle size that is from about 0.1 micron to about 2 microns. In certain embodiments, minocycline particles in the minocycline topical suspension have a D10 particle size that is less than 2 microns. In certain embodiments, minocycline particles in the minocycline topical suspension have a D10 particle size that is less than 1 micron.

In certain embodiments, minocycline particles in the minocycline topical suspension have a D50 particle size that is from about 1 micron to about 3 microns, and a D10 particle size that is from about 0.1 microns to about 1 micron.

### Patients for Treatment

The method may be further characterized according to the patient to be treated. For example, in certain embodiments, the patient is a human. In certain embodiments, the patient is an adult human. In certain embodiments, the patient is a pediatric human. In certain embodiments, the patient is female. In certain embodiments, the patient is male.

### I. Additional Exemplary Features of the First Through Seventh Therapeutic Methods

Additional exemplary features that may characterize the First, Second, Third, Fourth, Fifth, Sixth, and Seventh Therapeutic Methods described herein are provided below and include, for example, features of the minocycline topical suspension, dosing regimen used to administer the minocycline topical suspension to the patient. A more thorough description of such features is provided below. The invention embraces all permutations and combinations of these features.

### 1. Minocycline Topical Suspension

The method may be further characterized according to the composition of the minocycline topical suspension. For example, the method can be characterized according to the physical form of minocycline, features of the polymeric hydrocarbon gelling agent, and features of the liquid medium that dissolves less than 5% (w/w) of the minocycline at room temperature after two hours, wherein the liquid medium contains mineral oil. These are described in more detail below.

### Form of Minocycline

The method may be further characterized according to the physical form of the minocycline. For example, in certain embodiments, the minocycline is crystalline. Particular crystalline forms include Form I, Form II, and Form III. Additional details on minocycline crystalline Form I, Form II, and Form III are described herein below and in U.S. Pat. No. 8,258,327, which is hereby incorporated by reference.

Minocycline is understood to provide advantages over minocycline hydrochloride when used in a topical suspension. One advantage of minocycline is that it is less acidic than minocycline hydrochloride—the less acidic minocycline is better tolerated. That is, minocycline causes less stinging and/or burning upon administration than minocycline hydrochloride. An aqueous solution containing 1% w/v minocycline has a pH in the range of 6.0 to 6.5, whereas an aqueous solution containing 1% w/v minocycline hydrochloride has a pH in the range of 3.5 to 4.5. A further characterization of the acidity of minocycline is pKa values—minocycline has pKa values of 3.5, 7.6, and 9.2, whereas minocycline hydrochloride has pKa values of 2.8, 5.0, and 9.3. Another advantage of minocycline is that it is more lipophilic than minocycline hydrochloride. Minocycline has a log P value of 0.11, whereas minocycline hydrochloride has a log P value of 0.05.

A. Minocycline Crystalline Form I

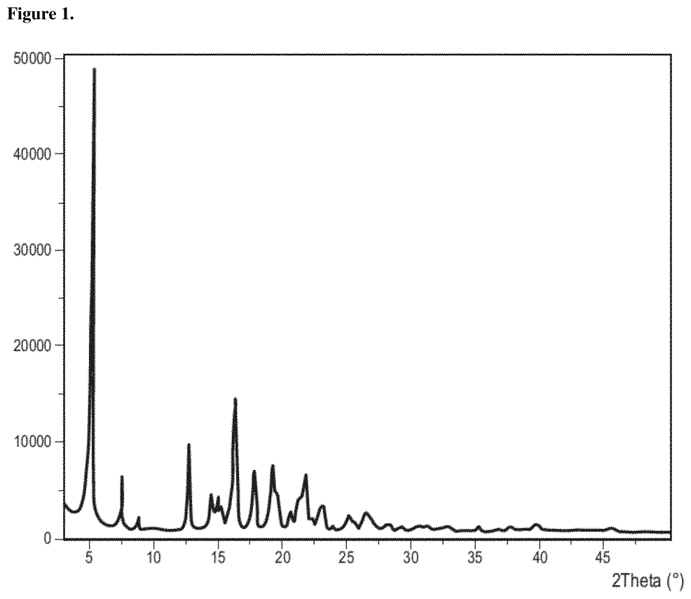

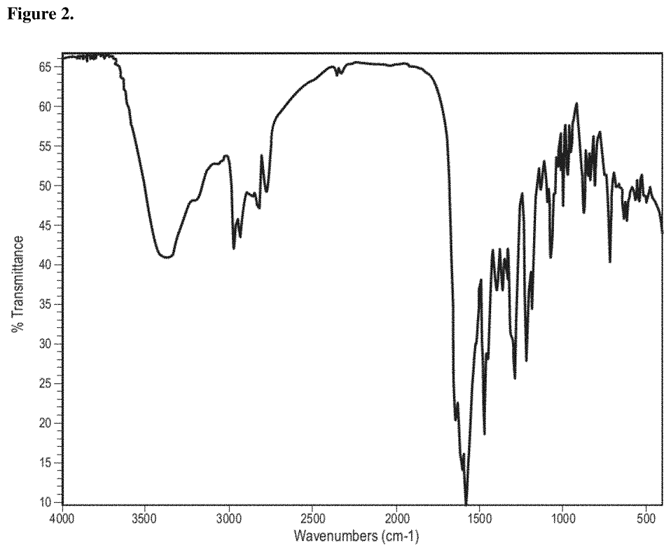

In certain embodiments, the minocycline is crystalline and characterized by an X-ray diffraction pattern having peaks at 5.2, 7.6, 8.8, 12.8, 14.5, 15.0, 15.3, 15.9, 16.4, 17.8, 19.3, 19.5, 20.7, 21.3, 21.8, 22.3, 23.1, 24.0, 25.3, 25.7 and 26.5±0.2 degrees 2θ. In certain embodiments, the minocycline is crystalline and has an X-ray diffraction pattern substantially as shown in FIG. 1. In certain embodiments, the minocycline is crystalline and characterized by an infrared spectrum having peaks at 1646, 1602, 1581, 1470, 1397, 1364, 1286, 1218, 1182, 1134, 1072, 1061, 1023, 1001, 969, 950, 874, 850, 716, 636, 620 and 545±4 cm⁻¹. In certain embodiments, the minocycline is crystalline and has an infrared spectrum substantially as shown in FIG. 2.

B. Minocycline Crystalline Form II

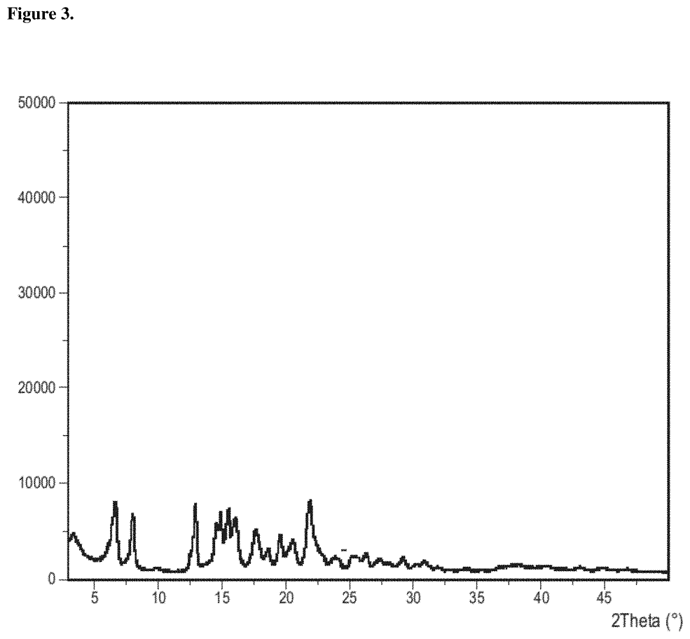

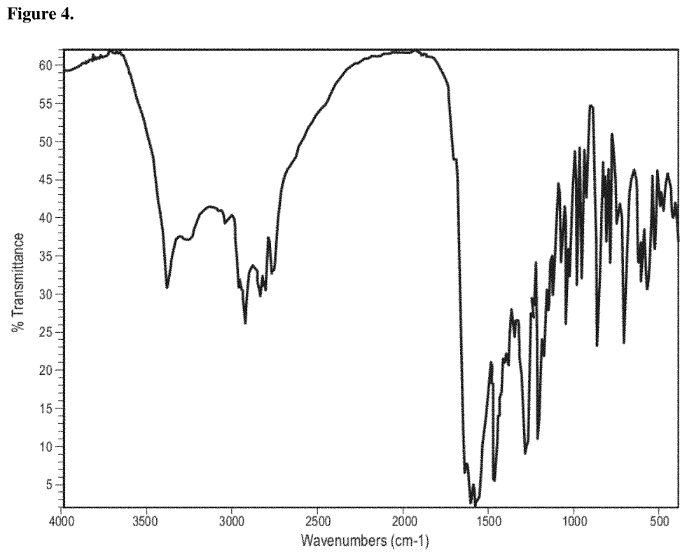

In certain embodiments, the minocycline is crystalline and characterized by an X-ray diffraction pattern having peaks at 3.4, 6.8, 8.0, 10.0, 13.0, 13.8, 22.6, and 23.9±0.2 degrees 2θ. In certain embodiments, the minocycline is crystalline and characterized by an X-ray diffraction pattern having peaks at 3.4, 6.8, 8.0, 10.0, 13.0, 13.8, 14.6, 14.9, 15.5, 16.1, 17.6, 17.8, 18.6, 19.5, 20.2, 20.6, 21.9, 22.6, 23.9, 24.2, 25.4, 26.3, 27.1, 27.5, 28.0 and 29.1±0.2 degrees 2θ. In certain embodiments, the minocycline is crystalline and has an X-ray diffraction pattern substantially as shown in FIG. 3. In certain embodiments, the minocycline is crystalline and characterized by an infrared spectrum having peaks at 1644, 1607, 1582, 1469, 1453, 1413, 1396, 1358, 1287, 1251, 1217, 1186, 1166, 1136, 1061, 999, 970, 874, 716, 621 and 585±4 cm⁻¹. In certain embodiments, the minocycline is crystalline and has an infrared spectrum substantially as shown in FIG. 4.

C. Minocycline Crystalline Form III

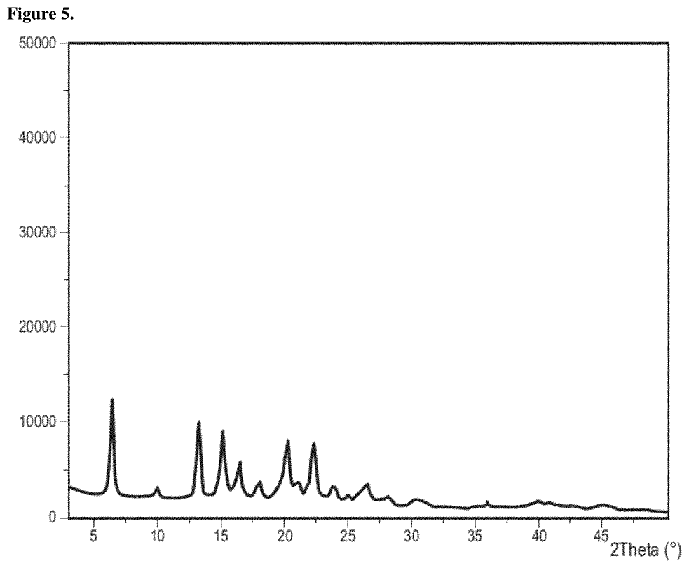

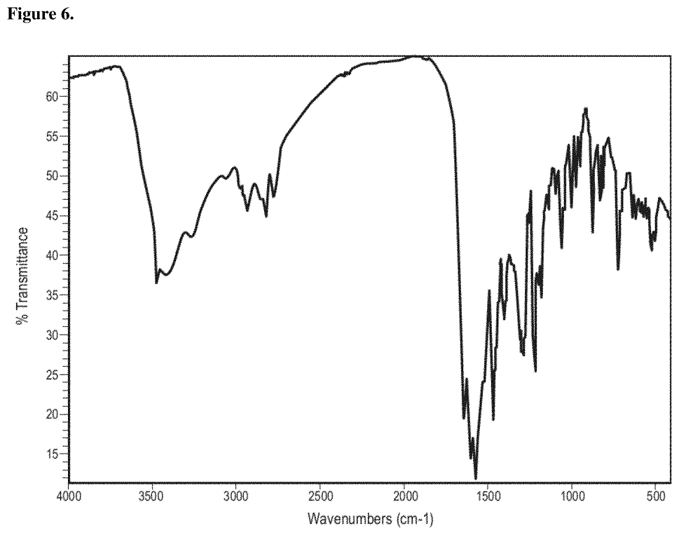

In certain embodiments, the minocycline is crystalline and characterized by an X-ray diffraction pattern having peaks at 6.5, 10.0, 13.2, 15.1, 16.5, 17.9, 19.6, 20.2, 21.1, 22.3, 23.7, 24.8, 26.4, 28.1 and 30.5±0.2 degrees 2θ. In certain embodiments, the minocycline is crystalline and has an X-ray diffraction pattern substantially as shown in FIG. 5. In certain embodiments, the minocycline is crystalline and characterized by an infrared spectrum having peaks at 1647, 1605, 1581, 1470, 1399, 1307, 1286, 1251, 1216, 1195, 1179, 1136, 1094, 1058, 1024, 1000, 973, 950, 870, 825, 806, 716, 680, 634, 615, 584, 515, 496 and 413±4 cm⁻¹. In certain embodiments, the minocycline is crystalline and has an infrared spectrum substantially as shown in FIG. 6.

### Particle Size of Minocycline Particles

Minocycline in the minocycline topical suspension is present in the form of particles. Methods herein may be further characterized according to the particle size of minocycline in the minocycline topical suspension. For example, in certain embodiments, the minocycline has a D90 particle size in the range of from about 3 microns to about 5 microns. In certain embodiments, the minocycline has a D90 particle size in the range of from about 3 microns to about 4 microns. In certain embodiments, minocycline particles in the minocycline topical suspension have a D90 particle size of about 3 microns. In certain embodiments, the minocycline has a D90 particle size of about 3.5 microns. In certain embodiments, minocycline particles in the minocycline topical suspension have a D90 particle size of about 4 microns.

For example, in certain embodiments, the minocycline has a D50 particle size in the range of from about 1 microns to about 3 microns. In certain embodiments, the minocycline has a D50 particle size in the range of from about 1 microns to about 2.5 microns. In certain embodiments, the minocycline has a D50 particle size in the range of from about 1.5 microns to about 1.9 microns. In certain embodiments, the minocycline has a D50 particle size of about 1.7 microns.

In certain embodiments, minocycline particles in the minocycline topical suspension have a D50 particle size that is from about 1 micron to about 4 microns. In certain embodiments, minocycline particles in the minocycline topical suspension have a D50 particle size that is from about 1 micron to about 3 microns. In certain embodiments, minocycline particles in the minocycline topical suspension have a D50 particle size that is from about 2 micron to about 3 microns. In certain embodiments, minocycline particles in the minocycline topical suspension have a D50 particle size that is about 2.5 microns. In certain embodiments, minocycline particles in the minocycline topical suspension have a D50 particle size that is less than about 3 microns. In certain embodiments, minocycline particles in the minocycline topical suspension have a D50 particle size that is less than about 2 microns.

In certain embodiments, the minocycline has a D10 particle size in the range of from about 0.4 microns to about 1 micron. In certain embodiments, the minocycline has a D10 particle size in the range of from about 0.6 microns to about 0.8 microns. In certain embodiments, the minocycline has a D10 particle size of about 0.7 microns.

In certain embodiments, minocycline particles in the minocycline topical suspension have a D10 particle size that is from about 0.1 microns to about 3 microns. In certain embodiments, minocycline particles in the minocycline topical suspension have a D10 particle size that is from about 0.1 microns to about 2 microns. In certain embodiments, minocycline particles in the minocycline topical suspension have a D10 particle size that is from about 0.1 microns to about 1 micron. In certain embodiments, minocycline particles in the minocycline topical suspension have a D10 particle size that is from about 0.1 microns to about 1.5 microns. In certain embodiments, minocycline particles in the minocycline topical suspension have a D10 particle size that is from about 0.1 micron to about 2 microns. In certain embodiments, minocycline particles in the minocycline topical suspension have a D10 particle size that is less than 2 microns. In certain embodiments, minocycline particles in the minocycline topical suspension have a D10 particle size that is less than 1 micron.

In certain embodiments, minocycline particles in the minocycline topical suspension have a D50 particle size that is from about 1 micron to about 3 microns, and a D10 particle size that is from about 0.1 microns to about 1 micron.

### Polymeric Hydrocarbon Gelling Agent

The method may be further characterized according to features of the polymeric hydrocarbon gelling agent in the topical suspension, such as the amount of the polymeric hydrocarbon gelling agent in the topical suspension, the presence and amount of butylated-hydroxytoluene in the polymeric hydrocarbon gelling agent, and features of the mineral oil component of the polymeric hydrocarbon gelling agent.

For example, in certain embodiments, the minocycline topical suspension comprises from about 60% (w/w) to about 80% (w/w) of the polymeric hydrocarbon gelling agent. In certain embodiments, the minocycline topical suspension comprises from about 65% (w/w) to about 75% (w/w) of the polymeric hydrocarbon gelling agent. In certain embodiments, the minocycline topical suspension comprises from about 64% (w/w) to about 72% (w/w) of the polymeric hydrocarbon gelling agent. In certain embodiments, the minocycline topical suspension comprises from about 66% (w/w) to about 70% (w/w) of the polymeric hydrocarbon gelling agent. In certain embodiments, the minocycline topical suspension comprises from about 67% (w/w) to about 71% (w/w) of the polymeric hydrocarbon gelling agent. In certain embodiments, the minocycline topical suspension comprises about 68% (w/w) of the polymeric hydrocarbon gelling agent. In certain embodiments, the minocycline topical suspension comprises about 69% (w/w) of the polymeric hydrocarbon gelling agent.

In certain embodiments, the polymeric hydrocarbon gelling agent comprises butylated-hydroxytoluene. In certain embodiments, the polymeric hydrocarbon gelling agent comprises from about 0.01% (w/w) to about 0.5% (w/w) of butylated-hydroxytoluene. In certain embodiments, the polymeric hydrocarbon gelling agent comprises butylated-hydroxytoluene in an amount less than 0.5% (w/w).

In certain embodiments, the polymeric hydrocarbon gelling agent comprises at least about 90% (w/w) mineral oil.

In certain embodiments, the mineral oil component of the polymeric hydrocarbon gelling agent has a weight-average molecular weight in the range of from about 100 g/mol to about 1,000 g/mol. In certain embodiments, the mineral oil component of the polymeric hydrocarbon gelling agent has a weight-average molecular weight in the range of from about 200 g/mol to about 700 g/mol. In certain embodiments, the mineral oil is white mineral oil.

In certain embodiments, the polymeric hydrocarbon gelling agent comprises
(a) at least 80% (w/w) mineral oil;
(b) from about 2.5% (w/w) to about 10% (w/w) of ethylene-propylene-styrene copolymer; and
(c) comprises from about 0.1% (w/w) to about 2.5% (w/w) of butylene-ethylene-styrene copolymer.

In certain embodiments, the polymeric hydrocarbon gelling agent comprises
(a) at least 80% (w/w) mineral oil having a weight-average molecular weight in the range of from about 100 g/mol to about 1,000 g/mol;
(b) from about 2.5% (w/w) to about 10% (w/w) of ethylene-propylene-styrene copolymer having a weight-average molecular weight of about 200,000 g/mol; and
(c) comprises from about 0.1% (w/w) to about 2.5% (w/w) of butylene-ethylene-styrene copolymer having a weight-average molecular weight of about 100,000 g/mol.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 13,000 to about 28,000 cps at 25° C., as sold by Calumet Specialty Products Partners, L.P. under the tradename VERSAGEL® M200. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 20,0000 cps at 25° C., as sold by Calumet Specialty Products Partners, L.P. under the tradename VERSAGEL® M200.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 47,000 to about 57,000 cps at 25° C., as sold by Calumet Specialty Products Partners, L.P. under the tradename VERSAGEL® M500. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 50,0000 cps at 25° C., as sold by Calumet Specialty Products Partners, L.P. under the tradename VERSAGEL® M500.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity in the range of from about 67,000 to about 83,000 cps at 25° C., as sold by Calumet Specialty Products Partners, L.P. under the tradename VERSAGEL® M750. In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 75,000 cps at 25° C., as sold by Calumet Specialty Products Partners, L.P. under the tradename VERSAGEL® M750.

In certain embodiments, the polymeric hydrocarbon gelling agent is a mixture of mineral oil, ethylene-propylene-styrene copolymer, butylene-ethylene-styrene copolymer, and optionally butylated-hydroxytoluene having a viscosity of about 160,0000 cps at 25° C., as sold by Calumet Specialty Products Partners, L.P. under the tradename VERSAGEL® M1600.

In certain embodiments, the polymeric hydrocarbon gelling agent comprises petrolatum, ethylene-propylene-styrene copolymer, and butylene-ethylene-styrene copolymer, as is commercially available under the tradename VERSAGEL® p series. In certain embodiments, the polymeric hydrocarbon gelling agent comprises petrolatum, ethylene-propylene-styrene copolymer, and butylene-ethylene-styrene copolymer.

In certain embodiments, the polymeric hydrocarbon gelling agent comprises USP grade white oil, ethylene-propylene-styrene copolymer, and butylene-ethylene-styrene copolymer, as is commercially available under the tradename VERSAGEL® r series. In certain embodiments, the polymeric hydrocarbon gelling agent comprises USP grade white oil, ethylene-propylene-styrene copolymer, and butylene-ethylene-styrene copolymer.

In certain embodiments, the polymeric hydrocarbon gelling agent comprises ethylene-propylene-styrene copolymer, butylene-ethylene-styrene copolymer, and dibutyl lauroyl glutamide, as is commercially available under the tradename VERSAGEL® s series. In certain embodiments, the polymeric hydrocarbon gelling agent comprises ethylene-propylene-styrene copolymer, butylene-ethylene-styrene copolymer, and dibutyl lauroyl glutamide. In certain embodiments, the polymeric hydrocarbon gelling agent comprises ethylene-propylene-styrene copolymer, butylene-ethylene-styrene copolymer, dibutyl lauroyl glutamide, hydrogenated polyisobutene, butyl stearate, and isostearyl alcohol.

In certain embodiments, the polymeric hydrocarbon gelling agent comprises ethylene-propylene-styrene copolymer, butylene-ethylene-styrene copolymer, and one or more of isohexadecane, isododecane, isopropylpalmitate, or hydrogenated polyisobutene, as is commercially available under the tradename VERSAGEL®. In certain embodiments, the polymeric hydrocarbon gelling agent comprises ethylene-propylene-styrene copolymer, butylene-ethylene-styrene copolymer, and one or more of isohexadecane, isododecane, isopropylpalmitate, or hydrogenated polyisobutene. In certain embodiments, the polymeric hydrocarbon gelling agent comprises ethylene-propylene-styrene copolymer, butylene-ethylene-styrene copolymer, and one of isohexadecane, isododecane, isopropylpalmitate, or hydrogenated polyisobutene.

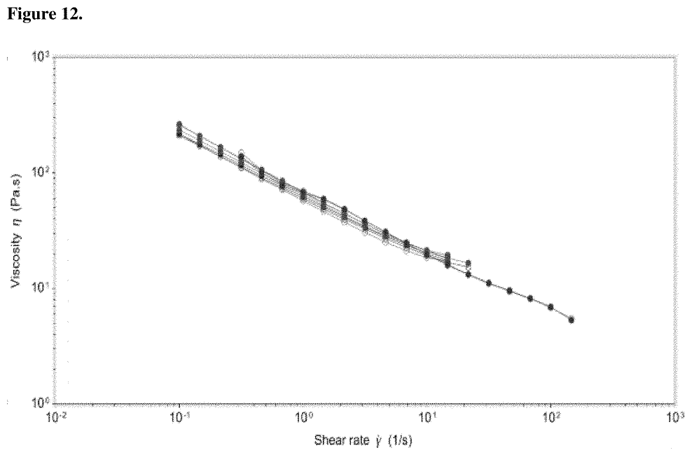

In certain embodiments, the polymeric hydrocarbon gelling agent may be further characterized according to its physical properties, such as (i) performance in a shear stress vs. shear rate analysis, (ii) yield stress analysis, and/or (iii) normal stress analysis. Results of an exemplary shear stress vs. shear rate analysis are displayed in FIG. 12 for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol). In certain embodiments, the polymeric hydrocarbon gelling agent displays substantially the same performance in shear stress vs. shear rate analysis as illustrated in FIG. 12.

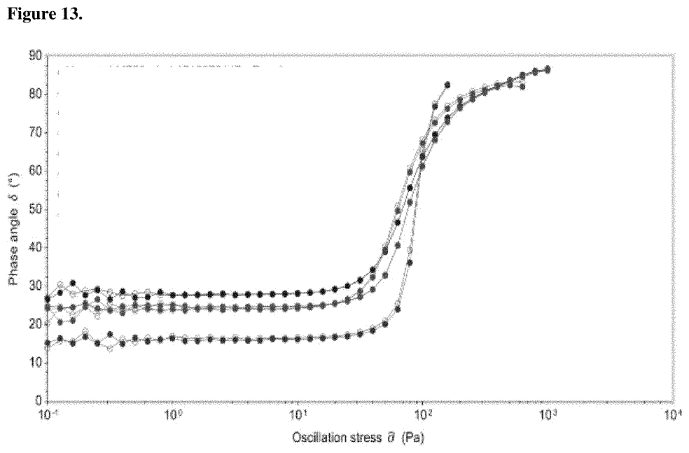

Results of an exemplary yield stress analysis are displayed in FIG. 13 for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol). In certain embodiments, the polymeric hydrocarbon gelling agent displays substantially the same performance in yield stress analysis as illustrated in FIG. 13.

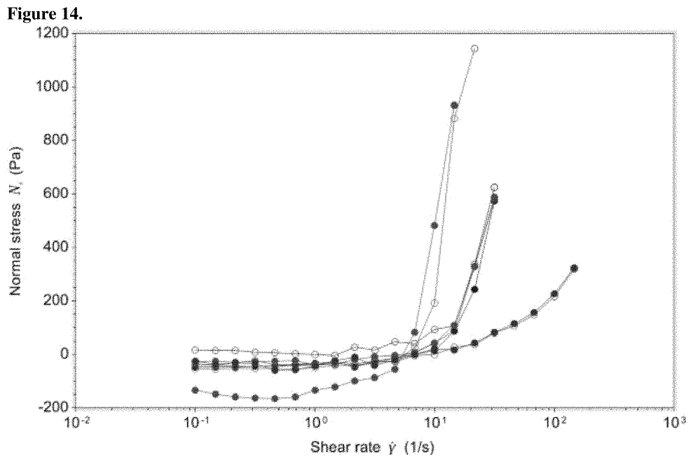

Results of an exemplary normal stress analysis are displayed in FIG. 14 for the polymeric hydrocarbon gelling agent sold commercially under the tradename VERSAGEL® M-750, which is a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol). In certain embodiments, the polymeric hydrocarbon gelling agent displays substantially the same performance in normal stress analysis as illustrated in FIG. 14.

### Liquid Medium

The method may be further characterized according to the amount of liquid medium that dissolves less than 5% (w/w) of the minocycline at room temperature after two hours. For example, in certain embodiments, the minocycline topical suspension comprises from about 20% (w/w) to about 40% (w/w) of the liquid medium that dissolves less than 5% (w/w) of the minocycline at room temperature after two hours, wherein the liquid medium contains mineral oil. In certain embodiments, the minocycline topical suspension comprises from about 25% (w/w) to about 35% (w/w) of the liquid medium that dissolves less than 5% (w/w) of the minocycline at room temperature after two hours, wherein the liquid medium contains mineral oil. In certain embodiments, the minocycline topical suspension comprises from about 27% (w/w) to about 31% (w/w) of the liquid medium that dissolves less than 5% (w/w) of the minocycline at room temperature after two hours, wherein the liquid medium contains mineral oil. In certain embodiments, the minocycline topical suspension comprises from about 28% (w/w) to about 32% (w/w) of the liquid medium that dissolves less than 5% (w/w) of the minocycline at room temperature after two hours, wherein the liquid medium contains mineral oil. In certain embodiments, the minocycline topical suspension comprises about 29% (w/w) of the liquid medium that dissolves less than 5% (w/w) of the minocycline at room temperature after two hours, wherein the liquid medium contains mineral oil. In certain embodiments, the minocycline topical suspension comprises about 30% (w/w) of the liquid medium that dissolves less than 5% (w/w) of the minocycline at room temperature after two hours, wherein the liquid medium contains mineral oil.

In certain embodiments, the liquid medium that dissolves less than 5% (w/w) of the minocycline at room temperature after two hours comprises at least 90% (w/w) mineral oil. In certain embodiments, the liquid medium that dissolves less than 5% (w/w) of the minocycline at room temperature after two hours comprises at least 99% (w/w) mineral oil. In certain embodiments, the liquid medium that dissolves less than 5% (w/w) of the minocycline at room temperature after two hours is mineral oil.

In certain embodiments, the mineral oil has a weight-average molecular weight in the range of from about 100 g/mol to about 1,000 g/mol. In certain embodiments, the mineral oil has a weight-average molecular weight in the range of from about 200 g/mol to about 700 g/mol. In certain embodiments, the mineral oil has a weight-average molecular weight in the range of from about 230 g/mol to about 700 g/mol.

In certain embodiments, the mineral oil has a weight-average molecular weight in the range of from about 200 g/mol to about 500 g/mol. In certain embodiments, the mineral oil has a weight-average molecular weight in the range of from about 300 g/mol to about 600 g/mol. In certain embodiments, the mineral oil has a weight-average molecular weight in the range of from about 400 g/mol to about 700 g/mol. In certain embodiments, the mineral oil has a weight-average molecular weight in the range of from about 400 g/mol to about 500 g/mol. In certain embodiments, the mineral oil has a weight-average molecular weight in the range of from about 440 g/mol to about 465 g/mol.

In certain embodiments, the mineral oil has a molecular weight in the range of from about 440 g/mol to about 465 g/mol. In certain embodiments, the mineral oil has a molecular weight of about 452 g/mol.

In certain embodiments, the mineral oil has a viscosity greater than 34.5 centistokes when measured at 40° C. In certain embodiments, the mineral oil has a viscosity in the range of from about 34.5 centistokes to about 150 centistokes when measured at 40° C. In certain embodiments, the mineral oil has a viscosity in the range of from about 34.5 centistokes to about 50 centistokes, from about 50 centistokes to about 75 centistokes, from about 75 centistokes to about 100 centistokes, from about 100 centistokes to about 125 centistokes, from about 125 centistokes to about 150 centistokes, or from about 34.5 centistokes to about 100 centistokes when measured at 40° C.

In certain embodiments, the mineral oil has a specific gravity of from about 0.845 to about 0.905. In certain embodiments, the mineral oil has a specific gravity of from about 0.8 to about 0.95. In certain embodiments, the mineral oil has a specific gravity of from about 0.8 to about 0.9. In certain embodiments, the mineral oil has a specific gravity of from about 0.84 to about 0.91. In certain embodiments, the mineral oil has a specific gravity of from about 0.845 to about 0.905, when measured at 20° C. In certain embodiments, the mineral oil has a specific gravity of from about 0.8 to about 0.95, when measured at 20° C. In certain embodiments, the mineral oil has a specific gravity of from about 0.8 to about 0.9, when measured at 20° C. In certain embodiments, the mineral oil has a specific gravity of from about 0.84 to about 0.91, when measured at 20° C. In certain embodiments, the mineral oil has a density of about 0.83 g/mL.

In certain embodiments, the mineral oil corresponds to the mineral oil identified by CAS registry number 8042-47-5.

In certain embodiments, the mineral oil has a viscosity less than 34.5 centistokes when measured at 40° C. In certain embodiments, the mineral oil has a viscosity less than 33.5 centistokes when measured at 40° C. In certain embodiments, the mineral oil has a viscosity in the range of from about 1 centistoke to about 34.4 centistokes when measured at 40° C. In certain embodiments, the mineral oil has a viscosity in the range of from about 1 centistoke to about 10 centistokes, from about 10 centistokes to about 20 centistokes, from about 20 centistokes to about 30 centistokes, or from about 25 centistokes to about 34.4 centistokes, when measured at 40° C.

In certain embodiments, said liquid medium dissolves less than 4% (w/w) of the minocycline at room temperature after two hours. In certain embodiments, said liquid medium dissolves less than 3% (w/w) of the minocycline at room temperature after two hours. In certain embodiments, said liquid medium dissolves less than 2% (w/w) of the minocycline at room temperature after two hours. In certain embodiments, said liquid medium dissolves less than 1% (w/w) of the minocycline at room temperature after two hours. In certain embodiments, said liquid medium dissolves less than 0.1% (w/w) of the minocycline at room temperature after two hours. In certain embodiments, said liquid medium dissolves from about 0.01% (w/w) to about 4% (w/w) of the minocycline at room temperature after two hours. In certain embodiments, said liquid medium dissolves from about 0.1% (w/w) to about 4% (w/w) of the minocycline at room temperature after two hours. In certain embodiments, said liquid medium dissolves from about 0.1% (w/w) to about 3% (w/w) of the minocycline at room temperature after two hours. In certain embodiments, said liquid medium dissolves from about 0.1% (w/w) to about 2% (w/w) of the minocycline at room temperature after two hours. In certain embodiments, said liquid medium dissolves from about 0.1% (w/w) to about 1% (w/w) of the minocycline at room temperature after two hours. In certain embodiments, said liquid medium dissolves from about 0.5% (w/w) to about 2% (w/w) of the minocycline at room temperature after two hours. In certain embodiments, said liquid medium dissolves from about 0.5% (w/w) to about 1% (w/w) of the minocycline at room temperature after two hours.

In certain embodiments, the liquid medium is characterized by dissolving less than about 4% (w/w) of minocycline when held at room temperature for two hours. In certain embodiments, the liquid medium is characterized by dissolving less than about 3% (w/w) of minocycline when held at room temperature for two hours. In certain embodiments, the liquid medium is characterized by dissolving less than about 2% (w/w) of minocycline when held at room temperature for two hours. In certain embodiments, the liquid medium is characterized by dissolving less than about 1% (w/w) of minocycline when held at room temperature for two hours.

In certain embodiments, the liquid medium is characterized by dissolving from about 0.01% (w/w) to about 4% (w/w) minocycline when held at room temperature for two hours. In certain embodiments, the liquid medium is characterized by dissolving from about 0.1% (w/w) to about 4% (w/w) minocycline when held at room temperature for two hours. In certain embodiments, the liquid medium is characterized by dissolving from about 0.1% (w/w) to about 3% (w/w) minocycline when held at room temperature for two hours. In certain embodiments, the liquid medium is characterized by dissolving from about 0.1% (w/w) to about 2% (w/w) minocycline when held at room temperature for two hours. In certain embodiments, the liquid medium is characterized by dissolving from about 0.1% (w/w) to about 1% (w/w) minocycline when held at room temperature for two hours. In certain embodiments, the liquid medium is characterized by dissolving from about 0.5% (w/w) to about 2% (w/w) minocycline when held at room temperature for two hours. In certain embodiments, the liquid medium is characterized by dissolving less about 4%, 3%, 2%, 1%, or 0.1% (w/w) minocycline when held at room temperature for two hours.

### Stability of Minocycline Topical Suspension

The method may be further characterized according to the stability of the minocycline topical suspension used. For example, in certain embodiments, the minocycline topical suspension contains less than 4% (w/w) of 4-epi-minocycline when the minocycline topical suspension is stored for 1 month at room temperature. In certain embodiments, the minocycline topical suspension contains less than 3%, 2%, or 1% (w/w) of 4-epi-minocycline when the minocycline topical suspension is stored for 1 month at room temperature. In certain embodiments, the minocycline topical suspension contains less than 3%, 2%, or 1% (w/w) of 4-epi-minocycline when the minocycline topical suspension is stored at 25° C.±2° C. under 60%±5% relative humidity for 36 months. In certain embodiments, the minocycline topical suspension contains less than 1% (w/w) of 4-epi-minocycline when the minocycline topical suspension is stored at 25° C.±2° C. under 60%±5% relative humidity for 36 months. In certain embodiments, the minocycline topical suspension contains less than 3%, 2%, or 1% (w/w) of 4-epi-minocycline when the minocycline topical suspension is stored at 40° C.±2° C. under 75%±5% relative humidity for 6 months.

In certain embodiments, less than 1% (w/w) of minocycline in the minocycline topical suspension degrades upon storage of the minocycline topical suspension at 25° C.±2° C. under 60%±5% relative humidity for 36 months. In certain embodiments, less than 1.5% (w/w) of minocycline in the minocycline topical suspension degrades upon storage of the minocycline topical suspension at 25° C.±2° C. under 60%±5% relative humidity for 36 months. In certain embodiments, less than 2% (w/w) of minocycline in the minocycline topical suspension degrades upon storage of the minocycline topical suspension at 25° C.±2° C. under 60%±5% relative humidity for 36 months. In certain embodiments, less than 5, 4, 3, 2, 1, 0.5, or 0.1% (w/w) of minocycline in the minocycline topical suspension degrades upon storage of the minocycline topical suspension at 25° C.±2° C. under 60%±5% relative humidity for 36 months. In certain embodiments, less than 5, 4, 3, 2, 1, 0.5, or 0.1% (w/w) of minocycline in the minocycline topical suspension degrades upon storage of the minocycline topical suspension at 25° C.±2° C. under 60%±5% relative humidity for 24 months. In certain embodiments, less than 5, 4, 3, 2, 1, 0.5, or 0.1% (w/w) of minocycline in the minocycline topical suspension degrades upon storage of the minocycline topical suspension at 405° C.±2° C. under 75%±5% relative humidity for 6 months.

In certain embodiments, storage of the minocycline topical suspension at 25° C.±2° C. under 60%±5% relative humidity for 36 months results in the formulation of less than 2% (w/w) total impurities. In certain embodiments, storage of the minocycline topical suspension at 25° C.±2° C. under 60%±5% relative humidity for 36 months results in the formulation of less than 3% (w/w) total impurities. In certain embodiments, storage of the minocycline topical suspension at 25° C.±2° C. under 60%±5% relative humidity for 36 months results in the formulation of less than 8%, 7%, 6%, 5%, 4%, 3%, 2%, or 1% (w/w) total impurities. In certain embodiments, storage of the minocycline topical suspension at 25° C.±2° C. under 60%±5% relative humidity for 24 months results in the formulation of less than 8%, 7%, 6%, 5%, 4%, 3%, 2%, or 1% (w/w) total impurities. In certain embodiments, storage of the minocycline topical suspension at 40° C.±2° C. under 75%±5% relative humidity for 6 months results in the formulation of less than 8%, 7%, 6%, 5%, 4%, 3%, 2%, or 1% (w/w) total impurities.

### Sterility Level of the Minocycline Topical Suspension

The method may be further characterized according to the sterility of the minocycline topical suspension used. For example, in certain embodiments, the minocycline topical suspension has undergone sterilization, such as by exposing the minocycline topical suspension gamma or e-beam sterilization. The level of sterility of the minocycline topical suspension may be characterized, e.g., where the minocycline topical suspension has a sterility assurance level that is more sterile than 10⁻¹, 10⁻², 10⁻³, 10⁻⁴, 10⁻⁵, 10⁻⁶, 10⁻⁷, 10⁻⁸, or 10⁻⁹. In certain embodiments, the minocycline topical suspension has a sterility assurance level of from about 10⁻¹ to 10⁻³, about 10⁻³ to about 10⁻⁴, about 10⁻⁴ to about 10⁻⁵, about 10⁻⁵ to about 10⁻⁶, or about 10⁻⁶ to about 10⁻⁷, or a sterility assurance level that is more sterile than 10⁻⁷. In certain embodiments, the minocycline topical suspension has a sterility assurance level of about 10⁻⁶.

### Non-Newtonian Physical Properties

The minocycline topical suspension desirably displays non-Newtonian physical properties. That is, in certain embodiments, the minocycline topical suspension is a non-Newtonian fluid. Accordingly, in certain embodiments, the minocycline topical suspension undergoes a reduction in viscosity due to mechanical force imposed on the minocycline topical suspension.

Viscosity of the topical minocycline suspension can be measured at different shear rates. For example, in certain embodiments, at a shear rate of 6 (1/s), the minocycline topical suspension has a viscosity in the range of from about 1,000 cP to about 45,000 cP, from about 3,000 cP to about 30,000 cP, from about 3,000 cP to about 25,000 cP, from about 3,000 cP to about 20,000 cP, from about 3,000 cP to about 15,000 cP, from about 5,000 cP to about 30,000 cP, from about 5,000 cP to about 25,000 cP, from about 5,000 cP to about 20,000 cP, from about 5,000 cP to about 15,000 cP, from about 6,000 cP to about 20,000 cP. In certain embodiments, at a shear rate of 6 (1/s), the minocycline topical suspension has a viscosity in the range of from about 7,000 cP to about 15,000 cP. In certain embodiments, at a shear rate of 6 (1/s), the minocycline topical suspension has a viscosity in the range of from about 25 Pa·s to about 35 Pa·s. In certain embodiments, at a shear rate of 6 (1/s), the minocycline topical suspension has a viscosity in the range of from about 28 Pa·s to about 32 Pa·s. In certain embodiments, at a shear rate of 6 (1/s), the minocycline topical suspension has a viscosity of about 30 Pa·s.

In certain embodiments, at a shear rate of 6 (1/s), the minocycline topical suspension has a viscosity in the range of from about 25 Pa·s to about 35 Pa·s. In certain embodiments, at a shear rate of 6 (1/s), the minocycline topical suspension has a viscosity in the range of from about 28 Pa·s to about 32 Pa·s. In certain embodiments, at a shear rate of 6 (1/s), the minocycline topical suspension has a viscosity of about 30 Pa·s.

The topical minocycline suspension can also be characterized according to Oscillatory Stress Sweep, Oscillatory Frequency Sweep, Yield stress, Complex Modulus, and Loss Modulus. Additionally, the topical minocycline suspension can also be characterized according to normal stress test, which monitors the normal stress exhibited at a range of shear rates.

In certain embodiments, the minocycline topical suspension has a zero shear viscosity in the range of from about 250 Pa·s to about 350 Pa·s. In certain embodiments, the minocycline topical suspension has a zero shear viscosity in the range of from about 275 Pa·s to about 315 Pa·s. In certain embodiments, the minocycline topical suspension has a zero shear viscosity in the range of from about 290 Pa·s to about 300 Pa·s. In certain embodiments, the minocycline topical suspension has a zero shear viscosity of about 295 Pa·s. In certain embodiments, the minocycline topical suspension has a zero shear viscosity in the range of from about 300 Pa·s to about 350 Pa·s. In certain embodiments, the minocycline topical suspension has a zero shear viscosity in the range of from about 320 Pa·s to about 335 Pa·s. In certain embodiments, the minocycline topical suspension has a zero shear viscosity of about 328 Pa·s

In certain embodiments, the minocycline topical suspension has a complex modulus plateau in the range of from about 130 Pa to about 150 Pa. In certain embodiments, the minocycline topical suspension has a complex modulus plateau in the range of from about 135 Pa to about 145 Pa. In certain embodiments, the minocycline topical suspension has a complex modulus plateau in the range of from about 138 Pa to about 141 Pa. In certain embodiments, the minocycline topical suspension has a phase angle plateau in the range of from about 20 degrees to about 30 degrees. In certain embodiments, the minocycline topical suspension has a phase angle plateau in the range of from about 24 degrees to about 28 degrees. In certain embodiments, the minocycline topical suspension has a phase angle plateau in the range of from about 25 degrees to about 27 degrees. In certain embodiments, the minocycline topical suspension has a yield stress in the range of from about 15 Pa to about 25 Pa. In certain embodiments, the minocycline topical suspension has a yield stress in the range of from about 18 Pa to about 24 Pa. In certain embodiments, the minocycline topical suspension has a yield stress in the range of from about 20 Pa to about 22 Pa.

### Exemplary More Specific Embodiments

The disclosure provides the following additional specific embodiments. Accordingly, in certain embodiments, the minocycline topical suspension comprises:
(a) about 1% (w/w) to about 3% (w/w) minocycline;
(b) about 60% (w/w) to about 80% (w/w) of the polymeric hydrocarbon gelling agent; and
(c) about 20% (w/w) to about 40% (w/w) of the liquid medium that dissolves less than 5% (w/w) of the minocycline at room temperature after two hours, wherein the liquid medium contains mineral oil.

In certain embodiments, the minocycline topical suspension comprises:
(a) about 1% (w/w) minocycline;
(b) about 69% (w/w) of the polymeric hydrocarbon gelling agent; and
(c) about 30% (w/w) of the liquid medium that dissolves less than 5% (w/w) of the minocycline at room temperature after two hours, wherein the liquid medium contains mineral oil.

The disclosure provides the following additional specific embodiments. Accordingly, in certain embodiments, the minocycline topical suspension comprises:
(a) about 3% (w/w) minocycline;
(b) about 63% (w/w) to about 72% (w/w) of the polymeric hydrocarbon gelling agent; and
(c) about 25% (w/w) to about 33% (w/w) of the liquid medium that dissolves less than 5% (w/w) of the minocycline at room temperature after two hours, wherein the liquid medium contains mineral oil.

In certain embodiments, the minocycline topical suspension comprises:
(a) about 3% (w/w) minocycline;
(b) about 68% (w/w) of the polymeric hydrocarbon gelling agent; and
(c) about 29% (w/w) of the liquid medium that dissolves less than 5% (w/w) of the minocycline at room temperature after two hours, wherein the liquid medium contains mineral oil.

### 2. Dosing and Cleansing Considerations

The method may be further characterized according to, for example, timing for the administration of the minocycline suspension to the patient, the location to which the dosage is administered on the patient, and cleansing procedures to remove remaining minocycline topical suspension.

### Timing for Administration

The method may be further characterized according to the timing for administration of the minocycline topical suspension. For example, in certain embodiments, the minocycline topical suspension is topically administered to the face of a patient near the patient's bedtime. In certain embodiments, the minocycline topical suspension is topically administered to the face of a patient from about 0.5 hours to 1 hour before the patient's bedtime. In certain embodiments, the minocycline topical suspension is topically administered to the face of a patient at about 0.5 hours before the patient's bedtime. In certain embodiments, the minocycline topical suspension is topically administered to the face of a patient at about 1 hour before the patient's bedtime.

In certain embodiments, the patient's face is washed prior to topically administering the minocycline topical suspension. In certain embodiments, the patient's face is washed using mild soap and then dried prior to topically administering the minocycline topical suspension.

### Location for Administration

The method may be further characterized according to the location for administration of the minocycline topical suspension. For example, in certain embodiments, the minocycline topical suspension is topically administered to the face of a patient by applying an aliquot of minocycline topical suspension to each of the left check, right cheek, left temple, right temple, and chin of the patient's face, and then massaging the minocycline topical suspension across the patient's face.

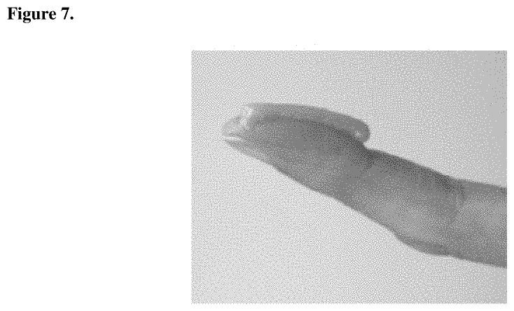

In certain embodiments, the patient applies a fingertip unit of minocycline topical suspension to each of the left check, right cheek, left temple, right temple, and chin of the patient's face, and then massages the minocycline topical suspension across the patient's face. The fingertip unit is a strip of minocycline topical suspension that extends from the tip of the index finger to the first joint of the index finger, as illustrated in FIG. 7.

In certain embodiments, contact between the minocycline topical suspension and the patient's mouth, eyes, and nostrils is avoided.

### Removing Minocycline Topical Suspension

The method may be further characterized according to the cleansing procedures used to remove remaining minocycline topical suspension. For example, in certain embodiments, the patient refrains from topically removing the administered minocycline topical suspension for at least four hours. In certain embodiments, the patient refrains from topically removing the administered minocycline topical suspension for at least 2, 4, 6, 8, 10, or 12 hours.

In certain embodiments, after the patient has completed a full daily cycle of sleep, a cleanser is topically administered to the patient's face to remove remaining minocycline topical suspension. In certain embodiments, after the patient has completed a full night of sleep, a cleanser is topically administered to the patient's face in the morning to remove remaining minocycline topical suspension. In certain embodiments, a full daily cycle of sleep comprises at least 6, 7, 8, 9, 10, 11, or 12 hours of sleep.

In certain embodiments, the cleanser comprises mineral oil, paraffin, bees wax, ceresin, petroleum, or a combination thereof. In certain embodiments, the cleanser comprises mineral oil.

In certain embodiments, the cleanser is Ponds Cold Cream, Ponds Cucumber Cleanser, Beauty 360 Moisturizing Cleanser For Softer Cleaner Skin, Albolene moisturizing cleanser, Avene Cold Cream, or Bioderma Facial Cold Cream. In certain embodiments, the cleanser is Ponds Cold Cream Cleanser. In certain embodiments, the cleanser is one of the cleansers in the following table:

| Cleanser | Exemplary Ingredients |
|---|---|
| Ponds Cold Cream | Mineral Oil, Water, Beeswax, Ceresin, Sodium Borate, Fragrance, |
| | Carbomer. |
| Ponds Cucumber | Water/Agua, Mineral Oil, Isopropyl Palmitate, Glycerin, Cetyl Alcohol, |
| Cleanser | Ceteth-20, Triethanolamine, Cetearyl Alcohol, Glyceryl Stearate, |
| | Carbomer, Fragrance/Fragancia, Methylparaben, Magnesium Aluminum |
| | Silicate, Diazolidinyl Urea, Iodopropynyl Butylcarbamate, Cucumis |
| | Sativus (Cucumber) Extract. |
| Beauty 360 Moisturizing | Mineral Oil, Petrolatum, Paraffin, Ceresin, Beta-Carotene |
| Cleanser For Softer | |
| Cleaner Skin | |
| Albolene moisturizing | Mineral Oil, Petrolatum, Paraffin, Ceresin, Beta Carotene. |
| cleanser | |
| Avene Cold Cream | Mineral Oil (Paraffinum Liquidum), Avène Thermal Spring Water |
| | (Avène Aqua), Beeswax (Cera Alba), Glyceryl Stearate, Cetyl Alcohol, |
| | Bht, C20-40 Pareth-10, Caprylyl Glycol, Cetyl Phosphate, Fragrance, |
| | Polyacrylate-13, Polyisobutene, Polysorbate 20, Sodium Hydroxide, |
| | Sorbitan Isostearate, Triethanolamine, Water (Aqua) |
| Bioderma Facial Cold | Aqua/Water/Eau, Paraffinum Liquidum/Mineral Oil/Huile Minerale, |
| Cream | Glycerin, Isostearyl Isostearate, Silica Dimethyl Silylate, Cera |
| | Alba/Beeswax/Cire D'Abeille, Cetearyl Alcohol, Polyglyceryl-3 |
| | Ricinoleate, Sorbitan Isostearate, Zinc Stearate, Sodium Citrate, |
| | Capryloyl Glycine, Pentylene Glycol, Prunus Armeniaca (Apricot) Kernel |
| | Oil, Xanthan Gum, Fragrance (Parfum). |

In certain embodiments, the cleanser comprises water and one or more of cetyl alcohol, steareth-21, salicylic acid, polysorbate 60, sodium lauryl sulfate, and glycerin.

In certain embodiments, the cleanser is L'Oreal Paris Age Perfect Cream Cleanser, Burt's Bees Intense Hydration Cream Cleanser, Moisturizing Face Wash, CVS Health Deep Clarifying Cleanser, Vichy Purete Thermale 3-in-1 One Step Face Wash and Makeup Remove, Clean & Clear Deep Action Cream Cleanser, Neutrogena Deep Clean Oil-Free Daily Facial Cream Cleanser, CeraVe Foaming Facial Cleanser, Face Wash for Oil Control, Paula's Choice Gentle Touch Makeup Remover, Paula's Choice Defense Hydrating Gel to Cream, Versed Day Dissolve Cleansing, Aveeno Positively Radiant, Up and Up Oil Free Makeup Remover, Clinique liquid facial soap, Clinique take the day off make-up remover, or Allies of Skin Molecular skin Hydrating Cleanser.

In certain embodiments, the cleanser is one of the cleansers in the following table:

| Cleanser | Exemplary Ingredients |
|---|---|
| L'Oreal Paris Age | AQUA/WATER, SODIUM COCOYL GLYCINATE, COCO-BETAINE, |
| Perfect Cream | GLYCERIN, SODIUM CHLORIDE CI 77891/TITANIUM DIOXIDE, |
| Cleanser | ACRYLATES/C10-30 ALKYL ACRYLATE CROSSPOLYMER, |
| | PHENOXYETHANOL SODIUM BENZOATE. PEG-14M, GLYCOL |
| | DISTEARATE, SODIUM HYDROXIDE, PARFUM/FRAGRANCE, |
| | METHYLPARABEN, SALICYLIC ACID, POLYQUATERNIUM-39, |
| | ETHYLPARABEN, LINALOOL, HEXYL CINNAMAL, MACADAMIA |
| | TERNIFOLIA SEED OIL, SIMMONDSIA CHINENSIS SEED OIL/JOJOBA |
| | SEED OIL, ASCORBYL GLUCOSIDE. |
| Burt's Bees Intense | Water, stearic acid, glycerin, sodium lauroyl glutamate, zea mays (corn) starch, |
| Hydration Cream | ricinus communis (castor) seed oil, parfum (natural fragrance), citrus aurantium |
| Cleanser, | dulcis (orange) oil, salvia sclarea (clary) extract, lactobacillus, theobroma |
| Moisturizing Face | grandiflorum seed butter, cera alba (beeswax, cire d'abeille), cocos nucifera |
| Wash | (coconut) oil, olea europaea (olive) fruit oil, jojoba esters, hydrolyzed jojoba |
| | esters, citrus medica limonum (lemon) peel oil, citrus aurantium amara (bitter |
| | orange) peel, polymnia sonchifolia root juice, zinc oxide, alpha-glucan |
| | oligosaccharide, propanediol, maltodextrin, tocopherol, xanthan gum, glycine |
| | soja (soybean) oil, arginine, sodium lauroyl lactylate, potassium sorbate, |
| | sodium benzoate, phenoxyethanol, amyl cinnamal, citral, eugenol, |
| | hydroxycitronellal, limonene, linalool. |
| CVS Health Deep | Water, Cetyl Alcohol, PPG-15 Stearyl Ether, Gelatin, Steareth-21, Salicylic |
| Clarifying Cleanser | Acid, Polysorbate 60, Potassium Cetyl Phosphate, Fragrance, Menthol, |
| | Disodium EDTA. |
| Clean & Clear | Water, Cetyl Alcohol, PPG-15 Stearyl Ether, Salicylic Acid, Polysorbate 60, |
| Deep Action | Steareth-21, Potassium Cetyl Phosphate, Xanthan Gum, Fragrance, Menthol, |
| Cream Cleanser | Disodium EDTA. |
| Vichy Purete | Water, Paraffinum Liquidum/Mineral Oil, Isopropyl Myristate, Potassium |
| Thermale 3-in-1 | Phosphate, Carbomer, Parfum/Fragrance, Sodium Hydroxide, Dipotassium |
| One Step Face | Phosphate, Disodium Edta, Disodium Cocoamphodiacetate, Myrtrimonium |
| Wash and Makeup | Bromide, Butylene Glycol, Butyrospermum Parkii Seedcake Extract/Shea |
| Remove | Butter Seedcake Extract. |
| Neutrogena Deep | water, cetyl alcohol, ppg-15 stearyl ether, methyl gluceth-20, steareth-21, |
| Clean Oil-Free | salicylic acid, gelatin, steareth-2, polysorbate 60, potassium cetyl phosphate, |
| Daily Facial Cream | menthol, fragrance, disodium EDTA. |
| Cleanser | |
| CeraVe Foaming | Purified Water, Cocamidopropyl Hydroxysultaine, Glycerin, Sodium Lauroyl |
| Facial Cleanser, | Sarcosinate, PEG-150 Pentaerythrityl Tetrastearate and PEG-6 Caprylic/Capric |
| Face Wash for Oil | Glycerides, Niacinamide, Propylene Glycol, Sodium Methyl Cocoyl Taurate, |
| Control | Ceramide 3, Ceramide 6-II, Ceramide 1, Hyaluronic Acid, Cholesterol, Sodium |
| | Chloride, Phytosphingosine, Citric Acid, Edetate Disodium, Dihydrate, Sodium |
| | Lauroyl Lactylate, Methylparaben, Propylparaben, Carbomer, Xanthan Gum. |
| Paula's Choice | Water (Aqua), Cyclotehasiloxane (hydration), Cyclopentasiloxane (hydration), |
| Gentle Touch | Isohexadecane (texture-enhancing), Butylene Glycol (hydration, Glycerin |
| Makeup Remover | (hydration/skin replenishing). Aloe Barbadensis Leaf Juice (skin-soothing), |
| | Bisabolol (skin-soothing), Camellia Oleifera Leaf Extract (green tea |
| | extact/antioxidant), Poloxamer 184 (texture-enhancing), Sodium |
| | Dehydroacetate (texture-enhancing), Allantoin (skin-soothing) Glycyrrhetinic |
| | Acid (skin-soothing), Sodium Chloride (stabilizer), Disodium EDTA |
| | (stabilizer), Methylparaben (preservative), Benzalkonium Chloride |
| | (preservative), Benzyl Alcohol (preservative). |
| Paula's Choice | Water, Sodium Cocoyl Glycinate (cleansing agent), Cocamidopropyl |
| Defense Hydrating | Hydroxysultaine (cleansing agent), Sodium Lauroamphoacetate (cleansing |
| Gel to Cream | agent), Cocamidopropyl Betaine (cleansing/lather agent), Glycerin (skin- |
| | replenishing), Sorbeth-230 Tetraoleate (texture enhancer/emulsifier), Decyl |
| | Glucoside (cleansing agent), Pentylene Glycol (hydration), Sodium Chloride |
| | (stabilizer/texture enhancer), Disodium Cocoyl Glutamate (cleansing agent), |
| | Laurie Acid (cleansing agent), Lactic Acid (hydration), Sodium Lauroyl Oat |
| | Amino Acids (skin-soothing), Sorbitan Laurate (cleansing agent/emulsifier), |
| | Sodium Cocoyl Glutamate (cleansing agent), Disodium EDTA. (chelating |
| | agent), Aloe Barbadensis Leaf Juice (hydration), Caprylic/Capric Triglyceride |
| | (skin-replenishing), Cocoyl Proline (hydration), Sodium Citrate (pH adjuster, |
| | Camellia Sinensis Leaf Extract (antioxidant), Glycyrrhiza Glabra (Licorice) |
| | Root Extract (skin-soothing), Sodium PCA (skin-replenishing), Sodium Lactate |
| | (hydration), Arginine (amino acid/hydration), Glycine Soja (Soybean) Sterols |
| | (antioxidant/hydration), Linoleic Acid (skin-restoring), Phospholipids (skin- |
| | restoring), Aspartic Acid, PCA, Glycine, Alanine, Serine, Valine, Isoleucine, |
| | Proline, Threonine (amino acids/hydration), Potassium Citrate (chelating |
| | agent), Lactoperoxidase (hydration), Histidine, Phenylalanine (amino |
| | acids/hydration), Glucose Oxidase (hydration), Citric Acid (pH adjuster), |
| | Phenoxyethanol, Ethylhexylglycerin, Potassium Sorbate, Sodium Benzoate |
| | (preservatives). |
| Versed Day | Ethylhexyl Palmitate, Isopropyl Palmitate, Peg-20 Glyceryl Triisostearate, |
| Dissolve Cleansing | Polyethylene, Tocopheryl Acetate, Eucalyptus Globulus Leg Oil, Eugenia |
| | Caryophylius (Clove) Leaf Oil, Simmondsia Chinensis (Jojoba) Seed Oil, |
| | Persea Gratissima (Avocado) Oil, Prunus Armeniaca (Apricot) Kernel Oil, |
| | Sclerocarya Birrea Seed Oil, Sesamum Indicum (Sesame) Seed Oil, Butyl |
| | Methoxydibenzoylmethane, Caprylyl Glycol, Eugcnol, Limonene |
| Aveeno Positively | Water, Glycerin, Sodium Laureth Sulfate, Polyethylene, Peg-16 Soy Sterol, |
| Radiant | Lauryl Glucoside, Acrylates/C10-30 Alkyl Acrylate Crosspolymer, |
| | Phenoxyethanol, Glycol Distearate, Cocamidopropyl Betaine, Methylparaben, |
| | Butylene Glycol, Fragrance, Laureth-4, Tetrasodium Edta, Sodium Hydroxide |
| | Glycine Soja (Soybean) Protein |
| Up and Up Oil Free | Water, Cyclopentasiloxane, Cyclohexasiloxane, Aloe Barbadensis Leaf Juice, |
| Makeup Remover | Benzyl Alcohol, Sodium Chloride, Dipropylene. Glycol, Poloxamer 182, |
| | Allantoin, Tripotassium Edta, Benzalkonium Chloride, Glycerin, Cucumis |
| | Sativus (Cucumber) Fruit Extract, Potassium Phosphate, Dipotassium |
| | Phosphate. |
| Clinique liquid | Water\Aqua\Eau, Sodium Laureth Sulfate, Lauramidopropyl Betaine, |
| facial soap | Cocamidopropyl Hydroxysultaine, Sodium Chloride, Sodium Cocoyl |
| | Sarcosinate, Tea-Cocoyl Glutamate, Butylene Glycol, Aloe Barbadensis Leaf |
| | Juice, Peg-120 Methyl Glucose Dioleate, Sucrose, Hexylene Glycol, |
| | Polyquaternium-7, Laureth-2, Caprylyl Glycol, Sodium Sulfate, Edta, Disodium |
| | Edta, Methylchloroisothiazolinone, Methylisothiazolinone, Phenoxyethanol |
| Clinique take the | Water\Aqua\Eau, Isododecane, Isohexadecane, Hydrogenated Polyisobutene, |
| day off make-up | Peg-4 Dilaurate, Lauryl Methyl Gluceth-10 Hydroxypropyldimonium Chloride, |
| remover | Hexylene Glycol, Sodium Chloride, Potassium Phosphate, Dipotassium |
| | Phosphate, Dipotassium Edta, Phenoxyethanol |
| DHC Deep | Olea Europaea (Olive) Fruit Oil, Caprylic/Capric Triglyceride, Sorbeth-30 |
| Cleansing Oil | Tetraoleate, Pentylene Glycol, Phenoxyethanol, Tocopherol, Stearyl |
| | Glycyrrhetinate, Rosmarinus Officinalis (Rosemary) Leaf Oil. |
| Allies of Skin | Aqua (Water), Polyacrylate-1 Crosspolymer, Sodium C14-16 Olefin Sulfonate, |
| Molecular skin | Glycerin, Cocamidopropyl Hydroxysultaine, Sodium Cocoamphoacetate, |
| Hydrating Cleanser | Carthamus Tinctorius (Safflower) Seed Oil (Organic), Propanediol, Helianthus |
| | Annuus (Sunflower) Seed Oil (Organic), Leuconostoc/Radish Root Ferment |
| | Filtrate, Lactic Acid (L), Hydrolyzed Jojoba Esters, Moringa Oleifera Seed Oil |
| | (Organic), 3-O-Ethyl Ascorbic Acid, Cocodimonium Hydroxypropyl Silk |
| | Amino Acids, Hydrolyzed Silk, Camellia Sinensis (White Tea) Leaf Extract, |
| | Camellia Oleifera (Green Tea) Leaf Extract, Sodium Hyaluronate (L), Lecithin, |
| | Astaxanthin, Xanthan Gum, Citric Acid, Lauryl Glucoside, Sodium Cocoyl |
| | Glutamate, Sodium Lauryl Glucose Carboxylate, Caprylyl Glycol, O-cymen-5- |
| | OL, Polysorbate 20, Tetrasodium EDTA, Phenethyl Alcohol. |
| Angela Cagela | Butyrospermum Parkii (Shea) Butter, Caprylyl/Capryl Glucoside, |
| Power Cleansing | Caprylic/Capric Triglyceride, Sorbitan Olivate (Olive Oil), Glyceryl Stearate, |
| Balm | Cetyl Alcohol, Limnanthes Alba (Meadowfoam) Seed Oil, Borage Officinalis |
| | (Borage) Seed Oil, Tocopheryl Acetate (Vitamin E), Lecithin, Pelargonium |
| | Graveolens (Rose Geranium) Oil, Chamomilla Recutita (Matricaria) Flower |
| | Extract |

### Duration of Treatment

The method may be further characterized according to the duration of treatment. For example, in certain embodiments, for a duration of at least 4 weeks the patient receives a dose of minocycline topical suspension each day. In certain embodiments, for a duration of at least 8 weeks the patient receives a dose of minocycline topical suspension each day. In certain embodiments, for a duration of at least 12 weeks the patient receives a dose of minocycline topical suspension each day. In certain embodiments, for a duration of at least 24 weeks the patient receives a dose of minocycline topical suspension each day. In certain embodiments, for a duration of at least twelve months the patient receives a dose of minocycline topical suspension each day.

In certain embodiments, for a duration of at least 1, 2, 3, 4, 5, 6, 7, 8, 9, 10, 11, 12, 13, 14, 15, 16, 17, 18, 19, 20, 21, 22, 23, or 24 weeks the patient receives a dose of minocycline topical suspension each day.

3. Patient Populations that May Derive Particular Benefits from the Therapeutic Methods

The method may be further characterized according to the patient to be treated. For example, in certain embodiments, the patient has at least 10 inflammatory lesions on their face due to rosacea prior to starting treatment with minocycline topical suspension. In certain embodiments, the patient has from about 10 inflammatory lesions to about 100 inflammatory lesions on their face due to rosacea prior to starting treatment with minocycline topical suspension. In certain embodiments, the patient has from about 15 inflammatory lesions to about 70 inflammatory lesions on their face due to rosacea prior to starting treatment with minocycline topical suspension. In certain embodiments, the patient has from about 10 inflammatory lesions to about 50 inflammatory lesions on their face due to rosacea prior to starting treatment with minocycline topical suspension. In certain embodiments, the patient has at least 10, 15, 20, 25, 30, 35, 40, 45, 50, 55, 60, 65, or 70 inflammatory lesions on their face due to rosacea prior to starting treatment with minocycline topical suspension. In certain embodiments, the patient has at least 10 but no more than 30, 35, 40, 45, 50, 55, 60, 65, or 70 inflammatory lesions on their face due to rosacea prior to starting treatment with minocycline topical suspension.

In certain embodiments, the patient does not receive one or more of an anticoagulant, penicillin, a statin, or hormonal therapy while receiving minocycline topical suspension on a daily basis. In certain embodiments, the patient does not receive an anticoagulant, penicillin, a statin, or hormonal therapy while receiving minocycline topical suspension on a daily basis.

In certain embodiments, the rosacea is papulopustular rosacea. In certain embodiments, the rosacea is moderate-to-severe papulopustular rosacea.

### 4. Therapeutic Improvements & Other Characteristics

The method may be further characterized according to therapeutic benefits of administration of minocycline topical suspension to the patient. Exemplary therapeutic benefits that may be measured are described herein below.

### Reduction in Number of Inflammatory Lesions

The method may be further characterized according to the reduction in number of inflammatory lesions. For example, in certain embodiments, the method produces at least a 25% reduction in the number of inflammatory lesions on the patient's face. In certain embodiments, the method produces at least a 35% reduction in the number of inflammatory lesions on the patient's face. In certain embodiments, the method produces at least a 45% reduction in the number of inflammatory lesions on the patient's face. In certain embodiments, the method produces at least a 55% reduction in the number of inflammatory lesions on the patient's face. In certain embodiments, the method produces at least a 10, 15, 20, 25, 30, 35, 40, 45, 50, 55, 65, 70, 75, 80, 85, 90, or 95 percent reduction in the number of inflammatory lesions on the patient's face.

In certain embodiments, the method produces a reduction in inflammatory lesions in the amount of at least 10 inflammatory lesions on the patient's face. In certain embodiments, the method produces a reduction in inflammatory lesions in the amount of at least 15 inflammatory lesions on the patient's face. In certain embodiments, the method produces a reduction in inflammatory lesions in the amount of at least 20 inflammatory lesions on the patient's face. In certain embodiments, the method produces a reduction in inflammatory lesions in the amount of at least 10, 15, 20, 25, 30, 35, 40, 45, 50, or 55 inflammatory lesions on the patient's face.

### Reduction in Erythema Severity Score

The method may be further characterized according to the reduction in the Erythema Severity Score. For example, in certain embodiments, within a population of at least 20 patients the method produces a mean reduction in Erythema Severity Score of at least 0.5. In certain embodiments, the method produces a reduction in Erythema Severity Score of at least 1. In certain embodiments, the method produces a reduction in Erythema Severity Score of at least 2. In certain embodiments, the method achieves an Erythema Severity Score of absent or mild.

### Reduction in Telangiectasia Severity Score

The method may be further characterized according to the reduction in the Telangiectasia Severity Score. For example, in certain embodiments, within a population of at least 20 patients the method produces a mean reduction in Telangiectasia Severity Score of at least 0.5. In certain embodiments, the method produces a reduction in Telangiectasia Severity Score of at least 1. In certain embodiments, the method produces a reduction in Telangiectasia Severity Score of at least 2. In certain embodiments, the method achieves a Telangiectasia Severity Score of absent or mild.

### Time to Achieve, and Duration of Reductions

The method may be further characterized according to the time required from first receiving treatment to achieving the reductions described above (for example, reduction in number of inflammatory lesions, Erythema Severity Score, and/or Telangiectasia Severity Score) and/or the duration of the reduction after discontinuing daily treatment administration. For example, in certain embodiments, said reduction occurs within 4 weeks of first receiving minocycline topical suspension. In certain embodiments, said reduction occurs within 8 weeks of first receiving minocycline topical suspension. In certain embodiments, said reduction occurs within 12 weeks of first receiving minocycline topical suspension. In certain embodiments, said reduction occurs within 24 weeks of first receiving minocycline topical suspension. In certain embodiments, said reduction occurs within 2, 3, 4, 5, 6, 7, 8, 9, 10, 11, 12, 13, 14, 15, 16, 17, 18, 19, 20, 21, 22, 23, or 24 weeks of first receiving minocycline topical suspension.

In certain embodiments, said reduction lasts for a duration of at least 2 weeks after discontinuing daily administration of the minocycline topical suspension. In certain embodiments, said reduction lasts for a duration of at least 4 weeks after discontinuing daily administration of the minocycline topical suspension. In certain embodiments, said reduction lasts for a duration of at least 6 weeks after discontinuing daily administration of the minocycline topical suspension. In certain embodiments, said reduction lasts for a duration of at least 1, 2, 3, 4, 5, 6, 7, or 8 weeks after discontinuing daily administration of the minocycline topical suspension.

### Improvements in Investigators Global Assessment Score

The method may be further characterized according to the degree, time to achieve, and/or duration of improvement in the Investigators Global Assessment Score. For example, in certain embodiments, the method produces at least a one-grade improvement in Investigators Global Assessment score. In certain embodiments, the method produces at least a two-grade improvement in Investigators Global Assessment score. In certain embodiments, the method achieves an improvement in Investigators Global Assessment score resulting in an Investigators Global Assessment score of clear or almost clear.

In certain embodiments, said improvement in Investigators Global Assessment score occurs within 4 weeks of first receiving minocycline topical suspension. In certain embodiments, said improvement in Investigators Global Assessment score occurs within 8 weeks of first receiving minocycline topical suspension. In certain embodiments, said improvement in Investigators Global Assessment score occurs within 12 weeks of first receiving minocycline topical suspension. In certain embodiments, said improvement in Investigators Global Assessment score occurs within 24 weeks of first receiving minocycline topical suspension. In certain embodiments, said improvement in Investigators Global Assessment score occurs within 2, 3, 4, 5, 6, 7, 8, 9, 10, 11, 12, 13, 14, 15, 16, 17, 18, 19, 20, 21, 22, 23, or 24 weeks of first receiving minocycline topical suspension.

In certain embodiments, said improvement lasts for a duration of at least 2 weeks after discontinuing daily administration of the minocycline topical suspension. In certain embodiments, said improvement lasts for a duration of at least 4 weeks after discontinuing daily administration of the minocycline topical suspension. In certain embodiments, said improvement lasts for a duration of at least 6 weeks after discontinuing daily administration of the minocycline topical suspension. In certain embodiments, said improvement lasts for a duration of at least 1, 2, 3, 4, 5, 6, 7, or 8 weeks after discontinuing daily administration of the minocycline topical suspension.

### Limited Undesirable Side Effects

The method may be further characterized according to the limited undesirable side effects experienced by the patient due to the therapeutic methods. For example, in certain embodiments, any increase in Skin Discoloration Score is no greater than 1. In certain embodiments, any increase in Skin Discoloration Score is no greater than 2. In certain embodiments, any increase in Skin Pigmentation Score is no greater than 1. In certain embodiments, any increase in Skin Pigmentation Score is no greater than 2. In certain embodiments, any increase in Local Application Skin Reaction Score for erythema, dryness, erosion or edema is no greater than 1. In certain embodiments, any increase in Local Application Skin Reaction Score for erythema, dryness, erosion or edema is no greater than 2.

In certain embodiments, the patient does not experience any of headache, fatigue, dizziness, pruritus, liver injury, or hyperpigmentation due to minocycline topical suspension. In certain embodiments, the patient does not experience permanent skin discoloration due to minocycline topical suspension.

### Systemic Minocycline Exposure—Blood Plasma Pharmacokinetic Parameters

As described above, systemic exposure to minocycline increases the risk of adverse side effects. Accordingly, the methods may be further characterized according to minocycline blood plasma pharmacokinetic parameters. For example, in certain embodiments, the patient's minocycline blood plasma AUC_{0-24 hr} is less than 0.05 h*μg/mL. In certain embodiments, the patient's minocycline blood plasma AUC_{0-24 hr} is less than 0.03 h*μg/mL. In certain embodiments, the patient's minocycline blood plasma Cₘₐₓ is less than 1.9 ng/mL. In certain embodiments, the patient's minocycline blood plasma Cₘₐₓ is less than 1.6 ng/mL. In certain embodiments, any Tₘₐₓ for minocycline in the patient's blood plasma is in the range of 5 to 7 hours. In certain embodiments, any Tₘₐₓ for minocycline in the patient's blood plasma is about 6 hours. A Tₘₐₓ in the range of 4.5 to 7.5 hours is desirable for a topical minocycline suspension that is to be administered only once per day.

In certain embodiments, the concentration of minocycline in the patient's blood plasma is no greater than about 1.9 ng/mL three hours after administration of topical minocycline suspension. In certain embodiments, the concentration of minocycline in the patient's blood plasma is no greater than about 1.8 ng/mL three hours after administration of topical minocycline suspension. In certain embodiments, the concentration of minocycline in the patient's blood plasma is no greater than about 1.7 ng/mL three hours after administration of topical minocycline suspension. In certain embodiments, the concentration of minocycline in the patient's blood plasma is no greater than about 1.6 ng/mL three hours after administration of topical minocycline suspension. In certain embodiments, the concentration of minocycline in the patient's blood plasma is no greater than about 1.5 ng/mL three hours after administration of topical minocycline suspension.

In certain embodiments, the concentration of minocycline in the patient's blood plasma is no greater than about 1.9 ng/mL twelve hours after administration of topical minocycline suspension. In certain embodiments, the concentration of minocycline in the patient's blood plasma is no greater than about 1.8 ng/mL twelve hours after administration of topical minocycline suspension. In certain embodiments, the concentration of minocycline in the patient's blood plasma is no greater than about 1.7 ng/mL twelve hours after administration of topical minocycline suspension. In certain embodiments, the concentration of minocycline in the patient's blood plasma is no greater than about 1.6 ng/mL twelve hours after administration of topical minocycline suspension. In certain embodiments, the concentration of minocycline in the patient's blood plasma is no greater than about 1.5 ng/mL twelve hours after administration of topical minocycline suspension.

In certain embodiments, the concentration of minocycline in the patient's blood plasma is no greater than about 1.9 ng/mL, 1.8 ng/mL, 1.7 ng/mL, 1.6 ng/mL, or 1.5 ng/mL sixteen hours after administration of topical minocycline suspension. In certain embodiments, the concentration of minocycline in the patient's blood plasma is no greater than about 1.9 ng/mL, 1.8 ng/mL, 1.7 ng/mL, 1.6 ng/mL, or 1.5 ng/mL twenty-four hours after administration of topical minocycline suspension.

In certain embodiments, the concentration of minocycline in the patient's blood plasma is no greater than about 1.9 ng/mL, 1.8 ng/mL, 1.7 ng/mL, 1.6 ng/mL, or 1.5 ng/mL three hours after administration of topical minocycline suspension on the seventh consecutive day of administering topical minocycline suspension to the patient. In certain embodiments, the concentration of minocycline in the patient's blood plasma is no greater than about 1.9 ng/mL, 1.8 ng/mL, 1.7 ng/mL, 1.6 ng/mL, or 1.5 ng/mL twelve hours after administration of topical minocycline suspension on the seventh consecutive day of administering topical minocycline suspension to the patient.

In certain embodiments, the concentration of minocycline in the patient's blood plasma is no greater than about 1.9 ng/mL, 1.8 ng/mL, 1.7 ng/mL, 1.6 ng/mL, or 1.5 ng/mL three hours after administration of topical minocycline suspension on the fourteenth consecutive day of administering topical minocycline suspension to the patient. In certain embodiments, the concentration of minocycline in the patient's blood plasma is no greater than about 1.9 ng/mL, 1.8 ng/mL, 1.7 ng/mL, 1.6 ng/mL, or 1.5 ng/mL twelve hours after administration of topical minocycline suspension on the fourteenth consecutive day of administering topical minocycline suspension to the patient.

### Reduction in Transepidermal Water Loss

The method may be further characterized according to any reduction in transepidermal water loss from the patient's face due to the therapeutic methods. For example, in certain embodiments, the method produces a reduction in transepidermal water loss from the patient's face. In certain embodiments, the method produces at least a 10% reduction in transepidermal water loss from the patient's face. In certain embodiments, the method produces at least a 25% reduction in transepidermal water loss from the patient's face. In certain embodiments, the method produces at least a 1%, 2%, 3%, 4%, 5%, 6%, 7%, 8%, 9%, 10%, 15%, 20%, 25%, 30%, 35%, 40%, or 45% reduction in transepidermal water loss from the patient's face. In certain embodiments, said reduction in transepidermal water loss is measured at four weeks from the start of treatment using topical minocycline suspension. In certain embodiments, said reduction in transepidermal water loss is measured at 1, 2, 3, 4, 5, or 6 weeks from the start of treatment using topical minocycline suspension.

### Increase in Water Content of Patient's Skin

The method may be further characterized according to any increase in the water content of skin on the patient's face due to the therapeutic methods. For example, in certain embodiments, the method produces an increase in water content of skin on the patient's face. In certain embodiments, the method produces at least a 5% increase in water content of skin on the patient's face. In certain embodiments, the method produces at least a 15% increase in water content of skin on the patient's face. In certain embodiments, the method produces at least a 1%, 2%, 3%, 4%, 5%, 6%, 7%, 8%, 9%, 10%, 15%, 20%, 25%, 30%, 35%, 40%, or 45% increase in water content of skin on the patient's face. In certain embodiments, said increase in water content of skin on the patient's face is measured at four weeks from the start of treatment using topical minocycline suspension. In certain embodiments, said increase in water content of skin on the patient's face is measured at 1, 2, 3, 4, 5, or 6 weeks from the start of treatment using topical minocycline suspension.

### J. Methods of Treating Acne

Another aspect of the invention provides methods for treating acne. The methods comprise topically administering to a patient in need thereof a therapeutically effective amount of a minocycline topical suspension described herein. In certain embodiments, the acne is acne vulgaris. In certain embodiments, the acne features one or more of a whitehead, blackhead, papule, pustule, nodule, or cyst.

In certain embodiments, the invention provides a method of treating acne, wherein the method comprises topically administering to the face of a human patient in need thereof once per day at or near bedtime a dose of about 1 gram of minocycline topical suspension to treat the acne, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
   wherein particles of minocycline in the topical suspension have a D90 particle size in the range of from about 3 microns to about 6 microns, and the topical suspension comprises about 3% (w/w) minocycline.

In certain embodiments, a procedure described in a method described herein above for treating rosacea is used to treat acne. For example, in certain instances, the invention provides a method of treating acne, wherein the method comprises topically administering to the face of an adult human patient in need thereof once per day at or near bedtime a dose of about 1 gram of minocycline topical suspension to treat the acne, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil; wherein particles of minocycline in the topical suspension have a D90 particle size in the range of from about 3 microns to about 6 microns, and the topical suspension comprises about 3% (w/w) minocycline.

The method may be further characterized according to additional features, such as the weight percent minocycline in the topical suspension, the dose of minocycline suspension, and minocycline blood plasma pharmacokinetic parameters. For example, in certain embodiments, the topical suspension comprises 3% (w/w) minocycline. In certain embodiments, the dose is 1 g of minocycline topical suspension.

In certain embodiments, the patient's minocycline blood plasma AUC_{0-24 hr} is less than 0.1 h*μg/mL. In certain embodiments, the patient's minocycline blood plasma AUC_{0-24 hr} is less than 0.05 h*μg/mL. In certain embodiments, the patient's minocycline blood plasma AUC_{0-24 hr} is less than 0.02 h*μg/mL.

In certain embodiments, the patient's minocycline blood plasma Cₘₐₓ is less than 2.45 ng/mL. In certain embodiments, the patient's minocycline blood plasma Cₘₐₓ is less than 2.0 ng/mL. In certain embodiments, the patient's minocycline blood plasma Cₘₐₓ is less than 1.7 ng/mL. In certain embodiments, the patient's minocycline blood plasma Cₘₐₓ is less than 1.5 ng/mL.

In certain embodiments, any Tₘₐₓ for minocycline in the patient's blood plasma is in the range of 4.5 to 7.5 hours. The method may also be further characterized according to additional features described below.

Another aspect of the invention provides a method of treating acne, wherein the method comprises topically administering to the face of a patient in need thereof once per day a dose of from about 0.5 g to about 1.3 g of a minocycline topical suspension to treat the acne, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
   wherein particles of minocycline in the topical suspension have a D90 particle size less than 8 microns, and the topical suspension comprises from about 1% (w/w) to about 3% (w/w) minocycline.

### II. Compositions for Medical Use

Minocycline topical suspensions described herein may be used to treat a medical condition described herein. The use may be according to a method described herein. For example, one aspect of the invention provides a minocycline topical suspension for use in treating inflammatory lesions of rosacea by topical administration to the face of an adult human patient in need thereof once per day at or near bedtime a dose of about 1 gram of the minocycline topical suspension, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
   wherein particles of minocycline in the topical suspension have a D90 particle size in the range of from about 3 microns to about 6 microns, and the topical suspension comprises about 3% (w/w) minocycline.

Embodiments described herein in connection with the methods for treatment may be applied in connection with the minocycline topical suspensions for use.

### III. Preparation of a Medicament

Minocycline topical suspensions described herein may be used in the preparation of a medicament to treat a medical condition described herein. For example, one aspect of the invention provides for the use of a minocycline topical suspension described herein in the preparation of a medicament for treating inflammatory lesions of rosacea by topical administration to the face of an adult human patient in need thereof once per day at or near bedtime a dose of about 1 gram of the minocycline topical suspension, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
   wherein particles of minocycline in the topical suspension have a D90 particle size in the range of from about 3 microns to about 6 microns, and the topical suspension comprises about 3% (w/w) minocycline.

Embodiments described herein in connection with the methods for treatment may be applied in connection with the minocycline topical suspensions for use in the preparation of a medicament.

### IV. Medical Kits

Another aspect of the invention provides a medical kit comprising, for example, (i) a composition described herein, and (ii) instructions for treating rosacea according to methods described herein. Another aspect of the invention provides a medical kit comprising, for example, (i) a composition described herein, and (ii) instructions for reducing the number of inflammatory lesions according to methods described herein.

### EXAMPLES

The invention now being generally described, will be more readily understood by reference to the following examples, which are included merely for purposes of illustrating certain aspects and embodiments of the present invention, and are not intended to limit the invention.

### Example 1—Preparation of Minocycline Topical Suspension

Minocycline topical suspension was prepared according to the procedures described below.

General Procedure: Minocycline base crystalline Form II particles were added to mineral oil and the mixture was stirred for about 30 minutes. To the resulting mixture, polymeric hydrocarbon gelling agent was slowly added and the resulting suspension was stirred for 30 minutes to provide the final minocycline topical suspension. The polymeric hydrocarbon gelling agent used was the commercially available polymeric hydrocarbon gelling agent sold under the tradename VERSAGEL® M-750, which is a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol).

The minocycline topical suspensions in Tables 1 and 2 below were prepared based on the general procedure described above. The Vehicle in Table 3 was prepared by combining the indicated polymeric hydrocarbon gelling agent and mineral oil.

TABLE 1
Composition of 1% w/w Minocycline Topical Suspension

| Component | Amount | Function |
|---|---|---|
| Minocycline base | 1% w/w | active ingredient |
| Polymeric hydrocarbon gelling agent | 69.3% w/w | gelling agent |
| commercially available under the | | |
| tradename VERSAGEL ® M-750 | | |
| Mineral oil | 29.7% w/w | wetting agent |

TABLE 2
Composition of 3% w/w Minocycline Topical Suspension

| Component | Amount | Function |
|---|---|---|
| Minocycline base | 3% w/w | active ingredient |
| Polymeric hydrocarbon gelling agent | 67.9% w/w | gelling agent |
| commercially available under the | | |
| tradename VERSAGEL ® M-750 | | |
| Mineral oil | 29.1% w/w | wetting agent |

TABLE 3
Composition of Vehicle

| Component | Amount |
|---|---|
| Polymeric hydrocarbon gelling agent | 70.0% w/w |
| commercially available under the | |
| tradename VERSAGEL ® M-750 | 30.0% w/w |
| Mineral oil | |

### Example 2—Stability Study of 3% w/w Minocycline Topical Suspension

Stability of 3% w/w minocycline topical suspension was analyzed. Experimental procedures and results are described below.

### Part I—Experimental Procedures

Minocycline 3% topical gel was packed in aluminum tubes and stored at ICH stability storage conditions (accelerated: 40° C.±2° C./75% RH±5%; intermediate: 30° C.±2° C./65% RH±5%, and long-term: 25° C.±2° C./60% RH±5%). Stability of the topical gel was determined using the following tests: description, package integrity, minocycline identification, assay and related substances, content uniformity, particle size distribution, viscosity, and microbial limits. Minocycline identification, assay, related substances, and content uniformity was determined by using a validated HPLC method.

### Part II—Results

Summary of the key stability results are described in Table 4 below; results of all other tests were found to be compliant for 36 months at long-term storage conditions.

TABLE 4
Results of Stability Study of 3% w/w Minocycline Topical Suspension

| | | Storage | Duration (Months) | | | | | | | |
|---|---|---|---|---|---|---|---|---|---|---|
| Test | Initial | Conditions | 1 | 3 | 6 | 9 | 12 | 18 | 24 | 36 |
| Minocycline: | 100.4 | Long-term | — | 102.3 | 105.0 | 102.8 | 103.6 | 99.3 | 101.1 | 100.2 |
| Assay (% w/w | | Intermediate | — | — | 100.9 | 99.9 | 97.4 | — | — | — |
| of Label Claim) | | Accelerated | 103.3 | 100.9 | 103.0 | — | — | — | — | — |
| 4-epi Minocycline | 0.69 | Long-term | — | 0.84 | 0.86 | 0.85 | 0.89 | 1.30 | 1.44 | 0.90 |
| (% w/w) | | Intermediate | — | — | 1.03 | 1.20 | 1.40 | — | — | — |
| | | Accelerated | 0.90 | 1.1 | 1.61 | — | — | — | — | — |
| Total Impurities | 0.18 | Long-term | — | 0.60 | 0.60 | 0.63 | 0.79 | 1.03 | 0.92 | 0.53 |
| (excluding 4-epi- | | Intermediate | — | — | 0.69 | 0.57 | 0.89 | — | — | — |
| minocycline) (% | | Accelerated | 0.69 | 0.66 | 0.94 | — | — | — | — | — |
| w/w) | | | | | | | | | | |

Storage Conditions: Long-term (25° C. ± 2° C./60% RH ± 5%);
Accelerated (40° C. ± 2° C./75% RH ± 5%);
Intermediate (30° C. ± 2° C./65% RH ± 5%).
—: Not part of the stability testing timepoint.

### Example 3—Stability Study of 1% w/w Minocycline Topical Suspension

Stability of 1% w/w minocycline topical suspension was analyzed. Experimental procedures and results are described below.

### Part I—Experimental Procedures

Minocycline 1% topical gel was packed in aluminum tubes and stored at 25° C.±2° C./60% RH±5%. Minocycline identification, assay, and related substances were determined by using a validated HPLC method.

### Part II—Results

Summary of the key stability results are described in Table 5 below. The drug product is stable for up to 36 months at room temperature.

TABLE 5
Results of Stability Study of 1% w/w Minocycline Topical Suspension

| | | Duration (Months) | | | | | | |
|---|---|---|---|---|---|---|---|---|
| Test | Inital | 3 | 6 | 9 | 12 | 18 | 24 | 36 |
| Assay (minocycline, | 99.4 | 103.5 | 106.2 | 99.9 | 101.2 | 99.2 | 101.3 | 99.8 |
| % w/w) | | | | | | | | |
| 4-epiminocycline | 0.7 | 0.96 | 1.01 | 0.90 | 1.22 | 1.49 | 1.34 | 1.08 |
| (% w/w) | | | | | | | | |

### Example 4—Treatment of Papulopustular Rosacea in Human Subjects Using Minocycline Topical Suspension

The ability of minocycline topical suspension to treat a human subject suffering from papulopustular rosacea was evaluated according to the clinical study described below, in which minocycline topical suspension is administered to the face of the patient once daily just prior to bedtime, and then the patient was evaluated for improvement. Experimental procedures and results are described below.

### A. Study Summary

In order to evaluate the efficacy and safety of two strengths of minocycline topical suspension (Test Article) versus vehicle administered once daily for twelve weeks in subjects with a diagnosis of papulopustular rosacea, the following procedures were conducted.

This was a multicenter, randomized, double-blind, parallel group, vehicle-controlled study that evaluated the safety and efficacy of 1% and 3% topical minocycline gel in patients with papulopustular rosacea. The abbreviation HY01 used herein refers to minocycline. Two concentration levels of minocycline in a topical gel applied once-daily were evaluated, compared to vehicle, over a 12-week treatment period in patients with moderate-to-severe papulopustular rosacea. Study subjects returned for the follow-up visit on Week 16 to participate in the assessments of treatment efficacy persistence.

During the baseline visit, the Investigator or designee recorded subject's demographics, medical history, reviewed concomitant medication, identified any prohibited therapies the subject may receive, performed a physical examination, collected vital signs and performed a urine pregnancy test. Eligible patients were randomized in a ratio of 1:1:1 to receive either HY01 Topical Gel 1%, HY01 Topical Gel 3%, or vehicle (contains 0% w/w minocycline) treatment and were trained on the application of the study drug and subject's diary completion. The Investigator or approved designee performed inflammatory lesion count. The Investigator performed the Investigator's Global Assessment (IGA), assessed the severity of erythema and telangiectasia and completed Local Application Skin Reaction Score and Questionnaire to reflect the baseline skin condition.

Subjects were advised on use of specific cleanser and general precautions related to the study. Randomization and study drug kit number assignment were performed using an Interactive Web Response System (IWRS). The study drug was applied once daily at bedtime over a 12-week treatment period. Subjects were instructed to wash their entire face with recommended cleanser and pat dry with a soft towel before applying the study drug. A thin layer of study treatment (approximately 1 gram (i.e., five fingertip units)) was gently massaged into the entire face at bedtime. Fingertip unit was defined as an amount of study drug applied from the distal skin-crease to the tip of the index finger (see FIG. 7). Subjects were instructed to avoid contact with the mouth, eyes and other mucous membranes, and that their hands should be washed before and after application. Subjects were advised to refrain from washing or wiping their faces for at least four hours post application, and to use the study cleanser to wash off the study drug in the morning. The subject was given a sunscreen to be used for protection from excessive outdoor sun exposure during daylight hours, if required.

Subjects returned for post-baseline visit assessments at Days 7, 28, 42, 63 and 84, returning used tubes of the study drug at each visit, which were accounted for along with diary cards, which were reviewed by the site personnel for completeness. The tubes were weighed and the weight recorded. An Investigator performed inflammatory lesion count, Investigator's Global Assessment (IGA), Investigator's Global Assessment including Erythema (IGAe), assessed the severity of erythema and telangiectasia, and recorded local application site reactions. Study subjects were asked to complete a Self-Assessment Questionnaire, and to record tolerability of treatment and concerns of discoloration. Urine pregnancy testing was repeated at Day 42 and 84 visits.

At the end of the treatment period, the End of Treatment (EOT) visit was performed, which included physical examination and urine pregnancy test.

Subjects were asked to return to the study center again 4 weeks after the EOT for follow-up efficacy assessments, which included inflammatory lesion count, Investigator's Global Assessment (IGA), completion of the Erythema Severity Scale, Telangiectasia Severity Scale and a Self-Assessment Questionnaire.

Additional consent information for photography was provided with the study ICF, which allowed the subject to agree to participate in the study but decline to provide photographs. For those subjects who consented to photography, standardized photographs were taken at selected sites using a professional quality digital camera. Such photographs were used for illustrative purposes only.

A subset of approximately 30 subjects was enrolled in the pharmacokinetic evaluation. These subjects received a pre-dose blood draw at baseline prior to any treatment. They then completed the study treatment period. Following the completion of the End of Treatment visit (day 84) assessments, these subjects received an additional dose of the study product at the clinic; the study product was the same tube that these subjects had been using for their treatment phase. Subjects who did not complete the treatment phase were not included in the pharmacokinetic analysis. Blood draws were taken prior to the additional dose application at the clinic and then at 1, 2, 4, 6, 20, and 24 hours following the day 84 additional dosing. Blood draws at the 20 and 24 hour post-dose time points were collected at the day 85 visit. Study subjects and phlebotomy staff were instructed to not touch the treatment area following minocycline gel application and to wash hands thoroughly prior to all blood draws. The blood draw was to be completed in the antecubital fossa.

The planned overall sample size for this clinical trial was approximately 249 adult male and female patients with moderate to severe papulopustular rosacea, who were randomized at a 1:1:1 ratio (HY01 1%:HY01 3%:vehicle) at about 23 clinical research centers in the United States. Approximately 30 subjects at approximately 5 of the clinical sites were enrolled in the pharmacokinetic analysis. Approximately 6 subjects on Vehicle, 12 subjects on HY01 Gel, 1%, and 12 subjects on HY01 Gel, 3% at approximately 5 sites were included in the pharmacokinetic analysis.

### B. Investigational Product (IP)

Test Article was supplied as 30 grams of yellow gel in an aluminum tube with a nasal tip and a plastic cap closure, labeled for a blinded study. Subjects randomized to the Vehicle control arm received the same tubes containing all components at the concentrations used in the Test Article except for the active component. The compositions of the gels are described in the Tables below. Particles of minocycline in the HY01 Gels had a D90 particle size within the range of from 3 microns to 4 microns, and a D50 particle size within the range of from 1 to 2 microns.

TABLE 6
Composition of HY01 Gel, 1% (Minocycline Gel, 1%)

| Component | Amount | Function |
|---|---|---|
| Minocycline base | 1% w/w | active ingredient |
| Polymeric hydrocarbon gelling agent | 69.3% w/w | gelling agent |
| commercially available under the | | |
| tradename VERSAGEL ® M-750 | | |
| Mineral oil | 29.7% w/w | wetting agent |

TABLE 7
Composition of HY01 Gel, 3% (Minocycline Gel, 3%)

| Component | Amount | Function |
|---|---|---|
| Minocycline base | 3% w/w | active ingredient |
| Polymeric hydrocarbon gelling agent | 67.9% w/w | gelling agent |
| commercially available under the | | |
| tradename VERSAGEL ® M-750 | | |
| Mineral oil | 29.1% w/w | wetting agent |

TABLE 8
Composition of Vehicle (Vehicle, Vehicle Gel, or Placebo)

| Component | Amount | Function |
|---|---|---|
| Polymeric hydrocarbon gelling agent | 70.0% w/w | gelling agent |
| commercially available under the | | |
| tradename VERSAGEL ® M-750 | | |
| Mineral oil | 30.0% w/w | wetting agent |

The minocycline base was minocycline base in crystalline Form II. The polymeric hydrocarbon gelling agent commercially available under the tradename VERSAGEL® M-750 was a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol).

### C. Study Objectives and Endpoints

The primary objective of the study was to evaluate the efficacy of once-daily application of 1% and 3% minocycline topical gel, as assessed by the change in inflammatory lesion count from baseline over 12-week treatment period in patients with moderate-to-severe papulopustular rosacea. Secondary objectives of the study were to evaluate the:
Efficacy of once-daily application of 1% and 3% minocycline topical gel, in improving the Investigator's Global Assessment (IGA) scores from baseline over 12-week treatment period in patients with moderate-to-severe papulopustular rosacea.
Efficacy of once-daily application of 1% and 3% minocycline topical gel, in improving the Investigator's Global Assessment including Erythema (IGAe) scores from baseline over 12-week treatment period in patients with moderate-to-severe papulopustular rosacea.
Safety and tolerability of once-daily application of minocycline topical gel as assessed by the occurrence, frequency and severity of adverse events, local application skin reactions, skin discoloration scores and change in vital signs and physical examination over the 12-week treatment period in patients with moderate-to-severe papulopustular rosacea.
Persistence of therapeutic effect of once-daily application of 1% and 3% minocycline topical gel, versus vehicle, over the 12-week treatment period, as defined by the inflammatory lesion count and Investigator's Global Assessment scores comparison between the end of treatment (Week 12) visit and follow-up visit (Week 16).
Pharmacokinetic properties of the investigational drug product.

The primary endpoint of the study was absolute change in the inflammatory (papules and pustules) lesion count from the baseline to Week 12. Secondary endpoints of the study were:
Success on Investigator Global Assessment (IGA), defined as two or more-point reduction or improvement to “clear” or “almost clear” at Week 12.
Success on Investigator Global Assessment including Erythema (IGAe), defined as two or more-point reduction or improvement to “clear” or “almost clear” at Week 12.
Percent change in the inflammatory (papules and pustules) lesion count from the baseline to Week 12.

Exploratory endpoints of the study were:
Erythema Severity Scale score at Week 12 compared to the baseline.
Telangiectasia Severity Scale score at Week 12 compared to the baseline.
Subject satisfaction with treatment tolerability and concerns of discoloration as determined by the Self-Assessment Questionnaire score at each study visit beginning with Week 1.
Absolute change in the in the inflammatory (papules and pustules) lesion count from the baseline to the follow-up visit (Week 16).
Two or more-point reduction of Investigator's Global Assessment (IGA) score or improvement to “clear” or “almost clear” at the follow-up visit (Week 16) compared to the baseline.
Absolute change in the inflammatory lesion count from baseline to week 12 of the treatment groups versus vehicle and between treatment groups.

Pharmacokinetic endpoints of the study were to determine minocycline systemic pharmacokinetic properties, such as area under the curve (AUC), maximum concentration (Cmax), time to maximum concentration (Tmax), and half-life (T½), after 12 weeks of once-daily topical application of minocycline topical gel.

Safety endpoints of the study were the frequency and severity of adverse events by treatment group, physical examination, and vital signs; and change in local application skin reaction scores and skin discoloration scores at Week 12, compared to baseline.

### D. Study Eligibility Criteria

Subjects who satisfied all of the following inclusion criteria and had none of the following exclusion criteria were allowed to enroll in the study. Inclusion criteria for the study were:
Subjects had provided written informed consent to participate in the study.
Male or non-pregnant female aged ≥18 years with a clinical diagnosis of moderate to severe facial rosacea, defined as the presence of:
   At least twelve and not more than forty inflammatory facial lesions (i.e., papules/pustules), and
   Subjects with a grade 3 or 4 on the 5-point Investigators Global Assessment (IGA) scale, and
   Persistent facial erythema (scored as at least mild on Erythema Severity Scale), and
   Facial telangiectasia (scored as at least mild on Telangiectasia Severity Scale).
Subject was willing to minimize external factors that might trigger rosacea flare-ups as recommended per protocol and patient instructional guide (e.g., spicy foods, thermally hot foods and drinks, hot environments, prolonged sun exposure, strong winds and alcoholic beverages).
Non-nursing, female subjects of child bearing potential, who were using an acceptable form of birth control: total abstinence; oral (birth control pills); intravaginal (e.g. NuvaRing®), implantable (e.g. Norplant®), injectable (e.g. Depo-Provera®) or transdermal (e.g. Ortho Evra®) contraception; intrauterine device (IUD); double-barrier (diaphragm or condom with spermicidal gel or foam); for two months prior to study enrollment or a vasectomized partner. All female subjects of child bearing potential must have undergone an in-office urine pregnancy test, with a negative result, prior to being randomized to receive study drug. In addition, women of childbearing potential must have agreed to a have urine pregnancy test at Day 42 and at the end of the study (Day 84). Females not of childbearing potential due to menopause must have been postmenopausal for at least one year. Male subjects must have been willing to not attempt to conceive a child during the participation in the study.
Subjects who used the same brand of soap, make-up, hair products, or shaving lotion/foam/cream/gel for a period of at least four weeks prior to the Baseline Visit and agreed not to change these product brand/types during the study, with the exception of using the Ponds Cold Cream cleanser provided by the sponsor.
Male subjects who were willing to shave, if applicable, at approximately the same time every day.
Subjects who were willing to refrain from sunbathing, using sun tanning booths/beds, or excessive exposure to the sun for the duration of the study.

The exclusion criteria for the study were:
1. Presence of any skin condition on the face that would have interfered with the diagnosis or assessment of rosacea as determined by the Investigator. Excessive facial hair (e.g. beards, sideburns, moustaches, etc.) that would interfere with diagnosis or assessment of rosacea. Patients also must have agreed not to grow excess facial hair during the study (i.e. they needed to be free of excess facial hair for follow-up visits).
2. History of hypersensitivity or allergy to minocycline, any other tetracycline, or any other component of the formulation, or known reactions to cleansers, including Ponds Cold Cream, and sunscreen.
3. Subjects who were using, or planning use of, concomitant treatments within 30 days of the Baseline Visit (e.g., facial or chemical peels, dermal fillers, acne surgery, intralesional steroids, spironolactone, debridement, cryotherapy, dermabrasion, IPL, X-ray, laser therapy or UV therapy).
4. Use within 6 months prior to the Baseline Visit of oral retinoids (e.g. isotretinoin, acitretin) or therapeutic vitamin A supplements of greater than 10,000 units/day. (Multivitamins were allowed)
5. Subjects who were using estrogens or progestin agents (e.g., Gynogen, Valergen, Depo-Testadiol, Depogen, birth control pills), for less than 2 months prior to the Baseline Visit. (Subjects using estrogens for 2 months or more were not excluded unless the subject expected to change dose, drug, or discontinue estrogen use during the study).
6. Use within 2 month prior to the Baseline Visit of 1) topical retinoids to the face, 2) systemic antibiotics known to have an impact on the severity of facial rosacea (e.g., containing tetracycline and its derivatives, erythromycin and its derivatives, sulfamethoxazole, metronidazole or trimethoprim), or 3) systemic corticosteroids.
7. Use within 2 months prior to the Baseline Visit of 1) topical corticosteroids, 2) topical antibiotics or 3) topical medications for rosacea (e.g., metronidazole, azelaic acid, erythromycin, ivermectin, sulfur based topical products).
8. Subjects with rhinophyma, dense telangiectasia, or plaque-like facial edema, more than 5 nodules or sinus tracts.
9. Ocular rosacea (e.g., conjunctivitis, blepharitis, or keratitis) of sufficient severity to require topical or systemic antibiotics.
10. Subjects with underlying diseases or other dermatological conditions, such as; atopic dermatitis, perioral dermatitis, or seborrheic dermatitis, which required the use of interfering topical or systemic therapy or may have interfered with the rosacea diagnosis.
11. Subjects who were using an investigational drug or participating in an investigational study within 30 days of the Baseline Visit. Use of an investigational drug and/or participation in another investigational study was prohibited during this study.
12. Subjects who were currently abusing alcohol or drugs, or who had a history of chronic alcohol or drug abuse within the past year.
13. Medical history of immunodeficiency or other significant ongoing medical condition or disease as determined by the investigator.

### Withdrawal and Early Discontinuation

A subject could voluntarily withdraw from the study at any time and for any reason without prejudice to his or her future medical care. The Investigator, Sponsor, or Medical Monitor could also withdraw a subject at any time if it was medically necessary or in the interest of subject's safety. Additional possible reasons for premature discontinuation of study drug included adverse events and major non-compliance with study procedures, as described below.

The withdrawal of a subject from study drug by the Investigator was discussed with the Medical Monitor before the subject stopped study drug, whenever possible.

A subject was discontinued from this study if any of the following criteria were met:
Withdrawal of consent by the subject was received.
In the opinion of the Investigator, Medical Monitor or Sponsor, it was not in the subject's best interests to continue in the study.
Occurrence of an Adverse Event (AE) or Serious Adverse Event (SAE), which, in the opinion of the Investigator, warranted discontinuation of the subject from the study.
Pregnancy as informed by the subject or as determined by a positive urine pregnancy test, which were conducted at the baseline, Week 6, and Week 12 EOT/ET visits for females of childbearing potential.
Significant non-compliance with study procedures that would have interfered with the study results or increased the subject's risks in the study, as determined by the Investigator.
If subject was deemed to be a treatment failure, which was defined as worsening of condition and requiring alternate or supplemental therapy for the treatment of rosacea during the study.

Subjects who discontinued early from the study due to lack of treatment effect after completing at least eight weeks of treatment were included in the mITT and PP population as treatment failures and the change in inflammatory lesion count from the baseline visit to the last completed visit prior to discontinuation due to lack of efficacy were carried forward in the primary endpoint analysis. Subjects discontinued early for other reasons were excluded from the PP population.

If a subject discontinued prematurely, the Investigator performed the Early Termination (ET) visit, capturing the reason for discontinuation and performing assessments of the EOT visit.

If a subject did not return for a scheduled visit, every effort was made to contact the subject and document the End of Study visit assessments. The Investigator was required to document the primary reason for discontinuation of a study subject in the source document and on the appropriate electronic case report form (eCRF).

Randomized subjects who discontinued due to an adverse event had all events documented and followed to satisfactory resolution.

### Contraception and Pregnancy Avoidance Measures

Female subjects must have been post-menopausal, surgically sterile or using highly effective birth control methods with a negative urine pregnancy exam at the Baseline Visit. Acceptable forms of contraception included: total abstinence; oral (birth control pills); intravaginal: (e.g. NuvaRing®), implantable (e.g. Norplant®), injectable (e.g. Depo-Provera®) or transdermal (e.g. Ortho Evra®) contraception; intrauterine device (IUD); double-barrier (diaphragm or condom with spermicidal gel or foam); for two months prior to study enrollment or a vasectomized partner (6 months status post vasectomy). Women using oral contraceptives were also required to utilize a secondary form of contraception during the study.

Male subjects must have been willing to not attempt to conceive a child during the participation in the study by utilization of barrier contraception.

If a subject, or female partner of a male subject, became pregnant during the participation in the study, the Investigator was instructed to immediately discontinue the patient from the study and contact the Medical Monitor and the Sponsor. Diligent efforts were made to determine the outcome for all pregnancy exposures in the clinical trial. Information on the status of the mother and the child were forwarded to the Sponsor's Safety Team using the Pregnancy Data Collection Form. Generally, follow-up was supposed to occur within 6 to 8 weeks following the estimated delivery date. Any premature termination of the pregnancy was reported.

### Prior, Concomitant, and Prohibited Therapy

Current medications and any medications taken within the 30 days prior to the start of the study were recorded as prior/concomitant medications (using their generic name, if known) with the corresponding indication, start and stop dates. The medications to be recorded included prescription and all over-the-counter (OTC) medications and all dietary supplements. All medications taken on a regular basis, including aspirin and acetaminophen, should have been recorded prior to commencing the use of the investigational product. Any medications started during the study (including “as needed” medications) were recorded in the concomitant medication list as soon as the Investigational Site became aware of the medication being added.

If an Investigator became aware of a subject having taken a prohibited medication or therapy, as described below, they were to report the incident to the Medical Monitor within 24 hours, and the Medical Monitor and/or Sponsor was to provide written approval of the subject's continuation or discontinuation from the study.

Therapies prohibited within 6 months prior to the Baseline Visit and throughout the study were oral retinoids (e.g. isotretinoin, acitretin) or therapeutic vitamin A supplements of greater than 10,000 units/day. Multivitamins were allowed.

Treatment with estrogens or progestational agents (e.g., Gynogen, Valergen, Depo-Testadiol, Depogen, birth control pills) for less than 2 months prior to the Baseline Visit was prohibited. Subjects using estrogens for 2 months or more were not excluded unless the subject expected to change dose, drug, or discontinue estrogen use during the study.

Therapies prohibited within 2 months of the Baseline Visit and throughout the study were:
Topical retinoids to the face.
Systemic and topical antibiotics known to have an impact on the severity of facial rosacea (e.g., containing tetracycline and its derivatives, erythromycin and its derivatives, sulfamethoxazole, or trimethoprim).
Systemic and topical corticosteroids (including eye drops, asthma inhalers, and nasal sprays, if not on stable dosing).
Topical medications for rosacea (e.g., metronidazole, azelaic acid, ivermectin, sulfur based topical products).

Therapies prohibited within 30 days of the Baseline Visit and throughout the study were concomitant treatment with facial or chemical peels, dermal fillers, acne surgery, intralesional steroids, spironolactone, debridement, cryotherapy, dermabrasion, X-ray, laser therapy or UV therapy.

Therapies and activities prohibited throughout the study were sunbathing or using a sunlamp; use of occlusive dressings or wrappings in treatment areas; and change in moisturizers, new brands of make-up, creams, lotions or powders other than the assigned treatment to the treatment area.

### E. Study Procedures

The following procedures were completed during the study. Whenever possible, subjects were to be assessed by the same Investigator or site personnel.

### Treatment Assignment (Randomization)

Randomization was performed using an Interactive Web Response System (IWRS) at the time the subject met all eligibility criteria and was ready for enrollment. Subjects were randomized in a 1:1:1 ratio to receive HY01 Gel, 1%, HY01 Gel, 3%, or Vehicle. Pharmacokinetic subjects were continued on initial study treatment and were randomized 2:2:1 to HY01 Gel, 1%, or HY01 Gel, 3%, or Vehicle. Approximately 6 subjects on Vehicle, 12 subjects on HY01 Gel, 1%, and 12 subjects on HY01 Gel, 3% were included in the pharmacokinetic analysis.

The randomization scheme was generated and kept strictly confidential throughout the conduct of the study and as not available to the Sponsor, Investigator, or study staff, or to clinical staff who could have had an impact on the outcome of the study. The randomization scheme included investigative site and lesion count strata (<25 or ≥25). Pharmacokinetic investigational sites were stratified by lesion count only. The subset of subjects who consented for one additional dosing on day 84 for pharmacokinetic assessment remained on the treatment assignment, per the initial randomization for the treatment phase.

### Blinding

This was a double-blind study, thus Sponsor, Investigator and clinical study site staff, study monitors, and subjects were blinded to the randomization scheme. The packaging of the study drug products was similar in appearance to make difference in treatment less obvious to the subjects. The blinding scheme for each study drug product was supplied in a sealed envelope with each individual set; it was available to FDA or other regulatory agency investigators at the time of site inspection to allow for verification of the treatment identity of each subject.

The blinding code was not to be broken except in emergency situations for which the identification of the study treatment of a subject may have been required by the Investigator to complete a Serious Adverse Event (SAE) report. In such situations, the Medical Monitor or the Investigator was to use the IWRS system in order to unblind the treatment for the individual subject. Unblinded information was to be held by designated individual(s), and the date and reason for breaking the blind were to be recorded.

The Medical Monitor was to be contacted by telephone prior to unblinding but no later than 24 hours after unblinding. As the study was blinded, the Investigator was to promptly document and explain to the Sponsor any premature unblinding (e.g., accidental unblinding, unblinding due to a SAE) of the investigational study product(s).

### Study Drug Dispensation and Application

Study subjects were dispensed a tube of the study drug at the Baseline, Day 28, Day 42, and Day 63 visits). Subjects received the randomized treatment along with a diary card, written instructions and precautions. Subjects also received Pond's Cold Cream Cleanser and moisturizer with sunscreen (or similar product) at these visits.

Each subject was to be instructed by the Investigator or designated staff on proper application technique. The Investigator was to advise treatment of the entire face using approximately five fingertip units during the application training session at the Baseline Visit, and was to demonstrate the appropriate amount required for one application, which was equal to approximately 1 gram (or five fingertip units) of the study drug product. The sponsor-supplied moisturizer with sunscreen was used for demonstration of the application during the training session.

The following application instructions were to be provided to the subject:
1. At bed-time, before applying the study product, wash entire face with warm water (NOT HOT) using a mild cleanser or soap. Pat face dry with soft towel.
2. Squeeze one application on index fingertip equivalent to approximately one inch in length. You will need 5 fingertip applications in total.
3. Apply the study drug to 5 separate sections on the face (one squeeze or fingertip per section): the left and right temples, left and right cheeks, and chin.
4. Gently massage study drug into the skin on the entire face. Avoid contact with eyes, mouth and the inside of the nose. After application, wash hands thoroughly with mild soap. Leave the study drug on the face overnight
5. Wash the study drug off in the morning, or no less than 6 hours after application, using the provided Ponds Cold Cream cleanser.

Subjects were to be instructed to:
Protect their faces with the provided moisturizer with sunscreen when spending time in the sun.
Wash their hands prior to and following the investigational drug application procedures.
Avoid exposure to extreme cold weather conditions, when possible, to avoid rosacea flare-ups.
Limit intake of spicy food and alcohol throughout the duration of the study.

The subject was dispensed two tubes of study drug at the Baseline visit. The tubes were weighed, and the weight of each tube was recorded (to the nearest 0.1 gram) prior to dispensing to the subject. The subject was required to return both tubes of study drug at each visit and the study site was to weigh and record the weight (to the nearest 0.1 gram) of each tube at each visit. Additional tubes of study drug were dispensed to the subject at Day 28, Day 42, and Day 63 to ensure the subject had an adequate supply of study drug at all times. The site was to use the IWRS system for study drug dispensation.

### Treatment Compliance

Treatment compliance was assessed by the Investigator or designated staff based on diary card completion at each study visit following the baseline visit. Compliant subjects were defined as those who had completed 75% treatment applications and had not missed more than 5 consecutive days of dosing.

The subject was given a Diary Card at each study visit of the treatment period to record applications of the study drug. Subjects recorded every application during the treatment period and were to bring the Diary Card to each of the post-baseline study visits for compliance review. The Investigator was to verify that the subject complied with the application requirements and was to file the completed Diary Card in the study files at each study visit until the EOT/ET visit. Any missed application notations were clarified with the subject and documented.

### Documentation of Screen Failures

Investigators were required to account for all subjects who sign informed consent and were to maintain an Enrollment Log capturing subjects screened and indicating who was enrolled or excluded and the reason why. If the subject was found not to be eligible prior to enrollment, the reason(s) for ineligibility was to be documented by the Investigator. Subject Numbers assigned to subjects who fail screening were not be re-used.

### Clinical Evaluations

The following clinical evaluations were conducted during the course of the study.

### Subject Demographics

Basic demographic information, including date of birth, Fitzpatrick skin type, sex, ethnicity, and race were recorded at the Baseline Visit.

### Medical History

Medical history was collected at the Baseline Visit. Relevant medical history, including past history of rosacea were documented. Presence of immunodeficiency, or other clinically significant ongoing medical condition or disease would disqualify subject from participation.

### Concomitant Medication Recording

All medications (both prescription and nonprescription, and including vitamins, herbals, topicals, inhaled, and intranasal) taken within 30 days prior to the start of the Study Drug and through the final study visit were recorded on the appropriate eCRF (using their generic name, if known) with the corresponding indication, start and stop dates. At each study visit, subjects were asked whether they had started or discontinued any medication since their previous study visit. This included single use or PRN (as needed) medication use. Previous treatment of rosacea was to be recorded irrespective of the duration it was given. Corresponding condition was to be captured in the subject's Medical History.

### Vital Signs

Vital signs were collected at each study visit. Vital signs included body temperature, heart rate and blood pressure (systolic and diastolic). Blood pressure and pulse rate were measured after the subject had been sitting restfully for at least 5 minutes.

Any abnormal characteristics were evaluated by the Investigator based on their significance. Abnormal vital signs were considered AEs if they required therapeutic medical intervention, and/or if the Investigator considered them to be AEs, based on his/her clinical judgement.

### Physical Examination

Physical examination, including height, weight, and evaluation of organs and systems (General Appearance, Heart/Cardiovascular, Lungs, Gastrointestinal, Ears/Nose/Throat, Extremities, and Skin) were to be assessed at the Baseline and EOT/ET visits. A subject with abnormalities other than the presence of rosacea lesions, which in the opinion of the Investigator, may pose a risk to the safety of the subject, or may interfere with the assessment of safety or efficacy in the study, were to be excluded.

### Recording of Inflammatory Lesions Count

The area of lesion count assessment was defined as the face from the jaw line to the hairline. The lesion counts were performed at each study visit (Baseline, Weeks 1, 4, 6, 9, 12 and 16 (Follow-Up Visit)). The lesion counts were performed as follows:
Subjects were instructed to wash their face and remove all makeup at least 15 minutes prior to assessment. Male subjects must have shaven before coming for the study visit.
Subjects were seated at arm's length (or slightly closer) from the assessor performing the lesion counts and at the same height as the assessor. Subjects were not to be seated in direct sunlight.
The room was to have balanced and consistent artificial light source or natural light from a North-facing window.
The Investigators or approved designee participating in this study were instructed to perform all lesion counts. It was essential that the same assessor performed all lesion counts for a given subject for the duration of that subject's participation in the study.
Lesions on each area of the face were to be considered separately. Lesions were counted just below the hairline and no lower than the mandibular line (jaw line). The face was systematically scanned from left to right and lesions were counted and recorded. All lesions counted were palpated. If a lesion was not palpable it was considered non-active (emerging or resolving). The inflammatory lesion count included the total count of papules and pustules, which was defined as follows:
   PAPULE—A type of inflammatory lesion; a small erythematous, palpable lesion, usually solid <0.5 cm in diameter.
   PUSTULE—A type of inflammatory lesion; a small palpable lesion with an erythematous base less than 0.5 cm in size and containing pus or yellow-white liquid.
   NODULE—A type of inflammatory lesion; a large palpable erythematous papule or plaque that was greater than 0.5 cm in diameter.

Papules were counted separately from pustules (inflammatory lesions). All lesion types were counted separately for each quadrant on the face. Counts of nodules were to be reported separately and not included in the inflammatory lesion counts.

### Investigator's Global Assessment (IGA)

An Investigator's Global Assessment (IGA) was evaluated at each study visit beginning from the Baseline Visit, as described in the Table below. This scale was not to be a reflection of treatment response, but was to describe the condition at each visit. Therefore, no reference was to be made to baseline in the evaluation.

TABLE 9
Investigator’s Global Assessment (IGA) Scale

| Score | Grade | Definition |
|---|---|---|
| 0 | Clear | No inflammatory lesions present |
| 1 | Almost Clear | Very few small papules/pustules |
| 2 | Mild | Several small papules/pustules |
| 3 | Moderate | Several small or large papules/pustules, and up to 2 nodules |
| 4 | Severe | Numerous small and/or large papules/pustules, up to several nodules |

Investigator's Global Assessment including Erythema (IGAe)

An Investigator's Global Assessment including Erythema (IGAe) was evaluated at each study visit beginning from the Baseline Visit, as described in the Table below. This scale was not to be a reflection of treatment response, but was to describe the condition at each visit. Therefore, no reference was to be made to baseline in the evaluation.

TABLE 10
Investigator’s Global Assessment including Erythema (IGAe) Scale

| Score | Grade | Definition |
|---|---|---|
| 0 | Clear | No inflammatory lesions present; at most, mild erythema |
| 1 | Almost Clear | Very mild erythema present. Very few small papules/pustules |
| 2 | Mild | Mild erythema. Several small papules/pustules |
| 3 | Moderate | Moderate erythema. Several small or large papules/pustules, and up to 2 nodules. |
| 4 | Severe | Severe erythema. Numerous small and/or large papules/pustules, up to several nodules. |

### Erythema Severity Score

The Investigator was to grade the severity of non-transient facial erythema from 0 to 3, e.g. 0=none, 1=Mild, 2=Moderate, 3=Severe at each study visit beginning from the Baseline Visit, as described in the Table below. Although inflammation (papules, pustules, plaques) or dry appearance may obscure the level of erythema, underlying redness was to be evaluated disregarding this effect. Inflammation or dry appearance was allowed to be noted, but perilesional erythema was not to be included in this assessment. Transient erythema (flushing) was also not be included.

TABLE 11
Erythema Severity Scale

| Score | Grade | Definition |
|---|---|---|
| 0 | Absent | No perceptible erythema. |
| 1 | Mild | Slight erythema with either restricted central |
| | | involvement or generalized whole face. |
| 2 | Moderate | Pronounced erythema with either restricted central |
| | | involvement or generalized whole face. |
| 3 | Severe | Severe erythema or red-purple hue with either restricted |
| | | central involvement or generalized whole face. |

### Telangiectasia Severity Score

Facial telangiectasia were graded at each study visit beginning from the Baseline Visit by the Investigator from 0 to 3 using the Telangiectasia Severity Scale, as described in the Table below.

TABLE 12
Telangiectasia Severity Scale

| Score | Grade | Definition |
|---|---|---|
| 0 | Absent | No telangiectasia |
| 1 | Mild | Slight telangiectasia characterized by a few |
| | | fine, small red vessels 0.2 mm or less in diameter; |
| | | telangiectasia covers less than 10% of face |
| 2 | Moderate | Pronounced telangiectasia characterized |
| | | by several fine vessels and/or by a few |
| | | large vessels 0.2 mm or greater in diameter; |
| | | telangiectasia covers between 10-30% of face |
| 3 | Severe | Severe telangiectasia characterized by |
| | | many fine vessels and/or large vessels; |
| | | telangiectasia covers more than 30% of face |

### Safety and Tolerability Assessments

The following evaluations of safety and tolerability were conducted during the course of the study.

### Assessment of Adverse Events

Beginning with the Week 1 visit and through the EOT/ET and follow-up visits, the Investigator and study personnel were to review each subject's clinical evaluation findings and query the subject directly regarding AEs (see below). Subjects were to be followed for AEs until the final required protocol visit or until all drug-related toxicities and SAEs had resolved (or are considered chronic/stable), whichever was later.

### Local Application Skin Reaction Score

Local application skin reaction scores for erythema, dryness, erosion, and edema. Investigators will score the erythema, dryness, erosion, and edema thought to be secondary to the medication on a scale of 0-3, where 0=absent, 1=mild (slight, barely perceptible), 2=moderate (distinct presence), 3=severe (marked, intense). Subjects were asked to assess the tolerability of the medication and record their answer on the questionnaire presented as a Table below.

### Skin Discoloration Score

Skin discoloration (staining) was assessed by the Investigator at each post-baseline visit of the treatment period using a five-point ordinal scale, as described in the Table below. The purpose of the skin discoloration scale was to identify discoloration secondary to the minocycline active product, which has a yellow tint. Post-inflammatory pigment change, actinic damage, and ethnic pigmentation were all to be ignored for the purpose of this rating.

TABLE 14
Skin Discoloration Scale (Yellowing or Discoloration Presumed to be
Secondary to the Investigational Drug Product)

| Score | Grade | Definition |
|---|---|---|
| 0 | None | No discoloration |
| 1 | Minimal | Minimal discoloration of the skin, barely perceptible |
| 2 | Slight | Slight, but definite discoloration of the skin |
| 3 | Moderate | Moderate discoloration of the skin, clearly perceptible at a glance |
| 4 | Pronounced | Pronounced discoloration of the skin over much of the application site |
| 5 | Very | Very pronounced, strong discoloration of the skin over the whole application site |
| | Pronounced | |

### Subject's Self-Assessment Questionnaire

Subjects were asked to complete a Self-Assessment Questionnaire at each of the post-baseline visits, capturing subject satisfaction with tolerability and staining concerns since the last visit on a questionnaire such as the one presented as a Table below.

### Subject's Investigation Site Visits

The Screening Visit included the following procedures and assessments:
Informed Consent review and documentation, including additional consent information for applicable subjects for pharmacokinetics and photography.
Collection of Demographics and Medical History
Assessment of Vital Signs
Completion of Clinical Scales (Investigator's Global Assessment, Investigator's Global Assessment including Erythema, Erythema Severity Scale, Telangiectasia Severity Scale)
Recording inflammatory lesion count (papules, pustules and nodules)
Concomitant and Prohibited Therapies review and documentation

If no medication wash-out period was required, Screening and Baseline visits can occur on the same day.

The Baseline Visit (Day 0) included the following procedures and assessments:
Informed Consent review and documentation
Urine pregnancy test for female subjects of childbearing potential
Collection of Demographics and Medical History
Concomitant and Prohibited Therapies review and documentation
Assessment of Vital Signs
Physical examination
Photographs of subject face (select subjects only)
Completion of Clinical Scales (Investigator's Global Assessment, Investigator's Global Assessment including Erythema, Erythema Severity Scale, Telangiectasia Severity Scale)
Recording inflammatory lesion count (papules, pustules and nodules)
Confirmation of Eligibility
Randomization
Training on Study Drug Application and Diary Card Completion
Recording weight of two Study Drug tubes and Dispensation to the subject
Dispensation of cleanser and moisturizer with sunscreen
Baseline blood draw for pharmacokinetic participants.
Subjects were asked to apply study drug at bed-time.

Visits #2-5 (Day 7 [Week 1], Day 28 [Week 4], Day 42 [Week 6], and Day 63 [Week 9]) included the following procedures and assessments:
Adverse Events assessment
Concomitant and Prohibited Therapies review
Collection of Vital Signs
Diary Card completion review and collection
Recording inflammatory lesion count (papules, pustules and nodules)
Recording local application site reactions
Completion of Clinical Scales (Investigator's Global Assessment, Investigator's Global Assessment including Erythema, Erythema Severity Scale, Telangiectasia Severity Scale)
Skin Discoloration Score (observer reported)
Photographs of subject face (Visits 3 and 6, select subjects only)
Completion of Subject Satisfaction Questionnaire and Local Reaction Questionnaire
Collect Study Drug tubes from subject, weigh, and record weight to the nearest 0.1 grams
Re-dispense Study Drug tubes to subject and instruct on proper application technique (Re-supply of Study Drug dispensed to subject on Day 28, Day 42, and Day 63)
Day 42 [Week 6] will also include the urine pregnancy testing for females of childbearing potential.

Visit #6a (End of Treatment/Early Termination (EOT/ET) visit (Day 84 [Week 12])) included, in addition to activities described above for Visits #2-5, the following procedures and assessments:
Physical examination
Urine pregnancy test
For prematurely discontinuing subjects—record reason for discontinuation, if applicable.

For subjects enrolled in the pharmacokinetic assessment, immediately following the completion of activities above, the following activities were performed:
Pre-dose blood draw
In-clinic dosing of the study product
Post-dose blood draws at 1, 2, 4, 6, 20, and 24 hours following dose application. (The 20 and 24 hour blood draw were performed at Visit 6b.)
Reminded the subject to return to the site on Day 85 for the 20 and 24 hour post-dose blood draws.

Visit #6b (Day 85 [Week 12]) was conducted only for subjects enrolled in the pharmacokinetic assessment. Site staff members were to ensure timing of the Day 85 visit was within protocol specified PK collection time points. The following activities were performed:
Adverse Event Assessment
Concomitant and Prohibited Therapies review
Collection of Vital Signs
Post-dose blood draw at 20 and 24 hours following dose application.

Visit #7 (Follow-up Visit (Day 112 [Week 16])) included the following procedures and assessments:
Recording an inflammatory lesion count (papules, pustules and nodules)
Completion of Clinical Scales (Investigator's Global Assessment, Investigator's Global Assessment including Erythema, Erythema Severity Scale, Telangiectasia Severity Scale)
Adverse Events assessment.

Subjects were encouraged to report any complications or adverse effects during their participation. Investigators were allowed to evaluate the subject at an unscheduled visit, if subject's condition was considered to be worsening.

### F. Safety Data Collection, Recording, and Reporting

Investigators were to monitor each subject for clinical evidence of adverse events on a routine basis throughout the study. The Investigators were to assess and record any Adverse Event (AE) in detail including the date of onset, description, severity, time course, duration and outcome, relationship of the adverse event to study drug, an alternate etiology for events not considered “related” or “probably related” to study drug, final diagnosis, if known, and any action(s) taken. For AEs to be considered intermittent, the events must have been of similar nature and severity and each intermittent AE was reported separately. AEs and Serious Adverse Events (SAEs), whether in response to a query, observed by site personnel, or reported spontaneously by the subject were recorded, monitored and followed-up until the resolution (or until the Investigator deemed the event to be stable/chronic).

An Adverse Event (AE) was classified as any untoward medical occurrence in a patient or clinical investigation subject administered a pharmaceutical product, including those which may not have a causal relationship with this treatment. An AE could therefore be any unfavorable and unintended sign (including an abnormal laboratory finding), symptom, or disease temporally associated with the use of a medicinal (investigational) product, whether or not the event was considered causally related to the use of the investigational product.

Such an event could result from use of the drug as stipulated in the protocol or labeling, as well as from accidental or intentional overdose, drug abuse, or drug withdrawal. Any worsening of a pre-existing condition or illness was considered an AE. For example, if a new rosacea sign or symptom, or worsening of a rosacea sign or symptom, was believed by the investigator to be related to the study drug and not the disease, then it was recorded as an AE. Clinically significant abnormalities were to be followed to resolution (i.e. become stable, return to normal, return to baseline, or become explainable). Laboratory abnormalities and changes in vital signs were considered to be AEs only if they necessitated therapeutic medical intervention, and/or if the Investigator considered them to be AEs.

A Serious Adverse Event (SAE) was an AE meeting any of the following criteria:
Death of Subject—An event that resulted in the death of a subject.
Life-Threatening—An event that, in the opinion of the Investigator, would have resulted in immediate fatality if medical intervention had not been taken. This did not include an event that would have been fatal if it had occurred in a more severe form.
Hospitalization—An event that resulted in an admission to the hospital for any length of time. This did not include an emergency room visit or admission to an out-patient facility.
Prolongation of Hospitalization—An event that occurred while the study subject was hospitalized that prolonged the subject's hospital stay.
Congenital Anomaly/Birth Defect—An anomaly detected at or after birth, or any anomaly that resulted in fetal loss.
Persistent or Significant Disability/Incapacity—An event that resulted in a condition that substantially interfered with the activities of daily living of a study subject. Disability was not intended to include experiences of relatively minor medical significance, such as headache, nausea, vomiting, diarrhea, influenza, and accidental trauma (e.g., sprained ankle).
Other Important Medical Event—An important medical event that may not have been immediately life-threatening or resulted in death or hospitalization, but based on medical judgment may have jeopardized the subject and may have required medical or surgical intervention to prevent any of the outcomes listed above (i.e., death of subject, life-threatening, hospitalization, prolongation of hospitalization, congenital anomaly, or persistent or significant disability/incapacity). Examples of such events include allergic bronchospasm requiring intensive treatment in an emergency room or at home, blood dyscrasias or convulsions that did not result in inpatient hospitalization, or the development of drug dependency or drug abuse.

Any SAE, i.e. AE meeting any of the criteria above, was to be reported to the Sponsor's Safety department and Pharmacovigilance, using SAE report form, within 24 hours of occurrence or notification to the study site.

The severity of each AE and SAE was to be rated by the Investigator using the following classifications:
Mild—The event was transient and easily tolerated by the subject.
Moderate—The event caused the subject discomfort and interrupted the subject's usual activities.
Severe—The event caused considerable interference with the subject's usual activities and may have been incapacitating or life-threatening.

The causality of each AE and SAE (i.e. the relationship of the AE or SAE to the use of the investigational product) was to be assessed by the Investigator using the following classifications:
Related—The event occurred within a reasonable time after drug administration or drug concentration and body fluids demonstrated that the study drug was present: the event could not be reasonably explained by known characteristics including concomitant therapies; the adverse event abated after discontinuing the study drug.
Probably Related—The event has a strong temporal relationship to study drug or recurs on re-challenge and another etiology was unlikely or significantly less likely.
Possibly Related—The event has a strong temporal relationship to the study drug and an alternative etiology was equally or less likely compared to the potential relationship to study drug.
Probably Not Related—The event has little or no temporal relationship to the study drug and/or a more likely alternative etiology exists.
Not Related—The event was due to an underlying or concurrent illness or effect of another drug and is not related to the study drug (e.g., has no temporal relationship to study drug or has a much more likely alternative etiology).

If an Investigator's opinion of possibly, probably not, or not related to study drug was given, an alternate etiology must have been provided by the Investigator for the AE.

The adverse event collection period started with the Baseline visit through the follow-up visit (week 16). Any AE/SAE prior to the Baseline visit was considered past medical history (PMH). Adverse events that occurred during the treatment period (starting from the day of the first application of Study Drug and up to the final protocol required visit) were considered Treatment Emergent AEs (TEAEs). SAE(s) that were observed or spontaneously reported during the subject's participation in the trial were captured and monitored until the Investigator deemed the event to be chronic or not clinically significant or the subject to be stable.

Treatment assignment for an individual subject was to be unblinded only in an emergency by the Investigator, when knowledge of the treatment assignment was urgently needed for the clinical management or welfare of the subject. Treatment was to be provided in accordance with the medical condition and with regard to the information provided in the Investigator's Brochure. Reasons to unblind a subject were to be clearly documented in subject notes, and the monitoring site visit report.

### G. Data Analysis

Descriptive statistical methods were used to summarize the data from this study, with confidence intervals calculated for the primary and secondary efficacy endpoints. Unless stated otherwise, the term “descriptive statistics” refers to number of subjects (n), mean, median, standard deviation (SD), minimum, and maximum for continuous data, and frequencies and proportions for categorical data. The term “treatment group” refers to randomized treatment assignment: active-test and active-reference. All data collected during the study was included in data listings. Unless otherwise noted, the data was sorted first by treatment assignment, subject number, and then by date within each subject number.

Unless specified otherwise, all statistical testing was two-sided and was performed using a significance (alpha) level of 0.05.

For the purpose of using site as covariate, sites contributing with a low number of randomized patients were pooled with a comparable site based on geographical location or other characteristics. The approach for pooling was defined before unblinding.

### Subject Population/Data Sets to be Evaluated

The subject populations were defined as follows:
Safety Population—The safety population included all randomized subjects who received study drug.
Modified Intent-to-Treat (mITT) Population—A modified intent-to-treat population included all subjects who are randomized, applied at least one dose of assigned product, and returned for at least one post-baseline evaluation visit.
Per Protocol (PP) Population—All randomized subjects who met all inclusion/exclusion criteria, were compliant with the assigned study treatment, returned to the study site for the primary endpoint visit within the specified window (±4 days) OR discontinued from the study as a treatment failure, and did not have any protocol violations. Compliant subjects are defined as those who have completed 75% of treatment applications and have not missed more than 5 consecutive days of dosing.
Pharmacokinetic (PK) Population—All randomized subjects who received study treatment and at least one blood sample draw.

Subjects who discontinued early from the study due to lack of treatment effect after completing at least eight weeks of treatment were included in the mITT and PP population as treatment failures, and the change in inflammatory lesion count from the baseline visit to the last completed visit prior to discontinuation due to lack of efficacy was carried forward in the primary endpoint analysis. Subjects discontinued early for other reasons were excluded from the PP population, but included in the mITT population.

The results in the mITT population were considered definitive for superiority of each active treatment to vehicle with those in the PP population considered supportive. Safety analyses were performed using the Safety population.

### Efficacy Assessments

The primary efficacy endpoint was defined as the change from baseline to Week 12 of the Treatment Period with respect to the lesion count. The absolute change in inflammatory lesion count from baseline to Week 12 was analyzed using an analysis of covariance (ANCOVA) with factors for treatment and investigational site and baseline lesion count (<25 or ≥25) as covariate. Change from baseline for each treatment group was estimated using least square means. Estimates were presented with 95% confidence intervals and tests were 2 sided with significance level of 0.05.

Secondary efficacy endpoints were analyzed as follows:
For the IGA and IGAe, score success was defined as an improvement in score to “clear” or “almost clear” or at least 2-grade improvement from baseline. The IGA and IGAe scores were analyzed using logistic regression with factors for treatment and investigational site.
Percent change in the inflammatory (papules and pustules) lesion count from the baseline to Week 12 were analyzed using ANCOVA.
Change from baseline for each treatment and change from baseline for 1% respective 3% compared to placebo were estimated using least square means. Estimates were presented with 95% confidence intervals. Tests were two-sided with significance level 0.05.

Hypothesis testing was conducted for each of the two doses studied (1% and 3%), and each was compared against vehicle. The estimates of treatment effect and p-values resulting from these hypothesis tests were used, in addition to a review of safety data, to select the most appropriate dose for further development. For testing of hypotheses, the P-value for test of no difference between 1% respective 3% dose group compared to vehicle was reported. 1% and 3% dose groups were compared and reported. No correction for multiplicity was applied.

All efficacy endpoints were reported by treatment and visit. For the primary and secondary analysis missing values are imputed using LOCF. No correction for multiplicity was made.

### Safety Assessments

The reporting of safety data was descriptive, and included all subjects who received at least one dose of investigational product. The variables for safety endpoints were AEs, Local Application Site Reactions and vital signs measurements. AEs were summarized based on the frequency of AEs and their severity for all treated subjects. All AEs were coded using Medical Dictionary for Regulatory Activities (MedDRA) and summarized by treatment group. Data were summarized using preferred term and primary system organ class.

If a subject experienced multiple events that mapped to a single preferred term, the greatest severity and strongest Investigator assessment of relation to study drug were assigned to the preferred term for the appropriate summaries. If an event had a missing severity or relationship, it was classified as having the highest severity and/or strongest relationship to study drug.

Summaries of treatment-emergent AEs included any AEs reported beginning with the first dose of study drug on Day 1. The occurrence of treatment-emergent adverse events was summarized by treatment group using preferred terms, system organ classifications, and severity. Separate summaries of treatment-emergent serious adverse events, treatment-emergent adverse events related to study drug, and events leading to the discontinuation of study drug were generated. All adverse events reported were listed for individual subjects showing both verbatim and preferred terms.

Local skin reaction scores for erythema, dryness, burning/stinging, erosion, edema, pain, itching and bleeding were summarized by the severity and treatment group.

Concomitant medications were coded using the World Health Organization (WHO) dictionary. These data were summarized by treatment group. Previous and concomitant medications were presented in a data listing.

### Exploratory Endpoints

Erythema Severity Scale score at Week 12 compared to the baseline was analyzed using an analysis of covariance (ANCOVA) with factors for treatment and investigational site and baseline Erythema Severity Scale score.

Telangiectasia Severity Scale score at Week 12 compared to the baseline was analyzed using an analysis of covariance (ANCOVA) with factors for treatment and investigational site and baseline Telangiectasia Severity Scale score.

Subject satisfaction with treatment tolerability and concerns of discoloration across all treatment arms was determined by the Self-Assessment Questionnaire score at each study visit beginning with Week 1 and tabulated by treatment visit.

Absolute change in the inflammatory (papules and pustules) lesion count from the baseline to the follow-up visit (Week 16) were analyzed using an analysis of covariance (ANCOVA) with factors for treatment and investigational site and baseline lesion count (<25 or ≥25) as covariate.

Two or more-point reduction of Investigator's Global Assessment (IGA) or IGAe scores or improvement to “clear” or “almost clear” at the follow-up visit (Week 16) was compared to the baseline. The IGA and IGAe scores were analyzed using logistic regression with factors for treatment and investigational site.

The efficacy of once-daily application of 1% and 3% minocycline topical gel, between treatment groups and versus vehicle, was assessed by the change in inflammatory lesion count from baseline over the 12-week treatment period in patients with moderate-to-severe papulopustular rosacea.

Pharmacokinetic variables (plasma concentrations and pharmacokinetic parameters) were summarized by treatment using appropriate descriptive statistics. Mean and individual concentration versus time curves were plotted for each treatment in both linear and semi-log linear scales.

### Part II—Results

Results of the clinical study are described below.

### Demographics

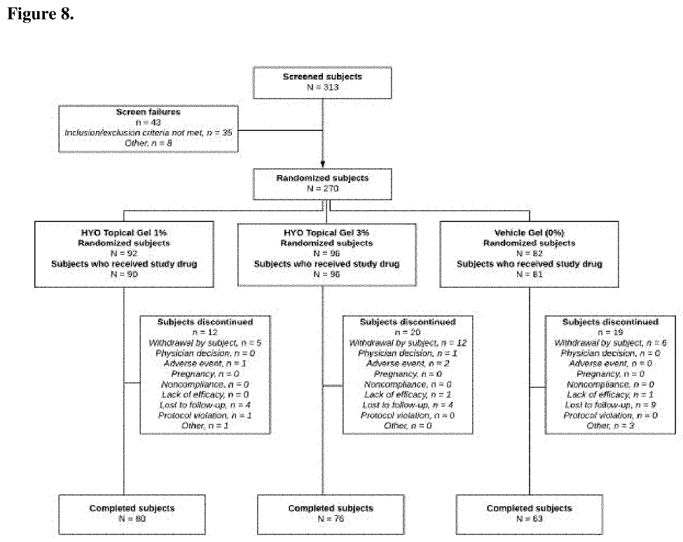

Baseline demographics are displayed in the Table below. The baseline demographics were similar in each study arm. There were 270 subjects enrolled in the intent to treat population, 92 randomized to 1% minocycline, 96 randomized to 3% minocycline, and 82 randomized to vehicle. The mean age was 51 years old, with 70% female and 30% male. At baseline, the subjects had a mean of 0.2 nodules, 21 papules, and 3.7 pustules. The average total inflammatory lesion count (papules and pustules) was 24.6, 25.1, and 24.3 in the minocycline 1%, 3%, and vehicle arms. There were 270 subjects randomized into the study, and 219 subjects completed the study. The disposition of subjects is shown in FIG. 8.

TABLE 16
Subject Demographics and Baseline Characteristics of the ITT Population

| | | | |
|---|---|---|---|
| HY01 1% | HY01 3% | Vehicle | Overall |
| (N = 92) | (N = 96) | (N = 82) | (N = 270) |

Age (years)

| | | | | |
|---|---|---|---|---|
| N | 92 | 96 | 82 | 270 |
| Mean (SD) | 51.5 (12.48) | 50.0 (13.49) | 52.0 (14.35) | 51.1 (13.41) |
| Median | 53.0 | 48.5 | 54.0 | 51.0 |
| Minimum, Maximum | 20, 84 | 19, 81 | 22, 86 | 19, 86 |

Sex, n (%)

| | | | | |
|---|---|---|---|---|
| Female | 62 (67.4) | 71 (74.0) | 56 (68.3) | 189 (70.0) |
| Male | 30 (32.6) | 25 (26.0) | 26 (31.7) | 81 (30.0) |

Race, n ( %)

| | | | | |
|---|---|---|---|---|
| White | 88 (95.7) | 94 (97.9) | 80 (97.6) | 262 (97.0) |
| Black or African American | 2 (2.2) | 0 | 0 | 2 (0.7) |
| Asian | 2 (2.2) | 1 (1.0) | 2 (2.4) | 5 (1.9) |
| American Indian or Alaska Native | 0 | 0 | 0 | 0 |
| Native Hawaiian or Other Pacific Islander | 0 | 1 (1.0) | 0 | 1 (0.4) |
| Other | 0 | 0 | 0 | 0 |

Ethnicity, n (%)

| | | | | |
|---|---|---|---|---|
| Hispanic or Latino | 28 (30.4) | 27 (28.1) | 19 (23.2) | 74 (27.4) |
| Non-Hispanic or Latino | 64 (69.6) | 69 (71.9) | 63 (76.8) | 196 (72.6) |

Fitzpatrick Skin Type, n (%)

| | | | | |
|---|---|---|---|---|
| I | 13 (14.1) | 10 (10.4) | 16 (19.5) | 39 (14.4) |
| II | 36 (39.1) | 30 (31.3) | 21 (25.6) | 87 (32.2) |
| III | 30 (32.6) | 45 (46.9) | 35 (42.7) | 110 (40.7) |
| IV | 10 (10.9) | 9 (9.4) | 10 (12.2) | 29 (10.7) |
| V | 2 (2.2) | 2 (2.1) | 0 | 4 (1.5) |
| VI | 1 (1.1) | 0 | 0 | 1 (0.4) |

Baseline Nodules

| | | | | |
|---|---|---|---|---|
| Mean (SD) | 0.2 (0.56) | 0.2 (0.64) | 0.2 (0.55) | 0.2 (0.76) |
| Minimum, Maximum | 0, 4 | 0, 3 | 0, 3 | 0, 8 |

Baseline Papules

| | | | | |
|---|---|---|---|---|
| N | 92 | 96 | 82 | 270 |
| Mean (SD) | 20.8 (7.89) | 21.5 (7.93) | 20.8 (7.94) | 21.0 (7.91) |
| Minimum, Maximum | 0, 39 | 6, 40 | 9, 40 | 0, 40 |

Baseline Pustules

| | | | | |
|---|---|---|---|---|
| N | 92 | 96 | 82 | 270 |
| Mean (SD) | 3.8 (6.18) | 3.7 (5.12) | 3.5 (4.12) | 3.7 (5.23) |
| Minimum, Maximum | 0, 38 | 0, 33 | 0, 18 | 0, 38 |

Baseline Total Inflammatory (Papules and Pustules) Lesion Count

| | | | | |
|---|---|---|---|---|
| N | 92 | 96 | 82 | 270 |
| Mean (SD) | 24.6 (7.41) | 25.1 (7.90) | 24.3 (8.20) | 24.7 (7.83) |
| Minimum, Maximum | 12, 40 | 12, 40 | 12, 40 | 12, 40 |

Baseline IGA Score

| | | | | |
|---|---|---|---|---|
| 0 =Clear | 0 | 0 | 0 | 0 |
| 1 = Almost Clear | 0 | 0 | 0 | 0 |
| 2 = Mild | 7 (7.6) | 3 (3.1) | 6 (7.3) | 16 (5.9) |
| 3 = Moderate | 75 (81.5) | 80 (83.3) | 69 (84.1) | 224 (83.0) |
| 4 = Severe | 10 (10.9) | 13 (13.5) | 7 (8.5) | 30 (11.1) |

Baseline IGAe Score

| | | | | |
|---|---|---|---|---|
| 0 = Clear | 0 | 0 | 0 | 0 |
| 1 = Almost Clear | 0 | 0 | 0 | 0 |
| 2 = Mild | 0 | 0 | 0 | 0 |
| 3 = Moderate | 85 (92.4) | 86 (89.6) | 74 (90.2) | 245 (90.7) |
| 4 = Severe | 7 (7.6) | 10 (10.4) | 8 (9.8) | 25 (9.3) |

### Inflammatory Lesions

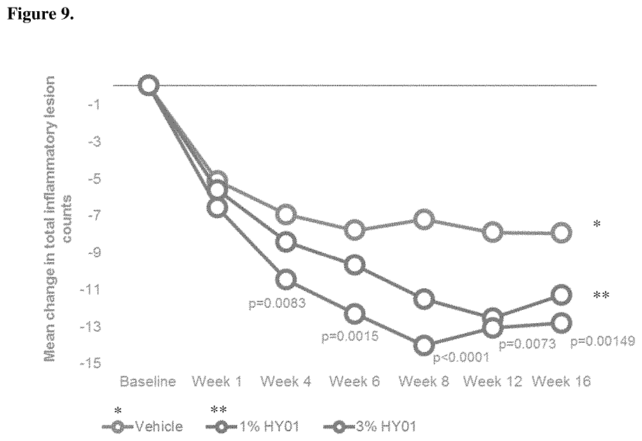

The primary outcome measure of the study was decrease in absolute inflammatory lesion count at week 12 compared to baseline. In the 1% and 3% minocycline arms the mean decrease was 12.64 and 13.09 respectively. The vehicle arm had a mean decrease of 7.92 inflammatory lesions. The decrease in the 1% and 3% arms were significantly greater than the vehicle arm (p=0.0147 and p=0.0073, respectively). FIG. 9 shows the decrease in inflammatory lesion count for each arm at each time point. The 3% minocycline group achieved a significantly greater reduction in inflammatory lesions compared to vehicle at week 4, which persisted throughout the remainder of the study. The 1% minocycline group achieved significance at week 9 and maintained significance compared to vehicle throughout the treatment period. Subjects were followed for 4 weeks following cessation of treatment and a significant reduction in inflammatory lesion was maintained in the 3% minocycline arm, but not in the 1% minocycline arm, compared to vehicle.

### Investigators Global Assessment (IGA)

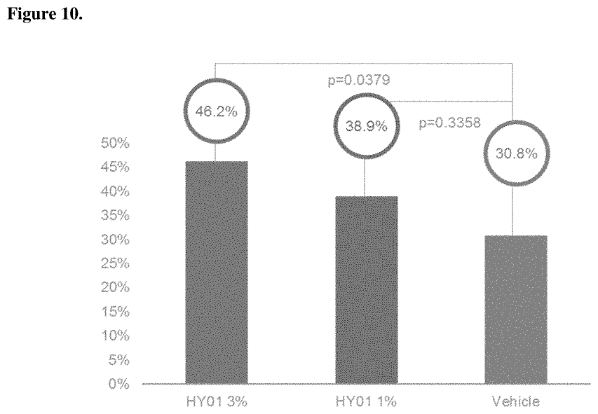

Success on the investigators global assessment was achieved at week 12 in 38.9%, 46.2%, and 30.8% of subjects in the 1% minocycline, 3% minocycline, and vehicles arms, respectively. Compared to vehicle, 3% minocycline had a significantly greater percentage of subjects achieving success on the IGA (p=0.0379). The proportion of subjects achieving success in each study arm is shown in FIG. 10. The 3% minocycline group achieved a significantly greater proportion of subjects achieving success compared to vehicle at day 63 (p=0.0306). This significance persisted through the duration of the treatment period and persisted to the last visit, 4 weeks following cessation of treatment (p=0.0302).

Investigators Global Assessment including Erythema (IGAe)

When using the IGAe, there was no statistically significant differences in the percentage of subjects achieving a success between the treatment groups at week 12. At week 12, 35.6%, 38.7%, and 28.2% of subjects achieved success in the 1% minocycline, 3% minocycline, and vehicle arms, respectively (p=0.3997 and p=0.1521 vs vehicle).

### Erythema Severity Score

Erythema was measured on a 4-point scale from none to severe at each treatment visit. The change from baseline in erythema severity at week 12 was −0.4, −0.6, and −0.4 for the 1% minocycline, 3% minocycline, and vehicle groups, respectively. The 3% minocycline group had a significantly greater reduction in erythema severity score compared to vehicle (p=0.0390).

### Subject Satisfaction and Safety

When asked “Do you like the product?” 72.3%, 69.6%, and 49.2% of subjects responded “Yes” at day 84 in the 1% minocycline, 3% minocycline, and vehicle arms, respectively. Treatment related treatment-emergent adverse events (Treatment related TEAEs) are reported in the Table below. There were 3 Treatment related TEAE's in the 1% minocycline arm, 5 in the 3% minocycline arm, and 1 in the vehicle arm. These adverse events were typically mild and transient. In the 1% minocycline and 3% minocycline groups, 87% and 79% of subjects completed the study. In the vehicle group, 76% of subjects completed the study. Overall the minocycline gel appeared to be well tolerated and safe.

TABLE 17
Treatment Related Treatment-Emergent Adverse Events

| Adverse Event | Minocycline 1% | Minocycline 3% | Vehicle |
|---|---|---|---|
| Nausea | 0 | 1 (1.0%) | 0 |
| Application site dermatitis | 1 (1.1%) | 0 | 0 |
| Application site erythema | 0 | 1 (1.0%) | 0 |
| Application site pruritus | 1 (1.1%) | 1 (1.0%) | 0 |
| Hypersensitivity | 0 | 1 (1.0%) | 0 |
| Headache | 0 | 1 (1.0%) | 0 |
| Rosacea | 0 | 1 (1.0%) | 1 (1.2%) |
| Urticaria | 0 | 1 (1.0%) | 0 |
| Total number | 3 (3.3%) | 5 (5.2%) | 1 (1.2%) |
| of subjects | | | |
| having at least | | | |
| one event (%) | | | |

### Pharmacokinetic Evaluation

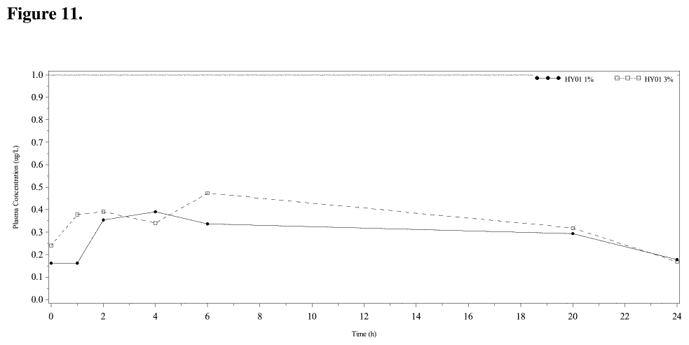

At week 12, a subset of 30 subjects were included in the PK portion of the study. The subjects had blood draws at baseline, then 1, 2, 4, 6, 20, and 24 hours following the dose. The lower limit of quantification (LLOQ) was 0.999 ng/mL. There were 13 subjects in the HY01 1% arm who completed the pharmacokinetic evaluation. Four subjects in this group had plasma concentrations above the LLOQ. These four subjects had a mean maximum concentration (Cₘₐₓ) of 1.411 ng/mL (SD 0.170) with a mean time of maximal concentration (Tₘₐₓ) of 8.467 hours and a mean area under the curve (AUC_{0-24 hr}) of 0.023 h*μg/mL. In the HY01 3% arm, 11 subjects completed the pharmacokinetic evaluation. Of the 11 subjects in the pharmacokinetic evaluation, 3 had plasma minocycline levels above the LLOQ. Among these 3 subjects, the mean Cₘₐₓ was 1.420 ng/mL (SD 0.178), Tₘₐₓ of 6 hours, and a mean AUC_{0-24 hr} of 0.021 h*μg/mL. The plasma minocycline levels were below the LLOQ in all 6 subjects receiving vehicle, 9 subjects receiving HY01 1%, and 8 subjects receiving HY01 3%. FIG. 11 shows the mean plasma minocycline concentration for each time point on days 84 and 85.

### Conclusions

The results from the current study demonstrate that a topical minocycline gel is generally safe, well tolerated, and potentially efficacious in the treatment of papulopustular rosacea. Both 1% and 3% concentrations of minocycline significantly decreased the number of inflammatory lesions at week 12 compared to vehicle in a dose dependent manner (p=0.014 and p=0.007, respectively). Notably, this decrease had an early onset of action and durability of effect after cessation of treatment, with significance noted as early as week 4 and persisting through the treatment period and maintaining significance four weeks following cessation of treatment in the 3% treatment arm. In addition to achieving the primary efficacy endpoint, a significantly greater proportion of subjects in the 3% minocycline arm achieved a “clear” or “almost clear” on an IGA (p=0.038).

One potential advantage of topical minocycline gel over oral delivery of the medication is the limited systemic absorption. The current study revealed that the levels of systemic minocycline were very low following the final administration at week 12 in most patients in both treatment arms. It is hypothesized that measuring the serum levels of minocycline at week 12 reflects steady state of minocycline and the multiple time points following a final in-clinic dose therefore reflect a steady state pharmacokinetic profile of the topical minocycline gel.

Oral therapy with minocycline can result in systemic side effects (see, for example, Garner, S. E., et al., Minocyclinefor acne vulgaris: efficacy and safety. Cochrane Database Syst Rev, 2012(8): p. Cd002086.). Gastrointestinal side effects are the most common side effects seen with oral minocycline, while more serious adverse events such as idiopathic intracranial hypertension, hypersensitivity reactions, serum sickness reaction, drug induced lupus-like reaction, pigmentary changes, photosensitivity, candidiasis, and pediatric tooth discoloration are all reported for oral minocycline.

The current study demonstrates very low plasma concentrations of minocycline in both the 1% and 3% minocycline groups, and there were no serious adverse events in either treatment arm. Treatment related adverse events were low in the current study with 3.3% and 5.2% of subjects reporting an event in the 1% and 3% treatment arms, respectively. Overall, the current study demonstrated that topical minocycline gel is safe, efficacious, and well tolerated in papulopustular rosacea patients.

### Example 5—Treatment of Papulopustular Rosacea (PPR) in Human Subjects Using 3% w/w Minocycline Topical Suspension

A multi-center, Phase 3, double-masked, randomized, vehicle-control study is undertaken to study the efficacy and safety of 3% w/w minocycline topical suspension (Test Article) versus Vehicle administered once daily for twelve weeks in subjects with a diagnosis of papulopustular rosacea (PPR). The clinical protocol is described below.

### A. Study Summary

During screening (Day −35 to Day −1), participants will provide written informed consent to participate in the study, and the eligibility of participants will be determined. The investigator or designee will record each participant's demographics and medical history, review concomitant medication to identify any prohibited therapies, and collect vital signs. Study staff will collect blood and urine samples for safety laboratory assessments (serum chemistry, hematology, and urinalysis), and perform a serum pregnancy test in women of childbearing potential (WOCBP). The investigator will perform the 5-point IGA, perform an inflammatory lesion count, and assess the severity of erythema and telangiectasia.

During the Baseline Visit (Day 1), the investigator or designee will repeat the evaluations conducted at the Screening Visit, and perform a physical examination; a urine pregnancy test will be conducted in WOCBP. Safety laboratory assessments will be repeated if the screening assessment occurred >28 days before the Baseline Visit (Day 1). Eligible participants will be randomized in a ratio of 1:1 to receive either Test Article or Vehicle and will be trained in the application of the study drug and completion of a participant diary to record applications of study drug. The investigator will perform the 5-point IGA, perform an inflammatory lesion count, assess the severity of erythema and telangiectasia, complete the Local Application Skin Reaction Scale score, assess skin discoloration scores, and evaluate the hyperpigmentation of the skin to assess the Baseline skin condition. Participants will complete a Rosacea Quality of Life questionnaire (RosaQOL) and a Participant Global Assessment questionnaire.

Study staff will advise participants on the nature of the disease and exacerbating or alleviating environmental factors; they will also instruct participants to minimize exposure to these factors throughout the study. In addition, participants will be advised on the use of the specific cleanser (Pond's Cold Cream) and general precautions related to the study. Randomization and study drug kit number assignment will be performed using an Interactive Web Response System (IWRS). Randomization will be stratified by site and by IGA score (moderate versus severe).

During the 12-week treatment period, participants will apply the study drug once-daily after washing the face with a gentle soap or cleanser, approximately 30 to 60 minutes before retiring at bedtime. Participants will be instructed to wash their entire face with a recommended nighttime cleanser (from a sponsor-provided list) and pat dry with a soft towel before applying the study drug. A thin layer of study drug (approximately 1 gram or 5 fingertip units) will be gently massaged into the entire face at bedtime. A fingertip unit is defined as an amount of study drug that is applied from the distal skin crease to the tip of the index finger (see FIG. 7). Contact with the mouth, eyes, and other mucous membranes should be avoided. The hands should be washed before and after application. Participants will be advised to refrain from washing or wiping their faces for at least 4 hours after the application and to use the recommended cleanser to wash off the study drug in the morning. The participants will also be given a moisturizer with sunscreen to use for protection against outside exposure during daylight hours and encouraged to minimize sun exposure. Artificial UVA/B exposure (tanning beds) is prohibited.

On Day 8, study staff will telephone participants for a preliminary evaluation of safety. They will ask the participant about any AEs experienced during the first week of treatment (including active query of headaches and changes in vision), record any concomitant medications, and confirm that the participant is completing the participant diary.

Participants will return for post-baseline assessment visits at Day 15 (Visit 2), Day 29 (Visit 3), Day 57 (Visit 4), and Day 85 (Visit 5, EOT), returning both used and unused tubes of the study drug at each visit. The investigator will perform the IGA, an inflammatory lesion count, assess the severity of erythema and telangiectasia, and record AEs, local application site reactions, and cosmetic acceptability. Study participants will be asked to complete the Participant Assessment of Tolerability for the treatment and complete the RosaQOL questionnaire. Urine pregnancy testing will be performed for WOCBP at each visit after Screening. Study personnel will review the participant diary at each visit.

Several sites will be selected to participate in photographing participants at Baseline, at Visit 3 (Day 29), and at Visit 5 (Day 85). Participants participating in photography will be required to provide specific consent.

At the end of the treatment period, the Visit 5 (Day 85)/End-of-Treatment (OT) Visit will be performed. Participants terminating the study early will undergo an Early Termination (ET) Visit, to include the same evaluations scheduled for Visit 5 (Day 85)/EOT.

Participants who complete the 12-week treatment period may be eligible to participate in an open-label extension (OLE) study.

Participants who experience an adverse event (AE) during the study will be followed up by telephone at 4 weeks (±3 days) after last treatment application for safety reporting and recording of concomitant medications, unless they enroll in the OLE study.

The objectives and endpoints for the study are presented in the Table below.

TABLE 18
Study Objectives and Endpoints

| OBJECTIVES | ENDPOINTS |
|---|---|
| Primary Efficacy | Primary Efficacy (co-primary endpoints) |
| To evaluate the efficacy of 12-week, once- | IGA success, defined as IGA of “clear” or |
| daily facial application of Test Article in | “almost clear” and at least a 2-grade |
| improving the 5-point Investigator's Global | improvement from Baseline to Week 12 |
| Assessment (IGA) scores from Baseline in | |
| participants with moderate-to-severe PPR | |
| To evaluate the efficacy of 12-week, once- | Absolute change in the inflammatory lesion |
| daily facial application of Test Article as | count (papules and pustules) from |
| assessed by the change in inflammatory lesion | Baseline to Week 12 |
| count from Baseline in participants with | |
| moderate-to-severe PPR | |
| Secondary Efficacy | Secondary Efficacy |
| To evaluate the efficacy of 12-week, once- | Percent change in the inflammatory lesion |
| daily facial application of Test Article as | count (papules and pustules) from |
| assessed by the Erythema Severity Scale and | Baseline to Week 12 |
| the Telangiectasia Severity Scales over the | Absolute and percent change in inflammatory |
| treatment period in participants with | lesion count (papules and pustules) from |
| moderate-to-severe PPR | Baseline to Week 8 |
| | Absolute and percent change in inflammatory |
| | lesion count (papules and pustules) from |
| | Baseline to Week 4 |
| | IGA success, defined as IGA of “clear” or |
| | “almost clear” and at least a 2-grade |
| | improvement from Baseline to Week 8 |
| | IGA success, defined as IGA of “clear” or |
| | “almost clear” and at least a 2-grade |
| | improvement from Baseline to Week 4 |
| | Change in Erythema Severity Scale score |
| | from Baseline to Week 12 |
| | IGA improvement of 2 or more grades |
| | from Baseline to Week 12 |
| | IGA of “clear” or “almost clear” at Week 12 |
| | Change in Erythema Severity Scale |
| | score from Baseline to Week 8 |
| | Change in Erythema Severity Scale |
| | score from Baseline to Week 4 |
| | Change in Telangiectasia Severity Scale |
| | score from Baseline to Week 12 |
| | Change in Telangiectasia Severity Scale |
| | score from Baseline to Week 8 |
| | Change in Telangiectasia Severity Scale |
| | score from Baseline to Week 4 |
| Safety Objectives | Safety Endpoints |
| To evaluate the safety and tolerability of | Occurrence, frequency, and severity of AEs |
| once-daily facial application of Test Article as | by treatment group over the 12-week |
| assessed by the occurrence, frequency, and | treatment period |
| severity of volunteered or observed | |
| adverse events (AEs) | |
| To evaluate the safety and tolerability of | Change in Local Application Skin Reaction |
| once-daily facial application of Test Article as | Scale scores for erythema, dryness, erosion, |
| assessed by local application skin reactions | and edema from Baseline to Week 12 |
| and cosmetic acceptability (skin discoloration | Change in Skin Discoloration Score from |
| and skin pigmentation) | Baseline to Weeks 2, 4, 8, and 12 |
| | Change in Skin Pigmentation Score from |
| | Baseline to Weeks 2, 4, 8, and 12 |
| To evaluate the safety and tolerability of | Change in physical examinations and vital |
| once-daily facial application of Test Article as | signs over the 12-week treatment period |
| assessed by laboratory assessments, physical | Baseline and Visit 5 (Day 85)/ET serum |
| examinations, and vital signs | chemistry, hematology, and urinalysis |
| | laboratory analyses |
| | Participant Assessment of Tolerability |
| | scores at Weeks 2, 4, 8, and 12. |
| Exploratory Objectives | Exploratory Endpoints |
| To evaluate the impact of Test Article on | IGA improvement of 2 or more grades |
| participant-reported outcomes, including | from Baseline to Weeks 4 and 8 |
| quality of life, global assessment, and | IGA improvement of 1 or more grades |
| treatment satisfaction | from Baseline to Weeks 4, 8, and 12 |
| | Rosacea Quality of Life (RosaQoL) |
| | Questionnaire change from |
| | Baseline to Week 12 |
| | Participant Global Assessment |
| | Questionnaire at Week 12 |
| | Participant Treatment Satisfaction |
| | Questionnaire at Week 12 |

### B. Participant Population

The present study will be conducted in adult participants with moderate-to-severe PPR. Participants who do not meet all of the eligibility criteria will not be enrolled. A sufficient number of participants will be enrolled to ensure that at least 540 participants provide data for analysis of the 2 co-primary efficacy endpoints.

### Inclusion Criteria

A participant will be eligible for study participation if he or she meets all of the following criteria:
1. Participant provides written informed consent to participate in the study.
2. Male or non-pregnant female ≥18 years of age with a clinical diagnosis of moderate-to-severe facial PPR, defined as the presence of:
   a. ≥15 and ≤70 facial inflammatory lesions (i.e., papules and pustules), AND
   b. Grade 3 or Grade 4 on the 5-point IGA scale, AND
   c. transient or persistent facial erythema (scored as at least mild on the Erythema Severity Scale), AND
   d. facial telangiectasia (scored as at least mild on the Telangiectasia Severity Scale).
3. Participant is willing to minimize external factors that might trigger PPR flare-ups as recommended per protocol and the participant instructional guide (e.g., spicy foods, thermally hot foods and drinks, hot environments, prolonged sun exposure, strong winds, and alcoholic beverages).
4. Non-nursing women of child-bearing potential (WOCBP) who has a vasectomized partner or has been using an acceptable form of birth control for 2 months prior to study enrollment, and agrees to continue this throughout the study period and for 28 days after last study treatment. Acceptable forms of birth control include:
   a. Total abstinence,
   b. Oral (birth control pills), however WOCBP using oral contraception must be willing to use an appropriate secondary form of contraception during the study,
   c. Intravaginal (e.g., NuvaRing®),
   d. Implantable (e.g., Norplant®),
   e. Injectable (e.g., Depo-Provera®),
   f. Transdermal (e.g., Ortho Evra®),
   g. Intrauterine device, or
   h. Double-barrier (diaphragm or condom with spermicidal gel or foam).
All WOCBP must undergo an in-office serum pregnancy test with a negative result prior to being randomized to receive study drug. In addition, WOCBP must agree to have a urine pregnancy test at each subsequent study visit.
A woman is considered of childbearing potential, i.e. fertile, following menarche and until becoming postmenopausal unless permanently sterile. Permanent sterilization methods include hysterectomy, bilateral salpingectomy and bilateral oophorectomy. A postmenopausal state is defined as no menses for 12 months without an alternative medical cause. Females not of childbearing potential due to menopause must be amenorrheic for ≥1 year.
Male participants must be willing not to attempt to conceive a child during participation in the study, and for at least 28 days after receiving the last application of study treatment; sperm donation is prohibited for the same period.
5. Participants must use the same brand of soap, makeup, hair products, or shaving products for a period of ≥4 weeks prior to the Baseline Visit and agree not to change these product brand/types during the study, with the exception of using the Pond's Cold Cream cleanser and the moisturizer with sunscreen provided by the sponsor.
6. Male participants who are willing to shave, if applicable, at approximately the same time every day and avoid use of skin toners or after-shave.
7. Participants must be willing to minimize exposure to natural sunlight and refrain from the use of artificial UVA/UVB (tanning beds) for the duration of the study.

### Exclusion Criteria

A participant will be excluded from the study if he or she meets any of the following criteria:
1. Presence of any skin condition on the face that would interfere with the diagnosis or assessment of PPR as determined by the investigator.
2. Excessive facial hair (e.g., beards, sideburns, moustaches) that would interfere with the diagnosis or assessment of PPR. Participants also must not grow excess facial hair during the study (i.e., they need to be free of excess facial hair for follow-up visits).
3. History of hypersensitivity or allergy to minocycline, any other tetracycline, or any other component of the formulation or known reactions to cleansers (including Pond's Cold Cream cleanser) and moisturizers with sunscreen.
4. Participants using, or planning to use, concomitant treatments within the 30 days prior to the Baseline Visit (e.g., facial or chemical peels, dermal fillers, acne surgery, intralesional steroids, spironolactone, debridement, cryotherapy, dermabrasion, x-ray, intense pulsed light, laser therapy, or ultraviolet light therapy).
5. Use within 6 months prior to Baseline of oral retinoids (e.g., isotretinoin, acitretin) or therapeutic vitamin A supplements of >10,000 IU/day (multivitamins are allowed).
6. Participants using estrogens or progestin agents (e.g., Gynogen®, Valergen®, Depo Testadiol®, Depogen®, birth control pills) for <2 months prior to the Baseline Visit. (Participants using estrogens for >2 months are not excluded unless the participant expects to change the dose or drug, or discontinue estrogen use during the study).
7. Use of the following within 4 weeks prior to the Baseline Visit:
   a. topical retinoids to the face,
   b. systemic antibiotics known to have an impact on the severity of facial PPR (e.g., tetracycline and its derivatives, erythromycin and its derivatives, sulfamethoxazole, trimethoprim, metronidazole), or
   c. systemic corticosteroids.
8. Use of the following within 2 weeks prior to the Baseline Visit:
   a. topical corticosteroids,
   b. topical antibiotics,
   c. topical medications for PPR (e.g., metronidazole, azelaic acid, erythromycin, ivermectin, sulfur-based topical products),
   d. sauna,
   e. wax epilation of the face.
9. Participants with dermatological and/or other conditions that may be confounded with PPR, and/or require the use of interfering topical or systemic therapy, such as rhinophyma, severe telangiectasia, plaque-like facial edema, rosacea conglobata/fulminans, isolated pustulosis of the chin, perioral dermatitis, acne, seborrheic dermatosis, facial keratosis pillaris, atopic dermatitis, active folliculitis, etc.
10. Participants with sinus tracts or more than 2 nodules.
11. Ocular rosacea (e.g., conjunctivitis, blepharitis, keratitis) of sufficient severity to require topical or systemic antibiotics.
12. Participants with severe erythema, edema, dryness, scaling, stinging/burning, or pruritus.
13. Participants using an investigational drug or participating in an investigational study within 30 days prior to the Baseline Visit. Use of an investigational drug and/or participation in another investigational study is prohibited during this study.
14. Participants who have a history of chronic alcohol or drug abuse within the past year.
15. Medical history of immunodeficiency or other significant ongoing medical condition or disease based on the investigator's judgment.
16. Participants previously treated with minocycline.

### Screen Failures

Screen failures are defined as participants who consent to participate in the clinical study but are not subsequently randomly assigned to a study drug. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure details, eligibility criteria, and any serious adverse events (SAEs). Individuals who do not meet the criteria for participation in this study (screen failure) may be rescreened once if it is anticipated that their status might change.

### Premature Participant Withdrawal

All participants will be advised that they are free to withdraw from participation in this study at any time, for any reason, and without prejudice. The investigator should make every reasonable attempt to keep participants in the study; however, participants must be withdrawn from the study if they withdraw consent to participate. Investigators must attempt to contact participants who fail to attend scheduled visits by telephone or other means to exclude the possibility of an AE being the cause of withdrawal. Should this be the cause, the AE must be documented, reported, and followed as described herein.

The sponsor reserves the right to request the withdrawal of a participant due to protocol deviations or other reasons.

The investigator also has the right to withdraw participants from the study at any time for lack of therapeutic effect that is intolerable or otherwise unacceptable to the participant, for intolerable or unacceptable AEs, intercurrent illness, noncompliance with study procedures, administrative reasons, or in the investigator's opinion, to protect the participant's best interest.

If a participant is withdrawn before completing the study, the reason for withdrawal and the date of discontinuation will be recorded on the appropriate page of the electronic case report form (eCRF). Whenever possible and reasonable, the evaluations that were to be conducted at the completion of the study should be performed at the time of premature discontinuation.

### Discontinuation of Study Intervention

A participant may voluntarily discontinue treatment at any time and for any reason without prejudice to his or her future medical care. The investigator, sponsor, or medical monitor may also discontinue a participant from treatment at any time if it is medically necessary or in the interest of the participant's safety. Additional reasons for premature discontinuation of the study drug may include AEs and major noncompliance with the study procedures, as described below. The withdrawal of a participant from study drug by the investigator will be discussed with the medical monitor before the participant stops study drug, whenever possible.

A participant will be discontinued from treatment if any of the following criteria are met:
Participant withdraws consent.
In the opinion of the investigator, medical monitor, or sponsor, it is not in the participant's best interests to continue treatment.
Occurrence of an AE or serious AE (SAE) that, in the investigator's opinion, warrants discontinuation of treatment.
Pregnancy as informed by the participant or as determined by a positive urine pregnancy test.
The participant is deemed to be a treatment failure, which is defined as the worsening of condition and requirement for alternate or supplemental therapy for the treatment of rosacea during the study.

Participants who discontinue treatment due to a lack of treatment effect after completing at least 8 weeks of treatment will be included in the ITT and PP populations as treatment failures, and the change in inflammatory lesion count from the Baseline Visit to the last completed visit prior to discontinuation due to lack of efficacy will be carried forward in the primary endpoint analysis. Participants who discontinue treatment for other reasons will be excluded from the PP population.

If a participant discontinues prematurely, the investigator will perform the Early Termination (ET) Visit, capturing the reason for discontinuation and performing the assessments of the EOT Visit.

Participant Discontinuation/Withdrawal from the Study

A participant will be discontinued from this study if any of the following criteria are met:
Participant withdraws consent.
In the opinion of the investigator, medical monitor, or sponsor, it is not in the participant's best interests to continue in the study.
Occurrence of an AE or SAE that, in the opinion of the investigator, warrants discontinuation of the participant from the study.
Pregnancy as informed by the participant or as determined by a positive urine pregnancy test.
Significant noncompliance with study procedures that would interfere with the study results or increase the participant's risks in the study as determined by the investigator.
The participant is deemed to be a treatment failure, which is defined as a worsening of condition and requirement for alternate or supplemental therapy for the treatment of rosacea during the study.
A participant may withdraw from the study at any time at his/her own request or may be withdrawn at any time at the discretion of the investigator for safety, behavioral, compliance, or administrative reasons.

If a participant withdraws from the study, he/she may request destruction of any samples taken and not tested, and the investigator must document this in the study center study records.

Participants whose condition worsens and who require alternate or supplemental therapy for the treatment of PPR during the study will be discontinued, and the ET Visit will be performed.

If a participant does not return for scheduled visits, every effort will be made to contact the participant and document the EOT Visit assessments. The investigator must document the primary reason for the study participant's discontinuation in the source document and on the appropriate eCRF.

Randomized participants who discontinue due to an AE will have all events documented and followed to satisfactory resolution.

If a participant who does not meet enrollment criteria is inadvertently enrolled, the investigator must consult with the medical monitor and sponsor to determine if that participant must be discontinued from study treatment.

### Lost to Follow-Up

A participant will be considered lost to follow-up if he or she repeatedly fails to return for scheduled visits and the study center is unable to contact him/her. The following actions must be taken if a participant fails to return to the clinic for a required study visit:
The study center must attempt to contact the participant, reschedule the missed visit as soon as possible, counsel the participant on the importance of maintaining the assigned visit schedule, and ascertain whether or not the participant wishes to and/or should continue in the study.
Before a participant is deemed lost to follow-up, the investigator or designee must make every effort to regain contact with the participant (where possible, 3 telephone calls and, if necessary, a certified letter to the participant's last known mailing address or local equivalent methods). These contact attempts will be documented in the participant's medical record.
If the participant continues to be unreachable, he/she will be considered to have withdrawn from the study.

### C. Treatments

Study treatment are described below.

### Identification of Investigational Products

The study drug includes the Test Article (3% Topical Minocycline Gel) and Vehicle. Study drug will be supplied as matching 30 g yellow gel in aluminum tubes with a nasal tip and a plastic cap closure labeled for a blinded study.

TABLE 19
Study Drug Details

| | Test Article | |
|---|---|---|
| Study Drug Name: | (3% Topical Minocycline Gel) | Vehicle |
| Active Ingredient: | Minocycline | None |
| Unit Dose Strength(s)/ | 3% w/w | 0% |
| Dosage Level(s): | | |
| Route of Administration | Topical | Topical |
| Dosing Instructions: | Approximately 1 g (i.e., 5 | Approximately 1 g (i.e., 5 |
| | fingertip units) daily at bedtime | fingertip units) daily at bedtime |
| Packaging and Labeling | Yellow gel 30 g in aluminum tubes | Yellow gel 30 g in aluminum tubes |
| | with a nasal tip and a plastic cap | with a nasal tip and a plastic cap |
| | closure labeled for a blinded study | closure labeled for a blinded study |

Composition of the Test Article (3% Topical Minocycline Gel) is described in Table 20. Composition of the Vehicle is described in Table 21. Particles of minocycline in the topical suspension that is the Test Article desirably have a D90 particle size within the range of from 3 microns to 4 microns, and a D50 particle size within the range of from 1 to 2 microns.

TABLE 20
Test Article (3% Minocycline Gel)

| Component | Amount | Function |
|---|---|---|
| Minocycline base | 3% w/w | active ingredient |
| Polymeric hydrocarbon gelling agent | 67.9% w/w | gelling agent |
| commercially available under the | | |
| tradename VERSAGEL ® M-750 | | |
| Mineral oil | 29.1% w/w | wetting agent |

TABLE 21
Composition of Vehicle

| Component | Amount | Function |
|---|---|---|
| Polymeric hydrocarbon | 70.0% w/w | gelling agent |
| gelling agent commercially | | |
| available under the | | |
| tradename VERSAGEL ® M-750 | | |
| Mineral oil | 30.0% w/w | wetting agent |

The minocycline base was minocycline base in crystalline Form II. The polymeric hydrocarbon gelling agent commercially available under the tradename VERSAGEL® M-750 was a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol).

Each participant will be instructed by the investigator or designated staff on proper application technique. The investigator/staff will demonstrate the appropriate amount required for 1 application, which is equal to approximately 1 g (i.e., 5 fingertip units) of the study drug product. The sponsor-supplied moisturizer with sunscreen will be used for demonstration of the application during the training session.

The study drug will be applied once-daily at bedtime over a 12-week treatment period. Participants will be instructed to wash their entire faces with a recommended nighttime cleanser (see below), and pat dry with a soft towel before applying the study drug. A thin layer of study drug (approximately 1 g or 5 fingertip units) will be gently massaged into the entire face at bedtime. A fingertip unit is defined as an amount of study drug that is applied from the distal skin crease to the tip of the index finger (see FIG. 7). Contact with the mouth, eyes, and other mucous membranes should be avoided. The hands should be washed following application. Participants will be advised to refrain from washing or wiping their faces for at least 4 hours after the application and to use a study cleanser to wash off the study drug in the morning; participants will be provided with Pond's Cold Cream Cleanser to use in the morning to remove the study drug. The participants will be given a moisturizer with sunscreen to use for protection against outside exposure during daylight hours.

The following application instructions will be provided to the participant:
1. At bedtime, before applying the study product, wash entire face with warm water (NOT HOT) using a mild cleanser or soap from the list of recommended nighttime cleanser products. Pat face dry with soft towel.
2. Squeeze 1 application on index fingertip equivalent to approximately 1 inch in length.
You will need 5 fingertip applications in total.
3. Apply the study drug to 5 separate sections on the face (1 squeeze or fingertip per section): the left and right temples, left and right cheeks, and chin.
4. Gently massage study drug into the skin on the entire face. Avoid contact with eyes, mouth and the inside of the nose. After application, wash hands thoroughly with mild soap. Leave the study drug on the face overnight or for at least 4 hours.
5. Wash the study drug off in the morning, or no less than 4 hours after application, using the provided Pond's Cold Cream cleanser.

Participants will be instructed to:
Protect their faces with the provided moisturizer with sunscreen when spending time in the sun.
Wash their hands prior and following the investigational drug application procedures.
Avoid exposure to extreme cold weather conditions, when possible, to avoid rosacea flare-ups.
Limit intake of spicy food and alcohol throughout the duration of the study.

Two tubes of study drug will be dispensed to the participant at the Baseline Visit. The tubes will be weighed, and the weight of each tube will be recorded (to the nearest 0.1 g) prior to dispensing to the participant. The participant will be required to return both tubes of study drug at each visit, and the study site will weigh and record the weight (to the nearest 0.1 g) of each tube at each visit. Two additional tubes of study drug will be dispensed to the participant at Day 15, Day 29, and Day 57 to ensure the participant has an adequate supply of study drug at all times. The site will use IWRS system for study drug dispensation.

The first application of study drug will be performed at the study site at the Baseline Visit (Day 1) under the supervision of the investigator/staff. Subsequent applications will be performed at bedtime as directed.

### Other Treatments Administered

Participants will be provided with a cold cream cleanser and with a moisturizing sunscreen for use throughout their participation in the study. In addition, participants should use their usual nighttime cleanser before applying the study drug, from a list of approved products; the list will be included in the participant instruction leaflet.

### Cleanser—Pond's Cold Cream

Participants will be provided with Pond's Cold Cream cleanser, to be used for the removal of minocycline or Vehicle each morning, as described herein. The cleanser should not be used for removal of the study drug until at least 4 hours has passed since application. Pond's Cold Cream will be provided to study sites by the sponsor, for distribution to participants.

Moisturizer with Sunscreen

Participants will be provided with a moisturizer that contains sunscreen for use when spending time in the sun, as described herein. Participants are required to agree to refrain from sunbathing, using sun tanning booths/beds, or excessive exposure to the sun for the duration of the study. Moisturizer with sunscreen will be provided to study sites by the sponsor, for distribution to participants.

### Recommended Nighttime Cleanser

In addition to being provided with Pond's Cold Cream cleanser (for removing the study drug in the morning) and a moisturizing sunscreen, participants will also be required to wash their face using a recommended nighttime cleanser before applying the study drug at bedtime. The sponsor will provide a list of recommended nighttime cleansers, which will be included with the participant instruction leaflet.

In accordance with the inclusion criteria, participants should avoid changing their brand of soap for a period of ≥4 weeks prior to the Baseline Visit and agree not to change these product brand/types during the study. Participants whose regular cleanser is not included in the sponsor's list of recommended products will be required to switch to a recommended product during the screening period; the newly adopted product should be used for at least 7 days before the participant attends the Baseline Visit.

### Dispensing and Storage

Study participants will be dispensed a tube of the study drug at Day 1, Day 15 (±4 days), Day 29 (±4 days), and Day 57 (±4 days). At the Baseline Visit (Day 1), participants will receive the randomized treatment along with a participant diary, and written instructions and precautions; participants will also receive Pond's Cold Cream cleanser and a moisturizer with sunscreen (or similar product) at this visit.

The study drug storage temperature is 20° C. to 25° C. (68° F. to 77° F.), protected from light and moisture; excursions are permitted between 15° C. and 30° C. (59° F. to 86° F.). All study drug must be stored in a secure, environmentally controlled, and monitored (manual or automated) area in accordance with the labeled storage conditions with access limited to the investigator and authorized study center staff.

### Treatment Compliance

### Compliance

The prescribed dosage, timing, and mode of administration may not be changed. Any departures from the intended regimen must be recorded in the eCRFs.

At each visit following the Baseline Visit (Day 1) and prior to dispensing study drug, previously dispensed study drug will be retrieved by the investigator and compliance assessed. Participants exhibiting poor compliance based on participant diary completion and by the weight of the gel tubes should be counseled on the importance of good compliance to the study dosing regimen. Compliant participants are defined as those who have completed 75% of the treatment applications and have not missed more than 5 consecutive days of dosing.

### Participant Diaries

During the Baseline Visit (Day 1), participants will be provided with a diary in which to record their study drug applications, and will be instructed on its use.

Throughout the treatment period, participants will be asked to record their daily application of the study drug in the diary. Deviations from the planned doses (missed dose or timing) will be recorded on the subject's eCRF. Study personnel will review diaries at each visit, and diaries (or downloads from diaries, as applicable) will be collected as source documents. Information from participant diaries will be transcribed on the appropriate eCRF pages for documentation of participant compliance with the study drug. Any missed application notations will be clarified with the participant and documented.

Study staff will contact participants by telephone on Day 8 and confirm that the participant diary is being completed. At each study visit, study staff will review the participant diary, and retrain the participants on diary completion as necessary.

### Treatment Groups

### Method of Assigning Participants to Treatment Groups

All participants will be centrally assigned to randomized study treatment using an IWRS. Before the study is initiated, the login information and directions for the IWRS will be provided to each study center. The randomization scheme will be generated and kept strictly confidential throughout the conduct of the study and will not be available to the sponsor, the investigator, the study staff, or the clinical staff who could have an impact on the outcome of the study.

Randomization will be performed at the time the participant has met all eligibility criteria and is ready for enrollment. Eligible participants will be randomized in a ratio of 1:1 to receive either minocycline or Vehicle. The IWRS will assign participants to the treatment arm and specify the kit number to be dispensed. Randomization will be stratified by investigative site and inflammatory lesion count.

### Blinding and Unblinding Treatment Assignment

This is a double-blind study with limited access to the randomization code. The investigational treatment and vehicle will be identical in physical appearance. The treatment each participant receives will not be disclosed to the investigator, study center staff, participant, sponsor, or study vendors. The treatment codes will be held by the sponsor's designated biostatistician. Overall unblinding will take place at the end of the study only after database lock has been achieved.

The IWRS will be programmed with blind-breaking instructions. In case of an emergency, the investigator has the sole responsibility for determining if unblinding of a participant's treatment assignment is warranted. Participant safety must always be the first consideration in making such a determination. If the investigator decides that unblinding is warranted, the investigator should make every effort to contact the sponsor prior to unblinding a participant's treatment assignment unless this could delay emergency treatment of the participant. If a participant's treatment assignment is unblinded, the sponsor must be notified within 24 hours after breaking the blind. The date and reason that the blind was broken must be recorded in the source documentation and eCRF, as applicable.

The blinding scheme for each study drug product will be available to the United States (US) Food and Drug Administration (FDA) or other regulatory agency investigators at the time of site inspection to allow for verification of the treatment identity of each participant.

The blinding code will not be broken except in emergency situations for which the identification of the study drug of a participant is required by the investigator to complete an SAE report. In such situations, the medical monitor or the investigator will use the IWRS to unblind the treatment for the individual participant. Unblinded information will be held by designated individual(s), and the date and reason for breaking the blind will be recorded.

The medical monitor will be contacted by telephone prior to unblinding but no later than 24 hours after unblinding. As the study is blinded, the investigator will promptly document and explain to the sponsor any premature unblinding (e.g., accidental unblinding, unblinding due to an SAE) of the investigational study products.

### Permitted and Prohibited Therapies

All concomitant medications used (including over-the-counter medications and herbal supplements) will be recorded in the source document and on the appropriate eCRF. Information about concomitant medications will be collected at Screening and at all subsequent visits.

Any medication or vaccine (including over-the-counter or prescription medicines, vitamins, and/or herbal supplements) that the participant is receiving at the time of enrollment (within 30 days before the time of enrollment) or receives during the study must be recorded on the eCRF along with the following information: reason for use, dates of administration including start and end dates, and dosage information including dose and frequency. The medical monitor should be contacted if there are any questions regarding concomitant or prior therapy.

### Permitted Therapies

Other concomitant medications are allowed (excluding the prohibited medications listed herein), but should be limited to those medications considered necessary.

Participants will be provided with a cleanser for removing the study drug, and a moisturizer with sunscreen, as described herein. Standardization of these treatments is expected to reduce intersubject variability in PPR assessments due to differences in cleansing and sun-protection behaviors. Further efforts to reduce potential for cosmetic products to impact on efficacy assessments are described herein.

### Prohibited Therapies

The use of the following prescription and over-the-counter drug products are prohibited during study participation:
Oral retinoids (e.g., isotretinoin, acitretin) or therapeutic vitamin A supplements ≥10,000 units/day (multivitamins are allowed) for 6 months prior to the Baseline Visit
Treatment with estrogens or progestin agents (e.g., Gynogen®, Valergen®, Depo Testadiol®, Depogen®, birth control pills) for <2 months prior to the Baseline Visit. (Participants using birth control pills for ≥2 months are not excluded unless the participant expects to change dose, drug, or discontinue birth control pill use during the study)
Concomitant treatment with facial or chemical peels, dermal fillers, acne surgery, intralesional steroids, spironolactone, debridement, cryotherapy, dermabrasion, X-ray, IPL, laser therapy, or UV therapy within 30 days of Baseline and throughout the duration of the study
Topical retinoids to the face within 4 weeks prior to the Baseline Visit and throughout the duration of the study
Systemic and topical antibiotics known to have an impact on the severity of facial PPR (e.g., tetracycline and its derivatives, erythromycin and its derivatives, sulfamethoxazole, trimethoprim, metronidazole) for 4 weeks prior to Baseline and throughout the duration of the study
Systemic corticosteroids for 4 weeks prior to Baseline and throughout the duration of the study
Topical corticosteroids for 2 weeks prior to Baseline and throughout the duration of the study
Topical antibiotics for 2 weeks prior to Baseline and throughout the duration of the study
Topical medications for PPR (e.g., metronidazole, azelaic acid, erythromycin, ivermectin, sulfur-based topical products) for 2 weeks prior to the Baseline Visit and throughout the duration of the study
Change in moisturizers, new brands of makeup, creams, lotions, or powders other than the assigned treatment to the treatment area throughout the duration of the study
Occlusive dressings or wrappings should be avoided in treatment areas throughout the duration of the study

If the investigator becomes aware that a participant has taken a prohibited medication during the study, he/she will report the incident to the medical monitor within 48 hours; the medical monitor and/or sponsor will provide written approval of the participant's continuation or discontinuation from the study.

### Restrictions

Participants must use the same brand of soap, makeup, hair products, or shaving products for a period of ≥4 weeks prior to the Baseline Visit. In addition, they must agree not to change these product brand/types during the study with the exception of using the Pond's Cold Cream cleanser and the moisturizer with sunscreen provided by the sponsor.

During the 12-week treatment period, participants will wash their face with a recommended nighttime cleanser before applying the study drug. To wash their face, participants should use a gentle soap of cleanser from a list provided by the sponsor, maintaining their current skin care regimen. Participants whose regular cleanser is not included in the sponsor's list of recommended products will be required to switch to a recommended product during the screening period; the newly adopted product should be used for at least 7 days before the participant attends the Baseline Visit.

Male participants who shave must do so at approximately the same time every day. Participants also must not grow excess facial hair during the study (i.e., they need to be free of excess facial hair for follow up visits). All participants should refrain from the use of wax epilation.

Additional restrictions include the following. Participants must refrain from sunbathing, using sun tanning booths/beds, or excessive exposure to the sun for the duration of the study. Participants must refrain from using saunas for the duration of the study. When possible, participants must avoid exposure to extreme cold weather conditions to avoid rosacea flare-ups. The only meal and dietary restrictions are that participants must limit their intake of spicy food and thermally hot foods and drinks throughout the duration of the study.

At each visit, investigators will remind participants to minimize external factors that might trigger PPR flare-ups (e.g., spicy foods, thermally hot foods and drinks, hot environments, prolonged sun exposure, strong winds, and alcoholic beverages). During the treatment period, investigators will ask participants about any noteworthy exposures to such triggers.

Treatment after End of Study

After the end of the study, participants may be able to enroll in an OLE study to receive minocycline (3%). Participant not enrolled in this OLE study will be treated according to standard clinical practice. Participants who discontinue from the study and are not eligible for the OLE study will receive a follow-up telephone call 28 days after their last treatment if they have experienced an AE during the study. Participants who did not experience an AE will not receive a follow-up call.

### D. Assessments

Investigators will be trained on the completion of investigator assessments (e.g., IGA, Erythema Severity Scale, Telangiectasia Severity Scale, Local Application Skin Reaction Scale, Skin Discoloration Score, and Hyperpigmentation Score) to ensure that completion is standardized across the different study sites.

### Efficacy Variables

Efficacy variables required for evaluation of the co-primary endpoints will be the first assessments performed. The investigator will perform the IGA assessment first, followed by the lesion count.

### Investigator's Global Assessment (IGA)

An IGA will be determined according to the visit schedule. This scale should not be a reflection of treatment response but should describe the condition at each visit. Therefore, no reference should be made to Baseline in the evaluation.

TABLE 22
Investigator's Global Assessment (IGA) Scale

| Score | Grade | Definition |
|---|---|---|
| 0 | Clear | No inflammatory lesions present |
| 1 | Almost Clear | Very few small papules/pustules |
| 2 | Mild | Several small papules/pustules |
| 3 | Moderate | Several small and large papules/pustules |
| 4 | Severe | Numerous small and large papules/ |
| | | pustules, up to 2 nodules |

### Inflammatory Lesion Count

The area of lesion count assessment is defined as the face from the jaw line to the hair line. The lesion counts will be performed according to the visit schedule. The lesion counts will be performed as follows:
1. Participants will wash their faces and remove all makeup at least 15 minutes prior to assessment. Male participants must shave before coming for the study visit.
2. Participants will be seated at arm's length (or slightly closer) from the assessor performing the lesion counts and at the same height as the assessor. Participants will not be seated in direct sunlight.
3. The room will have a balanced and consistent artificial light source or natural light from a North-facing window.
4. The investigator or approved designee in this study will be instructed to perform all lesion counts. It is essential that the same assessor performs all lesion counts for a given participant for the duration of that participant's participation in the study.
5. Lesions on each area of the face are to be considered separately. Lesions will be counted just below the hair line and no lower than the mandibular line (jaw line). The face will be systematically scanned from left to right, and the lesions will be counted and recorded.

All lesions counted will be palpated. If a lesion is not palpable, it will be considered nonactive (emerging or resolving). The inflammatory lesion count will include the total count of papules and pustules, which are defined as follows:
PAPULE—A type of inflammatory lesion; a small erythematous, palpable lesion, usually solid and <0.5 cm in diameter.
PUSTULE—A type of inflammatory lesion; a small palpable lesion with an erythematous base <0.5 cm in size and containing pus or yellow-white liquid.
NODULE—A type of inflammatory lesion; a large palpable erythematous papule or plaque that is >0.5 cm in diameter.

Papules will be counted separately from pustules (inflammatory lesions). All lesion types will be counted separately for each quadrant on the face. Counts of nodules will be reported separately and not included in the inflammatory lesion counts.

### Erythema Severity Scale

The investigator will grade the severity of nontransient facial erythema from 0 to 4 as described in the Table below. Although inflammation (papules, pustules, plaques) or dry appearance may obscure the level of erythema, underlying redness should be evaluated disregarding this effect. Inflammation or dry appearance may be noted, but perilesional erythema will not be included in this assessment. Transient erythema (flushing) will also not be included.

TABLE 23
Erythema Severity Scale

| Score | Grade | Definition |
|---|---|---|
| 0 | Absent | No perceptible erythema. |
| 1 | Mild | Slight erythema (light pink) |
| | | with either restricted central |
| | | involvement or generalized whole face. |
| 2 | Moderate | Definite erythema (red) with |
| | | either restricted central |
| | | involvement or generalized whole face. |
| 3 | Severe | Pronounced erythema (marked redness) |
| | | with either restricted central |
| | | involvement or generalized whole face. |
| 4 | Very Severe | Severe erythema (dark red-purple hue) |
| | | with either restricted central involvement |
| | | or generalized whole face. |

### Telangiectasia Severity Scale

The investigator will grade facial telangiectasia from 0 to 4 using the Telangiectasia Severity Scale in the following Table.

TABLE 24
Telangiectasia Severity Scale

| Score | Grade | Definition |
|---|---|---|
| 0 | Absent | No telangiectasia |
| 1 | Mild | Slight telangiectasia characterized |
| | | by a few fine, small red vessels <0.2 mm |
| | | or less in diameter; telangiectasia |
| | | covers less than 10% of face |
| 2 | Moderate | Pronounced telangiectasia characterized |
| | | by several fine vessels and/or by a few |
| | | large vessels ≥0.2 mm in diameter; |
| | | telangiectasia covers between 10% and 30% of face |
| 3 | Severe | Severe telangiectasia characterized by |
| | | many fine vessels or large vessels; |
| | | telangiectasia covers more than 30% of face |
| 4 | Very Severe | Very severe telangiectasia characterized |
| | | by many large vessels and fine vessels; |
| | | telangiectasia covering at least 30% of face |

### Safety Variables

Safety assessments will include the evaluation of AEs, clinical laboratory assessments (including pregnancy tests), vital signs, physical examinations, investigator assessment of local application skin reactions, the Skin Discoloration Score, evaluation of hyperpigmentation of the skin, and participant assessment of tolerability. The planned time points for all safety assessments are provided in the visit schedule.

### Adverse Events

The classifications, descriptions, and management of AEs, and any special considerations for AEs, are provided herein. Adverse events will be reported by the participant (or, when appropriate, by a caregiver, surrogate, or the participant's legally authorized representative).

The investigator and any designees are responsible for detecting, documenting, and recording events that meet the definition of an AE or SAE and remain responsible for following up AEs that are serious, considered related to the study drug or study procedures, or that caused the participant to discontinue treatment with the study drug. In addition to reports of AEs by nonleading questioning from investigators and spontaneous participant reporting, investigators will also actively question participants about whether or not they have experienced any headaches or changes in vision.

### Local Application Skin Reaction Scale

At the visits listed in the visit schedule, the investigator will score any erythema, dryness, erosion, and edema thought to be secondary to the medication on a scale of 0-3, where 0=absent, 1=mild (slight, barely perceptible), 2=moderate (distinct presence), 3=severe (marked, intense).

### Skin Discoloration Score

At the visits listed in the visit schedule, the investigator will assess skin discoloration (staining) using the 6 point ordinal scale in the Table below. The purpose of the skin discoloration scale is to identify discoloration secondary to the minocycline active product, which has a yellow tint. Post-inflammatory pigment change, actinic damage, and ethnic pigmentation are all to be ignored for the purpose of this rating.

TABLE 25
Skin Discoloration Score

| Score | Grade | Definition |
|---|---|---|
| 0 | None | No discoloration |
| 1 | Minimal | Minimal discoloration of the skin, |
| | | barely perceptible |
| 2 | Slight | Slight, but definite discoloration |
| | | of the skin |
| 3 | Moderate | Moderate discoloration of the skin, |
| | | clearly perceptible at a glance |
| 4 | Pronounced | Pronounced discoloration of the skin |
| | | over much of the application site |
| 5 | Very | Very pronounced, strong discoloration |
| | Pronounced | of the skin over the whole |
| | | application site |

### Hyperpigmentation of the Skin

At the visits listed in the visit schedule, the investigator will assess hyperpigmentation of the skin using the scale in the Table below. Every effort will be made to have participants evaluated by the same investigator throughout the study.

TABLE 26
Skin Pigmentation Score

| Score | Grade | Definition |
|---|---|---|
| 0 | None | No abnormal pigmentation |
| 1 | Minimal | Minimal hyperpigmentation of the skin, |
| | | barely perceptible |
| 2 | Slight | Slight, but definite hyperpigmentation |
| | | of the skin |
| 3 | Moderate | Moderate hyperpigmentation of the skin, |
| | | clearly perceptible at a glance |
| 4 | Pronounced | Pronounced hyperpigmentation of the skin |
| | | over much of the application site |
| 5 | Very | Very pronounced, strong hyperpigmentation |
| | Pronounced | of the skin over the whole |
| | | application site |

### Physical Examination & Vital Signs

Physical examinations will include height (at Screening only), weight, and an evaluation of organs and systems (general appearance, heart/cardiovascular, lungs, ears/nose/throat, extremities, gastrointestinal, and skin). A participant with abnormalities other than the presence of rosacea lesions that, in the opinion of the investigator, may pose a risk to his or her safety or may interfere with the assessment of safety or efficacy in the study will be excluded.

Vital signs will be assessed by the investigator according to the visit schedule. Assessments will include body temperature, heart rate, and blood pressure (systolic and diastolic). Blood pressure and pulse rate will be measured after the participant has been sitting restfully for at least 5 minutes.

### Prior and Concomitant Medication Review

All medications (both prescription and nonprescription, and including vitamins, herbals, topicals, inhaled, and intranasal) taken within 30 days prior to the start of the study drug and through the final study visit will be recorded on the appropriate eCRF (using generic names, if known) with the corresponding indication, start dates, and stop dates. Previous treatment of rosacea must be recorded irrespective of the duration it was given. At each study visit, participants will be asked whether they have started or discontinued any medication since their previous study visit. This includes single-use or as needed (PRN) medication use. The corresponding condition for each concomitant medication will be captured in the participant's Medical History or in the AE eCRF for medications starting during the study.

### Clinical Laboratory Safety Assessments

Blood and urine will be collected from participants at Screening and at Visit 5, or at ET Visits. Additional clinical laboratory assessments will be performed at Baseline (Day 1, Visit 1) only for those participants whose screening laboratory assessments were conducted >28 days before the Baseline Visit.

The chemistry analysis will include glucose, uric acid, calcium, sodium, potassium, chloride, alkaline phosphatase, total bilirubin, bicarbonate, creatinine, blood urea nitrogen (BUN), aspartate aminotransferase (AST), alanine transaminase (ALT), lactic acid dehydrogenase (LDH), total protein, albumin, and C-reactive protein. Participants will not be asked to fast prior to specimen collection.

Hematology evaluation will be completed with a complete blood count (CBC). Urinalysis will include testing the leukocyte esterase, nitrite, pH, protein, blood, specific gravity, ketones, glucose, and bilirubin in each sample. Blood and urine samples for hematology, serum chemistry, and urinalysis will be sent to a central laboratory for analysis. Pregnancy tests (serum test at Screening Visit, urine tests at subsequent visits) will be conducted at the study sites.

### Participant-Reported Outcomes

### Quality of Life Assessment

The Rosacea Quality of Life Questionnaire (RosaQOL) will be completed at the Baseline Visit and at each follow-up visit. The RosaQOL is a self-administered questionnaire, comprising the items shown in the Table below. The participant will respond to each item with a score from 1-5, where 1=Never, 2=Rarely, 3=Sometimes, 4=Often, 5=All the Time.

TABLE 27
Rosacea Quality of Life Assessment Items

| | Hypothesized |
|---|---|
| RosaQoL item | Construct |
| I worry that my rosacea may be serious | Emotion |
| My rosacea burns or stings | Symptom |
| I worry about getting scars from my rosacea | Emotion |
| I worry that my rosacea may get worse | Emotion |
| I worry about side effects from rosacea medications | Emotion |
| My rosacea is irritated | Symptom |
| I am embarrassed by my rosacea | Emotion |
| I am frustrated by my rosacea | Emotion |
| My rosacea makes my skin sensitive | Symptom |
| I am annoyed by my rosacea | Emotion |
| I am bothered by the appearance of my skin | Emotion |
| (redness, blotchiness) | |
| My rosacea makes me feel self-conscious | Emotion |
| I try to cover up my rosacea (with makeup) | Functioning |
| I am bothered by persistence/reoccurrence of my rosacea | Emotion |
| I avoid certain foods or drinks because of my rosacea | Functioning |
| My skin feels bumpy (uneven, not smooth, irregular) | Symptom |
| My skin flushes | Symptom |
| My skin gets irritated easily (cosmetics, aftershaves, | Symptom |
| cleansers) | |
| My eyes bother me (feel dry or gritty) | Symptom |
| I think about my rosacea | Emotion |
| I avoid certain environments (heat, humidity, cold) | Functioning |
| because of my rosacea | |

### Participant Treatment Satisfaction Questionnaire and Participant Global Assessment Questionnaire

The Participant Treatment Satisfaction Questionnaire will be completed at the EOT/ET Visit (Day 85). The Participant Treatment Satisfaction Questionnaire is a self-administered questionnaire, comprising the first 3 items shown in the Table below.

The Participant Global Assessment Questionnaire will be completed with the Participant Treatment Satisfaction Questionnaire at the EOT/ET Visit (Day 85), and comprises a single question—the final question in the questionnaire shown in the Table below.

TABLE 28
Participant Treatment Satisfaction Questionnaire

| | | | | |
|---|---|---|---|---|
| Overall satisfaction | □ 1-Excellent | □ 2-Good | □ 3-Fair | □ 4-Poor |
| of the treatment's | (very satisfied) | (moderately | (slightly | (not satisfied |
| tolerability | | satisfied) | satisfied) | at all) |
| Was the face | □ 1-Excellent | □ 2-Good | □ 3-Fair | □ 4-Poor |
| cleansing procedure | (very satisfied) | (moderately | (slightly | (not satisfied |
| easy to perform | | satisfied) | satisfied) | at all) |
| Was the cleansing | □ 1-Excellent | □ 2-Good | □ 3-Fair | □ 4-Poor |
| procedure able to | (very satisfied) | (moderately | (slightly | (not satisfied |
| remove the study | | satisfied) | satisfied) | at all) |
| product from the face | | | | |
| Did you like the product | □ 1-Yes | □ 2-No | | |

### Participant Assessment of Tolerability

Participants will be asked to assess the tolerability of the medication and the answer will be recorded in the questionnaire below.

TABLE 29
Local Skin Reaction Questionnaire

| | | | | |
|---|---|---|---|---|
| Presence of itching | □ 0-None | □ 1-Mild | □ 2-Moderate | □ 3-Severe |
| Presence of burning | □ 0-None | □ 1-Mild | □ 2-Moderate | □ 3-Severe |
| Presence of pain | □ 0-None | □ 1-Mild | □ 2-Moderate | □ 3-Severe |
| Presence of skin discoloration | □ 0-None | □ 1-Mild | □ 2-Moderate | □ 3-Severe |

### Photography

Photography will be performed at selected study sites only. Participants who are unwilling to participate in the photography will be allowed to opt out of this procedure. Participants who agree to participate in photography must provide specific written informed consent for this procedure before photographs are collected. Photographs will be taken at the Baseline Visit (Visit 1) and at the participant's EOT/ET Visit (Visit 5). Sites selected for participation in photography will be provided with standardized photographic equipment and trained on how to obtain accuracy and consistency of procedures.

### E. Adverse Events

### Descriptions

### Adverse Events

An Adverse Event (AE) is any untoward medical occurrence in a patient or clinical investigation subject administered a pharmaceutical product that does not necessarily have a causal relationship with this treatment. An AE can therefore be any unfavorable and unintended sign (including an abnormal laboratory finding, for example), symptom, or disease temporally associated with the use of a medicinal product, whether or not related to the medicinal product.

Pre-existing diseases or conditions will not be considered AEs unless there is an increase in the frequency or severity, or a change in the quality, of the disease or condition (worsening of a pre-existing condition is considered an AE.)

Events that occur in participants treated with control product (Vehicle gel), or during treatment free periods of the study, are also considered AEs.

### Adverse Drug Reaction

All noxious and unintended responses to an investigational product related to any dose should be considered adverse drug reactions (ADRs). The phrase “responses to an investigational product” means that a causal relationship between an investigational product and an AE is at least a reasonable possibility, i.e., the relationship cannot be ruled out. All AEs judged by either the reporting investigator or the sponsor as having a reasonable causal relationship to a study drug qualify as ADRs.

All AEs for which the judgment of relationship to study drug is “possible” or higher will be considered ADRs. If a relationship to study drug is not provided, then the AE must be treated as if it were “possible.”

### Unexpected Adverse Event/Adverse Drug Reaction

An expected AE or ADR is one for which the nature or severity is consistent with the known AE profile of the product. For a pre-approval test product, the known information is contained in the Investigator's Brochure (IB). For a marketed product, the known information is contained in the current package insert for the product.

An unexpected adverse event (UAE) or unexpected adverse drug reaction (UADR) is one for which the nature or severity of which is not consistent with the applicable product information (e.g., IB for an unapproved investigational product or package insert/summary of product characteristics for an approved product). For example, hepatic necrosis would be unexpected (greater severity) if the IB only listed elevated hepatic enzymes or hepatitis. Likewise, cerebral thromboembolism and cerebral vasculitis would be unexpected (greater specificity) if the IB only listed cerebral vascular accidents.

Furthermore, reports that add significant information on specificity or severity of a known, already documented adverse reaction constitute unexpected events. Examples would be (a) acute renal failure as an expected adverse reaction with a subsequent new occurrence of interstitial nephritis (interstitial nephritis would be unexpected) and (b) hepatitis with a first occurrence of fulminant hepatitis (fulminant hepatitis would be unexpected.)

### Serious Adverse Events/Drug Reaction

A Serious Adverse Event (SAE) is any untoward medical occurrence that at any dose:
results in death
is life-threatening (NOTE: The term “life-threatening” in the definition of “serious” refers to an event in which the subject was at risk of death at the time of the event; it does not refer to an event that hypothetically might have caused death if it were more severe.)
requires inpatient hospitalization or prolongation of existing hospitalization (NOTE: Inpatient hospitalization is defined as 24 hours in a hospital or an overnight stay. An elective hospital admission to treat a condition present before exposure to the study drug, or a hospital admission for a diagnostic evaluation of an AE, does not qualify the condition or event as an SAE. Further, an overnight stay in the hospital that is only due to transportation, organization, or accommodation problems and without medical background does not need to be considered an SAE.)
results in persistent or significant disability/incapacity
is a congenital anomaly (NOTE: A congenital anomaly in an infant born to a mother who was exposed to the study drug during pregnancy is an SAE. However, a newly diagnosed pregnancy in a subject that has received a study drug is not considered an SAE unless it is suspected that the study drug(s) interacted with a contraceptive method and led to the pregnancy.)
is an important medical event (NOTE: Medical and scientific judgment should be exercised in deciding whether it is appropriate to consider other situations serious, such as important medical events that may not be immediately life-threatening or result in death or hospitalization but may jeopardize the subject or may require intervention to prevent one of the other outcomes listed in the definition above. Examples of such events are intensive treatment in an emergency room or at home for allergic bronchospasm, blood dyscrasias, or convulsions that do not result in hospitalization, development of drug dependency, or drug abuse. The occurrence of malignant tumors is also to be considered serious.)

### Significant Adverse Events

Other significant AEs are defined as marked hematological and other laboratory abnormalities (other than those meeting the definition of serious) and any events that led to an intervention, including withdrawal of test drug, dose reduction, or significant additional concomitant therapy.

### Treatment-Emergent Adverse Events

An AE is defined as treatment emergent if the first onset or worsening is after the first administration of study drug (minocycline topical gel or Vehicle) and not more than 30 days after the last administration of study drug.

### Event Assessment and Follow-Up of Adverse Events

The occurrence of an AE or SAE may come to the attention of study personnel during study visits and interviews of a study participant presenting for medical care or upon review by a study monitor.

All AEs including local and systemic reactions not meeting the criteria for SAEs will be captured on the appropriate eCRF. Information to be collected includes event description, time of onset, clinician's assessment of severity, relationship to study product (assessed only by those with the training and authority to make a diagnosis), and time of resolution/stabilization of the event. All AEs occurring while on study must be documented appropriately regardless of relationship. All AEs will be followed to adequate resolution.

Any medical condition that is present at the time that the participant is screened will be considered as baseline and not reported as an AE. However, if the study participant's condition deteriorates at any time during the study, it will be recorded as an AE.

Changes in the severity of an AE will be documented to allow an assessment of the duration of the event at each level of severity to be performed. Any AEs characterized as intermittent require documentation of onset and duration of each episode.

At each study visit, the investigator will inquire about the occurrence of AE/SAEs since the last visit. The investigator will record all reportable events with start dates occurring any time after informed consent is obtained until 7 days (for non-serious AEs) or 28 days (for SAEs) after the participant's last application of study drug. Events will be followed for outcome information until resolution or stabilization.

Participants who terminate early due to an AE will continue to be monitored for 28 days from last treatment for AEs, unless they withdraw consent or are lost to follow-up.

### Assessment

The investigator is responsible for the detection and documentation of events meeting the criteria and definition of an AE or SAE described previously. At each visit, the participant will be allowed time to spontaneously report any issues since the last visit or evaluation. The investigator will then monitor and/or ask about or evaluate AEs using nonleading questions, such as, “How are you feeling?”, “Have you experienced any issues since your last visit?”, or “Have you taken any new medications since your last visit?”.

Any clinically relevant observations made during the visit will also be considered AEs.

In addition to spontaneous reports of AEs and AEs reported after the investigator's nonleading questions, investigators will also actively query participants with regard to the incidence of headaches and changes in vision. Investigators should not conduct these active queries until AFTER nonleading questioning has been used to elicit AE reporting.

### Evaluation

The clinical severity of an AE will be classified as follows:
Mild—Usually transient and may require only minimal treatment or therapeutic intervention. The event does not generally interfere with usual activities of daily living.
Moderate—Usually alleviated with additional specific therapeutic intervention. The event interferes with usual activities of daily living, causing discomfort but poses no significant or permanent risk of harm to the participant.
Severe—Interrupts usual activities of daily living, or significantly affects clinical status, or may require intensive therapeutic intervention.

It is important to distinguish between severe AEs and SAEs. Severity is a classification of intensity, whereas an SAE is an AE that meets serious criteria, as described herein.

Actions taken may consist of the following:
Dose not changed—An indication that a medication schedule was maintained.
Dose reduced—An indication that a medication schedule was modified by subtraction, either by changing the frequency, strength, or amount.
Drug interrupted—An indication that a medication schedule was modified by temporarily terminating a prescribed regimen of medication.
Drug withdrawn—An indication that a medication schedule was modified through termination of a prescribed regimen of medication.
Not applicable—Determination of a value is not relevant in the current context.
Unknown—Not known, not observed, not recorded, or refused.

The outcome at the time of last observation will be classified as follows:
Recovered/resolved
Recovered/resolved with sequelae
Recovering/resolving
Not recovered/not resolved
Fatal*
Unknown
   Fatal should only be selected as an outcome when the AE results in death. If more than one AE is judged to be possibly related to the participant's death, the outcome of death should be indicated for each such AE. Although “fatal” is usually an event outcome, events such as sudden death or unexplained death should be reported as SAEs.

The investigator must make an assessment of each AE's relationship to the study drug. The categories for classifying the investigator's opinion of the relationship are as follows:
Probably related—“Probably related” conveys that there are facts, evidence, and/or arguments to suggest a causal relationship, rather than a relationship cannot be ruled out.
Possibly related—“Possibly related” suggests that the association of the AE with the study treatment is unknown; however, the AE is not reasonably supported by other conditions.
Unlikely to be related—“Unlikely to be related” suggests that only a remote connection exists between the study treatment and the AE. Other conditions, including chronic illness, progression or expression of the disease state or reaction to concomitant therapy, appear to explain the reported AE.
Unrelated—“Unrelated” is used if there is not a reasonable possibility that the study treatment caused the AE.
Unknown—All efforts should be made to classify the AE according to the above categories. The category “unknown” (unable to judge) may be used only if the causality is not assessable, eg, because of insufficient evidence, conflicting evidence, conflicting data, or poor documentation.
   The AE relationship to the study drug must be assessed separately by the investigator and the sponsor.

### Documentation

All AEs that occur within the period of observation for the study must be documented in the eCRF with the following information, where appropriate:
AE name or term
When the AE first occurred (start date and time)
When the AE stopped (stop date and time or an indication of “ongoing”)
Severity of the AE
Seriousness (hospitalization, death, etc.)
Actions taken
Outcome
Investigator opinion regarding the AE relationship to the study drug(s)
   Adverse events documented following active query about headaches or changes in vision will be reported separately from spontaneous AE reports, in a specific eCRF page.

### Treatment of Adverse Events

Adverse events that occur during the study will be treated, if necessary, by established standards of care. If such treatment constitutes a deviation from the protocol, the participant may continue in the study at the discretion of the investigator. The decision about whether the participant may continue in the study will be made by the sponsor after consultation with the investigator and/or medical monitor.

If AEs occur in a participant that are not tolerable, the investigator must decide whether to stop the participant's involvement in the study and/or treat the participant. Special procedures may be recommended for the specific study drug, such as the collection of a serum sample for determining blood concentrations of study drug, specific tapering procedures, or treatment regimens, as appropriate.

For double-blinded studies, it is not necessary to unblind a participant's treatment assignment in most circumstances, even if an SAE has occurred. If unblinding is necessary, the unblinding procedures described herein should be followed.

### Follow-Up

Any AE will be followed up to 28 days after the last dose of study drug to a satisfactory resolution, or until it becomes stable, or until it can be explained by another known cause(s) (i.e., concurrent condition or medication) and clinical judgment indicates that further evaluation is not warranted. All findings relevant to the final outcome of an AE must be reported in the participant's medical record and recorded on the eCRF page.

### Pregnancy

All WOCBP who participate in the study should be counseled on the need to practice adequate birth control and on the importance of avoiding pregnancy during study participation. Participants are required to have been using an acceptable form of birth control for 2 months prior to study enrollment and for 28 days after the last application of the study treatment. Participants should be instructed that if pregnancy occurs or is suspected (in female participants or in the partner of male participants), they should inform the investigator or study staff immediately.

Pregnancy testing will be conducted prior to administration of the study drug on every woman of childbearing potential. A woman who is found to be pregnant at the Screening Visit will be excluded from the study and considered to be a screening failure. A woman who becomes pregnant during study drug treatment or within 28 days of discontinuing the study drug will be immediately discontinued from study participation.

### F. Statistics

### Statistical Analysis

The Statistical Analysis Plan (SAP) will be developed and finalized before database lock. This section is a summary of the planned statistical analyses of the primary and secondary endpoints.

Descriptive statistical methods will be used to summarize the data from this study with confidence intervals (CIs) calculated for the primary and secondary efficacy endpoints. Unless stated otherwise, the term “descriptive statistics” refers to the number of participants (n), mean, median, SD, minimum, and maximum for continuous data; and frequencies and proportions for categorical data. All data collected during the study will be included in data listings.

For the purpose of using site as a covariate, the sites contributing with a low number of randomized participants will be pooled with a comparable site based on geographical location or other characteristics. The approach for pooling will be defined in the SAP before unblinding.

P-values of less than 0.05 will be considered statistically significant based on a 2-sided test unless otherwise specified. All statistical analyses will be conducted with the SAS® System, version 9.3 or higher.

### Analysis Populations

The following 3 analysis populations are planned for this study:
Intent-to-treat (ITT): All randomized participants
Per-Protocol (PP): All randomized participants with at least one application of study drug with no key protocol deviations during the study that would affect the interpretation of the primary efficacy assessments. The PP population will be determined prior to database lock and unblinding of treatment assignment. Key protocol deviations will be described further in the SAP
Safety: All randomized participants who are treated with at least 1 application of study drug

Inclusion in the analysis populations will be determined prior to database lock. If a participant is randomized incorrectly or is administered the incorrect study drug, analyses of the ITT population will be based on the assigned treatment whereas all other analyses will be based on the actual treatment.

The statistical analysis will be based on the ITT, PP, and Safety populations. The Safety population will be used to analyze the safety endpoints, and the ITT population will be used to analyze all efficacy endpoints. The PP population will be used to analyze the primary efficacy endpoints. The ITT population will be considered to be the primary population for the efficacy analyses.

### Efficacy Analysis

### Statistical Hypotheses

To attain the primary efficacy objective of HY01 3% to be considered superior to Vehicle, both co-primary efficacy endpoints must be significant (i.e., attain significance at the 2 sided 0.05 level). The null hypotheses of the equality of HY01 and Vehicle are as follows:
H₀₁: The changes from Baseline in inflammatory lesion count at Week 12 in the 2 treatment groups are equal.
H₀₂: The IGA success rates at Week 12 are equal in the 2 treatment groups, with IGA success defined as IGA of “clear” or “almost clear” and at least a 2-grade improvement from Baseline.

The study level Type I error of 5% will be controlled using a Fixed-Sequence method. Hypotheses testing for the secondary efficacy endpoints will only be conducted if both co-primary efficacy endpoints have demonstrated statistical significance at the 0.05 level. Testing of the secondary endpoints will be performed sequentially in the prespecified order provided herein.

### Efficacy Endpoints

The co-primary efficacy endpoints are:
IGA success, defined as IGA of “clear” or “almost clear” and at least a 2-grade improvement from Baseline to Week 12
Absolute change in the inflammatory lesion count (papules and pustules) from Baseline to Week 12

The secondary efficacy endpoints are:
Percent change in the inflammatory lesion count (papules and pustules) from Baseline to Week 12
Absolute and percent change in inflammatory lesion count (papules and pustules) from Baseline to Week 8
Absolute and percent change in inflammatory lesion count (papules and pustules) from Baseline to Week 4
IGA success, defined as IGA of “clear” or “almost clear” and at least a 2-grade improvement from Baseline to Week 8
IGA success, defined as IGA of “clear” or “almost clear” and at least a 2-grade improvement from Baseline to Week 4
Change in Erythema Severity Scale score from Baseline to Week 12
IGA improvement of 2 or more grades from Baseline to Week 12
IGA of “clear” or “almost clear” at Week 12
Change in Erythema Severity Scale score from Baseline to Week 8
Change in Erythema Severity Scale score from Baseline to Week 4
Change in Telangiectasia Severity Scale score from Baseline to Week 12
Change in Telangiectasia Severity Scale score from Baseline to Week 8
Change in Telangiectasia Severity Scale score from Baseline to Week 4

The exploratory efficacy endpoints are:
IGA improvement of 2 or more grades from Baseline to Weeks 4 and 8
IGA improvement of 1 or more grades from Baseline to Weeks 4, 8, and 12
RosaQoL Questionnaire change from Baseline to Week 12
Participant Global Assessment Questionnaire at Week 12
Participant Treatment Satisfaction Questionnaire at Week 12

### Primary Analysis

The ITT and PP analysis populations will be used to analyze the primary efficacy endpoints. The ITT population will be considered to be the primary population for the efficacy analyses.

Inflammatory Lesion Count: For the primary analysis of change from Baseline in inflammatory lesion count at Week 12, and for secondary analyses at time points prior to Week 12, a mixed-model repeated measures (MMRM) analysis will be performed. The model will include treatment, pooled investigational site, Baseline lesion count, Baseline IGA group (=2; ≥3), visit, and visit-by-treatment interaction as explanatory variables. Model-based point estimates (i.e., least squares [LS] means), 95% CIs, and P-values will be reported. An unstructured covariance structure will be used to model the within-subject errors. In the event that the covariance structure does not converge, additional structures will be considered. Further details will be given in the SAP. A sensitivity analysis of the primary analysis will be performed on the PP population. Further sensitivity analyses on the primary endpoint using such methods as Markov Chain Monte Carlo (MCMC) to explore the impact of missing data will be described in a detailed SAP. Descriptive summaries (such as mean, standard deviation, median, minimum, and maximum) will also be provided for the ITT and PP populations. Results from the MMRM primary analysis will be used to determine study success.

Investigator's Global Assessments: For the dichotomized IGA score, success will be defined as an improvement to a score of “clear” or “almost clear” and at least a 2-grade improvement from Baseline. The dichotomized IGA score at Week 12, as well as secondary analyses at time points prior to Week 12, a generalized estimating equation (GEE) model with treatment, pooled investigational site, Baseline lesion count, Baseline IGA group (=2; ≥3), visit, and visit-by-treatment interaction as explanatory variables. Odds ratios, 95% CIs, and P-values will be reported. An unstructured covariance structure will be used to model the within-subject errors. In the event that the covariance structure does not converge, additional structures will be considered. Further details will be given in the SAP. A sensitivity analysis on the primary endpoint using such methods as fully conditional specification logistic regression method to explore the impact of missing data will be described in the SAP. Descriptive summaries (such as frequency and percentage of the responders at each visit) will also be provided. Results from the GEE primary analysis will be used to determine study success.

### Secondary Analyses

Analyses of secondary endpoints will be carried out for the ITT population. Sensitivity analyses will not be done for the secondary endpoints listed below.

Inflammatory Lesion Count: Percent change in the inflammatory (papules and pustules) lesion count from the Baseline to Week 12 will be analyzed using MMRM with factors for treatment, pooled investigational site, Baseline lesion count, Baseline IGA group (=2; ≥3), visit, and visit-by-treatment interaction, following the same method as for the primary endpoint.

Erythema Severity Scale Score and Telangiectasia Severity Scale Score: Change in the erythema and Telangiectasia Severity Scale scores from Baseline will be analyzed using MMRM with factors for treatment, pooled investigational site, Baseline score, Baseline IGA group (=2; ≥3), visit, and visit-by-treatment interaction, following the same method as for the primary endpoint.

IGA Improvement: The IGA improvement of 2 or more from Baseline will be analyzed using GEE with factors for treatment, pooled investigational site, baseline lesion count, baseline IGA group (=2; ≥3), visit, and visit-by-treatment interaction, following the same method as for the primary endpoint.

IGA of “clear” or “almost clear” at Week 12: The IGA of “clear” or “almost clear” will be analyzed using GEE with factors for treatment, pooled investigational site, baseline lesion count, baseline IGA group (=2; ≥3), visit, and visit by treatment interaction, following the same method as for the primary endpoint.

### Exploratory, Corroborative, Sensitivity, and Other Analyses

IGA Improvement: The IGA improvement of 1 or more from Baseline will be analyzed using GEE with factors for treatment, pooled investigational site, baseline lesion count, baseline IGA group (=2; ≥3), visit, and visit-by-treatment interaction, following the same method as for the primary endpoint.

Quality of Life Assessment: The RosaQOL response to each item will be averaged overall and by subgroup (i.e. Emotion, Functional, and Symptom). The absolute change from Baseline will then be analyzed using an MMRM with factors for treatment, pooled investigational site, Baseline score, Baseline IGA group (=2; ≥3), visit, and visit-by-treatment interaction as explanatory variables, following the same method as the primary endpoint.

Participant Global Assessment Questionnaire: The Participant Global Assessment questionnaire will be summarized descriptively by visit.

Participant Treatment Satisfaction Questionnaire: The Participant Treatment Satisfaction questionnaire will be summarized descriptively by visit.

### Safety and Tolerability Analyses

All safety analyses will be performed on the Safety population. The variables for safety endpoints are TEAEs, change in local skin reaction scale scores, change in discoloration score, change in skin pigmentation score, change in physical examination, change in vital signs, baseline and Visit 5 (Day 85)/ET serum chemistry, hematology, and urinalysis, and Participant Assessment of Tolerability scores.

### Adverse Events

Treatment-emergent AEs are defined as AEs that first occur or worsen in severity after the first administration of study drug and prior to 30 days after the last administration of study drug. Adverse events will be coded using the Medical Dictionary for Regulatory Activities (MedDRA). For each treatment group, the numbers of TEAEs and incidence rates will be tabulated by preferred term and System Organ Class (SOC).

Treatment-emergent AEs will be summarized for each treatment group based on frequency, maximum severity, relationship to study drug, SAEs, TEAEs leading to death, and TEAEs leading to discontinuation of study drug. Commonly occurring TEAEs, i.e., those that occur in 5% or more of the participants in either treatment group, will be summarized using descriptive statistics.

### Clinical Laboratory Evaluations

All serum chemistry, hematology, and urinalysis measurements will be summarized for each treatment group using descriptive statistics at baseline and the terminal visit for raw numbers, change from Baseline, and conversion from normal to abnormal. The incidence of treatment-emergent abnormal values will also be summarized using descriptive statistics.

### Vital Signs, Weight, and Pregnancy Test Results

All vital signs and weight measurements and pregnancy test results will be summarized for each treatment group using descriptive statistics at each visit for raw numbers and change from Baseline. The incidence of treatment-emergent abnormal vital sign values will also be summarized using descriptive statistics.

### Safety Scales and Additional Local Assessments

Tolerability Assessments: Tolerability assessments will include local skin reaction scores for erythema, dryness, burning/stinging, erosion, edema, pain, itching, and bleeding and will be summarized by severity and treatment group. Tolerability scores will be summarized using descriptive statistics.

Cosmetic Acceptability: Cosmetic acceptability will be evaluated based on hyperpigmentation of the skin at each visit and skin discoloration scores at Week 12 compared to Baseline. Hyperpigmentation scores will be summarized using descriptive statistics.

### Example 6—Improving Skin Epidermal Barrier in Human Subjects with Inflammatory Rosacea Using 3% w/w Minocycline Topical Suspension

The ability of minocycline topical suspension to improve the skin epidermal barrier in human subjects suffering from inflammatory rosacea was evaluated according to the clinical study described below, in which minocycline topical suspension was administered to the face of the patient once daily in the evening, and the patient was periodically evaluated for improvement. Experimental procedures and results are described below.

### Part I—Experimental Procedures

### A. Study Summary

A clinical study was performed to evaluate ability of 3% w/w minocycline topical suspension to improve the skin epidermal barrier in human subjects suffering from inflammatory rosacea. Male and female subjects (at least 18 years of age) were enrolled in this single site monadic study to evaluate the effect of 3% w/w minocycline topical suspension on skin barrier function. Subjects who signed informed consent forms and met all inclusion criteria and none of the exclusion criteria were enrolled at the baseline visit. Thirty-one subjects were enrolled in the study.

Efficacy was evaluated by measuring transepidermal water loss (TEWL), where statistically significant decrease in TEWL measurements after 4 weeks of once-daily Test Article use indicates improvement in skin epidermal barrier. A single one-minute TEWL measurement was taken from a target site on the left cheek at baseline, day 1, week 2, and week 4 after reaching steady state. Subjects equilibrated 20-30 minutes at the research center prior to the measurement.

Corneometry was also used to evaluate efficacy, where a statistically significant increase in corneometry measurements after 4 weeks of once-daily Test Article use indicates improvement in skin epidermal barrier. Triplicate pin probe corneometry measurements were taken from a target site on the right cheek with the average recorded after the subject has equilibrated to the environment of the research center for 20-30 minutes at baseline, day 1, week 2, and week 4.

Along with descriptive statistics (means and percentages), numerical TEWL and corneometry data were analyzed using a Student's t-test. Change was considered significant at a p value of less than or equal to 0.05.

### B. Investigational Product (IP)

The Test Article was 3% w/w minocycline topical suspension, which was supplied as a yellow gel in an aluminum tube with a nasal tip and a plastic cap closure. Particles of minocycline in the Test Article had a D90 particle size within the range of from 3 microns to 4 microns, and a D50 particle size within the range of from 1 to 2 microns.

TABLE 30
Composition of Test Article (3% w/w
minocycline topical suspension)

| Component | Amount | | Function |
|---|---|---|---|
| Minocycline base | 3% | w/w | active ingredient |
| Polymeric hydrocarbon | 67.9% | w/w | gelling agent |
| gelling agent commercially | | | |
| available under the | | | |
| tradename VERSAGEL ® M-750 | | | |
| Mineral oil | 29.1% | w/w | wetting agent |

The minocycline base was minocycline base in crystalline Form II. The polymeric hydrocarbon gelling agent commercially available under the tradename VERSAGEL® M-750 was a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) was present in an amount within the range of 2.5% to 10% (w/w), the butylene-ethylene-styrene copolymer (e.g., weight-average molecular weight of about 100,000 g/mol) was present in an amount within the range of 0.1% to 2.5% (w/w), the butylated-hydroxytoluene was present in an amount <0.5% (w/w), and the remainder was mineral oil (e.g., having a weight-average molecular weight in the range of 230-700 g/mol).

### C. Study Design

Male and female subjects were enrolled in this single-site monadic study to evaluate the effect of a topical 3% minocycline gel on skin barrier function. Subjects who signed consent and met all inclusion criteria and none of the exclusion criteria were enrolled at the baseline visit. Subjects were asked to continue their self-selected cleanser, which they had used without difficulty for the prior 30 days, for the 4 week duration of the study unchanged. Subjects also continued all facial products unchanged. Subjects were not to use other oral or topical rosacea products. Subjects were washed out of all topical rosacea products for at least 2 weeks and all oral rosacea products for at least 4 weeks. Subjects of childbearing potential underwent a urine pregnancy test.

At the baseline visit (day 0), subjects acclimated to the environment of the research center for 20-30 minutes. Following the acclimation period, the subjects underwent a one-minute transepidermal water loss (TEWL) measurement on the left cheek. In addition, triplicate pin probe corneometry measurements were taken from the right cheek with the average recorded. Subjects with moderate rosacea (15+ inflammatory papules and/or pustules) were dispensed the Test Article to apply every evening to their entire clean, washed face. The Test Article was provided as a single tube of approximately 30 g. Subjects recorded their applications in a compliance diary. A brief medical history and concomitant medications review was conducted. Subjects of childbearing potential underwent a urine pregnancy test.

Subjects returned to the research center at day 1. Subjects acclimated to the environment of the research center for 20-30 minutes. Following the acclimation period, the subjects underwent a one-minute transepidermal water loss (TEWL) measurement on the left cheek. In addition, triplicate pin probe corneometry measurements were taken from the right cheek with the average recorded. Subjects' Test Article was checked for proper use and adequacy of remaining product. Subjects of childbearing potential underwent a urine pregnancy test. Subject diaries were checked for compliance.

Subjects returned to the research center at week 2. Subjects acclimated to the environment of the research center for 20-30 minutes. Following the acclimation period, the subjects underwent a one-minute transepidermal water loss (TEWL) measurement on the left cheek. In addition, triplicate pin probe corneometry measurements were taken from the right cheek with the average recorded. Subjects' Test Article was checked for proper use and adequacy of remaining product. Subjects of childbearing potential underwent a urine pregnancy test. Subject diaries were checked for compliance.

Subjects returned to the research center at week 4. Following the acclimation period, the subjects underwent a one-minute transepidermal water loss (TEWL) measurement on the left cheek. In addition, triplicate pin probe corneometry measurements were taken from the right cheek with the average recorded. A brief medical history and concomitant medications review was conducted. Subjects of child bearing potential underwent a urine pregnancy test. Subject Test Article and diaries were checked for compliance and all study materials were collected. Subjects completed their study participation.

Thirty-one subjects were enrolled in the study. All subjects were at least 18 years of age.

### D. Study Eligibility Criteria

Subjects who satisfied all of the following inclusion criteria and had none of the following exclusion criteria were allowed to enroll in the study. Inclusion criteria for the study were:
1. Subjects with moderate facial rosacea (15+ inflammatory facial lesions).
2. Male or female subjects age 18+ years of age.
3. Subjects with all Fitzpatrick skin types.
4. Subjects of all complexion types (normal, oily, dry, combination).
5. Subjects who had used the same cleanser without difficulty for 30 days and were willing continue using the same cleanser during the study.
6. Subjects who agreed during the study not to introduce any new colored cosmetics (lipsticks, eye shadows, facial foundations, blush, powder). No other rosacea products or moisturizers were applied to the face for the duration of the study.
7. Subjects with no known medical conditions that, in the investigator's opinion, may have interfered with study participation.
8. Women of childbearing potential were willing to use a form of birth control during the study. For the purpose of this study, the following were considered acceptable methods of birth control: oral contraceptives, the levonorgestrel implant commercially available as NORPLANT®, medroxyprogesterone acetate injection commercially available as DEPO-PROVERA®, double barrier methods (e.g., condom and spermicide) and abstinence.
9. Subjects signed an Informed Consent Form in compliance with 21CFR Part 50: “Protection of Human Subjects.”
10. Subjects were dependable, able to follow directions, and willing to comply with the schedule of visits.
11. Subjects were in generally good physical and mental health.

The exclusion criteria for the study were:
1. Any dermatological disorder that, in the investigator's opinion, may have interfered with the accurate evaluation of the subject's skin characteristics, except for the study condition of rosacea.
2. Subjects who were not willing to apply the assigned study product to their face as instructed or not willing to use the same self-selected cleanser during the study.
3. Subjects who had used any topical prescription or OTC rosacea products for 2 weeks prior to study entry.
4. Subjects who had taken any oral prescription rosacea medications for 4 weeks prior to study entry.
5. Subjects who had any facial treatments in the past 6 months or were not willing to withhold all facial treatments during the course of the study. Facial treatments include facials, facial peels, photo facials, laser treatments, dermabrasion, botulinum toxin (Botox), injectable filler treatments, intense pulsed light (IPL), acid treatments, tightening treatments, facial plastic surgery, etc.
6. Subjects who were pregnant, breast feeding, or planning a pregnancy.
7. Subjects with clinically significant unstable medical disorders.
8. Subjects who were unwilling or unable to comply with the requirements of the protocol.
9. Subjects who had history of a psychological illness or condition that would interfere with their ability to understand and follow the requirements of the study.
10. Subjects who were participating in any other clinical trial at the time.

### Concomitant Medications

All oral and topical prescription medications remained unchanged during the study. No oral or topical over the counter or prescription rosacea medications were allowed.

### E. Efficacy Analysis

Efficacy analysis included transepidermal water loss (TEWL) analysis and corneometry analysis. Along with descriptive statistics (means and percentages), numerical TEWL and corneometry data was analyzed using a Student's t-test. Significance was defined at the p<0.05 level based on a two-sided test.

### Transepidermal Water Loss (TEWL) Measurements

A single one-minute TEWL measurement was taken from a target site on the left cheek at baseline, day 1, week 2, and week 4 after reaching steady state. Subjects equilibrated 20-30 minutes at the research center prior to the measurement.

### Corneometry Measurements

Triplicate pin probe corneometry measurements were taken from a target site on the right cheek with the average recorded after the subject had equilibrated to the environment of the research center for 20-30 minutes at baseline, day 1, week 2, and week 4.

### Part II—Results

Results of the clinical study are described below. Thirty subjects successfully completed the trial. One subject withdrew from the trial at week 2 because he did not like the feel of the Test Article on his skin. No adverse experiences, adverse events, or serious adverse events occurred during the conduct of the trial.

Corneometry results are presented in Table 31 below. Corneometry is a measure of the water content of the skin. The average of three readings from the check was recorded and analyzed as longitudinally as change from baseline. A higher measurement is indicative of increased skin water content. A DermaLab pin probe corneometer was used to obtain the measurements (Cortex Technologies, Hasund, Denmark). The pin probe was pressed into the left cheek at the designated target site around the subject's facial mask, due to the COVID-19 pandemic present during the administration of the study, to obtain a touch reading. The Test Article consistently produced an average 20% increase in skin water content at all evaluation time points. There was a 23% (p=0.003) increase at day 1, a 22% (p=0.003) increase at week 2, and a 20% increase (p=0.001) at week 4. These findings indicate the Test Article produced no irritation in subjects with rosacea-induced sensitive skin and functioned as a moisturizer to increase skin hydration.

TABLE 31
Corneometry Results

| Wilcoxon | | | | |
|---|---|---|---|---|
| Rank-Sign | | | | |
| two tailed | | | | |
| paired | Baseline | Day 1 | Week 2 | Week 4 |
| Mean A | 114.68 | 141.57 | 139.50 | 137.37 |
| Baseline A | | 114.68 | 114.68 | 114.68 |
| % Change | | 23% | 22% | 20% |
| A v. Baseline p= | | 0.003 | 0.003 | 0.001 |

TEWL results are presented in Table 32 below. TEWL measurements were obtained from the cheek of all subjects. TEWL is a measurement of the water vapor leaving the skin and represents a skin barrier assessment. A lower reading is indicative of better skin barrier function. An RG TEWL unit (Cyberderm, Broomall, Pa.) was used to obtain the TEWL reading from the target site identified on the right cheek around the subject's mask, worn due to the COVID-19 pandemic present during the administration of the study. The probe was placed gently on the face and held in place to collect measurements for one minute. Over the last 5 seconds of sampling, the average reading was recorded once steady state had been reached. Persons with the inflammatory condition of rosacea possess a barrier defect due to the presence of the skin disease. There was a cumulative decrease in TEWL over the duration of the study, indicating a progressive improvement in skin barrier function. There was an 11% reduction in TEWL at day 1, an 18% reduction in TEWL at week 2 (p=0.001), and a 28% decrease in TEWL at week 4 (p<0.001). This improvement in skin barrier is believed to be due to a combination of skin healing and the moisturizing properties of the Test Article.

TABLE 32
Transepidermal Water Loss (TEWL) Results

| Wilcoxon | | | | |
|---|---|---|---|---|
| Rank-Sign | | | | |
| two tailed | | | | |
| paired | Baseline | Day 1 | Week 2 | Week 4 |
| Mean A | 10.33 | 9.18 | 8.47 | 7.43 |
| Baseline A | | 10.33 | 10.33 | 10.33 |
| % Change | | −11% | −18% | −28% |
| A v. Baseline p= | | 0.208 | 0.001 | <0.001 |

### Summary

Test Article applied once daily contributed to skin moisturization. This benefit was directionally observed after the first application, and by week 4, skin barrier improvement was highly statistically significant. More specifically, the TEWL endpoint was a statistically significant decrease in TEWL measurements after 4 weeks of Test Article use indicating skin barrier improvement, and the TEWL endpoint was met with a highly statistically significant (p<0.001) decrease in TEWL of 28% after 4 weeks of medication use. The corneometry endpoint was a statistically significant increase in corneometry measurements after 4 weeks of Test Article use indicating skin barrier improvement, and the corneometry endpoint was met with a statistically significant (p=0.001) 20% increase in skin water content after 4 weeks of medication use.

### Example 7—Rheological Evaluation of a Minocycline Topical Suspension

A minocycline topical suspension was subjected to rheological evaluation. Experimental procedures and results are described below.

### Part I—Experimental Procedures

The following rheological tests were conducted on a sample of the 1% Test Article described in Table 33 below: Shear Rate Sweep, Sear Stress Sweep, and Oscillation Stress Sweep.

TABLE 33
Composition of 1% Test Article

| Component | Amount | | Function |
|---|---|---|---|
| Minocycline base | 1% | w/w | active ingredient |
| Polymeric hydrocarbon | 69.3% | w/w | gelling agent |
| gelling agent commercially | | | |
| available under the | | | |
| tradename VERSAGEL ® M-750 | | | |
| Mineral oil | 29.7% | w/w | wetting agent |

The minocycline base was minocycline base in crystalline Form II. The polymeric hydrocarbon gelling agent commercially available under the tradename VERSAGEL® M-750 is a mixture of ethylene-propylene-styrene copolymer, butylene-ethylene-styrene copolymer, butylated-hydroxytoluene, and mineral oil. The ethylene-propylene-styrene copolymer (e.g., weight-average molecular weight of about 200,000 g/mol) is present in an amount within the range of 2.5% to 10% (w/w), the butylene-ethylene-styrene copolymer (e.g., weight-average molecular weight of about 100,000 g/mol) is present in an amount within the range of 0.1% to 2.5% (w/w), the butylated-hydroxytoluene is present in an amount <0.5% (w/w), and the remainder is mineral oil (e.g., having a weight-average molecular weight in the range of 230-700 g/mol).

### Shear Rate Sweep

The Shear Rate Sweep analysis was performed as follows: following a 30 s equilibration time at 34° C., the sample was exposed to a 30 s pre-shear at a rate of 1 s⁻¹ before leading directly into a shear rate sweep, 1.0 s⁻¹ to 1000 s⁻¹, logarithmically scaled, 6 points per decade of shear rate, shear applied for 30 s at each rate with viscosity calculated over the final 5 seconds of each step.

### Shear Stress Sweep

The Shear Stress Sweep analysis was performed as follows: following a 60 s equilibration time at 25° C., the sample was subjected to a shear stress sweep from 0.1 Pa to 100 Pa, logarithmically spaced, 8 points per decade of shear stress. Steady-state sensing was employed to ensure individual viscosity readings reached an acceptable degree of elastic or thixotropic equilibrium before being recorded. At each step of the test, viscosity was monitored every 5 seconds. Viscosity was recorded only when 3 successive measurements were within 5% of each other. A 60 s timeout was set: if an equilibrium viscosity was not achieved after that time the viscosity at that instant was recorded regardless of degree of equilibrium.

### Oscillation Stress Sweep

The Oscillation Stress Sweep analysis was performed as follows: following a 60 s equilibration time at 25° C., the sample was exposed to an oscillatory stress sweep ranging from 0.1 Pa to 1000 Pa, 10 points per decade, at 1 Hz oscillation frequency. A step termination was set such that if at any point the oscillation strain exceeded 1500%, the test would immediately end.

### Part II—Results

TABLE 34
Controlled Rate Viscosity Data

| | Viscosity at 1s⁻¹ (Pa · s) | | | Viscosity at 1000s⁻¹ (Pa · s) | | |
|---|---|---|---|---|---|---|
| Sample | Run 1 | Run 2 | Mean | Run 1 | Run 2 | Mean |
| 1% Test Article | 30.7 | 30.2 | 30.5 | 0.796 | 0.700 | 0.748 |

TABLE 35
Zero Shear Viscosity - Quantified as
the Average of the Observed Plateau

| | Zero Shear Viscosity (Pa · s) | | |
|---|---|---|---|
| Sample | Run 1 | Run 2 | Mean |
| 1% Test Article | 322 | 334 | 328 |

TABLE 36
Oscillation Stress Sweep Data

| | Complex Modulus | | | Phase Angle | | |
|---|---|---|---|---|---|---|
| | Plateau (Pa) | | | Plateau (°) | | |
| Sample | Run 1 | Run 2 | Mean | Run 1 | Run 2 | Mean |
| 1% Test Article | 143 | 136 | 140 | 25.9 | 25.5 | 25.7 |

TABLE 37
Oscillation Stress Sweep Additional Data

| | Yield Stress (Pa) | | |
|---|---|---|---|
| Sample | Run 1 | Run 2 | Mean |
| 1% Test Article | 20.6 | 21.8 | 21.2 |

The results demonstrate a clear soft solid structure present under low stress conditions, yielding to non-Newtonian shear thinning flow as the applied stress is increased past a certain yielding stress.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating inflammatory lesions of rosacea, comprising topically administering to the face of an adult human patient in need thereof once per day at or near bedtime a dose of about 1 gram of minocycline topical suspension to treat the inflammatory lesions of rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent comprising (i) from about 2.5% (w/w) to about 10% (w/w) of ethylene-propylene-styrene copolymer having a weight-average molecular weight in the range of from about 150,000 g/mol to about 250,000 g/mol, (ii) from about 0.1% (w/w) to about 2.5% (w/w) of butylene-ethylene-styrene copolymer having a weight-average molecular weight in the range of from about 50,000 g/mol to about 150,000 g/mol, and (iii) at least about 80% (w/w) mineral oil;
wherein particles of minocycline in the topical suspension have a D90 particle size in the range of from about 3 microns to about 6 microns, and the topical suspension comprises about 3% (w/w) minocycline.

2. The method of claim 1, wherein the topical suspension comprises 3% (w/w) minocycline.

3. The method of claim 1, wherein the dose is 1 g of minocycline topical suspension.

4. A method of treating rosacea, comprising topically administering to the face of a patient in need thereof once per day a dose of from about 0.5 g to about 1.3 g of a minocycline topical suspension to treat the rosacea, wherein the minocycline topical suspension comprises:
a) minocycline in a suspended form within the topical suspension;
b) a liquid medium that dissolves less than 5% (w/w) of the minocycline at room temperature after two hours, wherein the liquid medium contains mineral oil; and
c) a polymeric hydrocarbon gelling agent;
wherein particles of minocycline in the topical suspension have a D90 particle size less than 8 microns, and the topical suspension comprises from about 1% (w/w) to about 3% (w/w) minocycline.

5. (canceled)

6. (canceled)

7. (canceled)

8. A method of treating rosacea, comprising topically administering to the face of a patient in need thereof once per day a dose of from about 0.5 g to about 1.3 g of a minocycline topical suspension that comprises from about 1% (w/w) to about 3% (w/w) minocycline to treat the rosacea, wherein the administering results in a minocycline blood plasma AUC_{0-24 hr} less than 0.1 h*μg/mL, a minocycline blood plasma Cₘₐₓ less than 2.45 ng/mL, and any minocycline blood plasma Tₘₐₓ is in the range of 4.5 to 7.5 hours.

9-40. (canceled)

41. The method of claim 1, wherein the minocycline topical suspension comprises from about 66% (w/w) to about 70% (w/w) of the polymeric hydrocarbon gelling agent.

42. (canceled)

43. (canceled)

44. (canceled)

45. The method of claim 41, wherein the minocycline topical suspension comprises from about 27% (w/w) to about 31% (w/w) of the liquid medium that dissolves less than 5% (w/w) of the minocycline at room temperature after two hours, wherein the liquid medium contains mineral oil.

46-53. (canceled)

54. The method of claim 1, wherein the minocycline topical suspension is topically administered to the face of a patient from about 0.5 hours to 1 hour before the patient's bedtime.

55-58. (canceled)

59. The method of claim 1, wherein the patient refrains from topically removing the administered minocycline topical suspension for at least four hours.

60-67. (canceled)

68. The method of claim 1, wherein for a duration of at least 4 weeks the patient receives a dose of minocycline topical suspension each day.

69-72. (canceled)

73. The method of claim 1, wherein the patient has at least 10 inflammatory lesions on their face due to rosacea prior to starting treatment with minocycline topical suspension.

74-77. (canceled)

78. The method of claim 1, wherein the method produces at least a 35% reduction in the number of inflammatory lesions on the patient's face.

79. The method of claim 1, wherein the method produces at least a 45% reduction in the number of inflammatory lesions on the patient's face.

80. (canceled)

81. The method of claim 1, wherein the method produces a reduction in inflammatory lesions in the amount of at least 10 inflammatory lesions on the patient's face.

82. (canceled)

83. (canceled)

84. The method of claim 1, wherein within a population of at least 20 patients the method produces a mean reduction in Erythema Severity Score of at least 0.5.

85. (canceled)

86. The method of claim 1, wherein the method achieves an Erythema Severity Score of absent or mild.

87. (canceled)

88. (canceled)

89. (canceled)

90. The method of claim 78, wherein said reduction occurs within 4 weeks of first receiving minocycline topical suspension.

91. (canceled)

92. The method of claim 79, wherein said reduction occurs within 12 weeks of first receiving minocycline topical suspension.

93. (canceled)

94. The method of claim 78, wherein said reduction lasts for a duration of at least 2 weeks after discontinuing daily administration of the minocycline topical suspension.

95. (canceled)

96. (canceled)

97. (canceled)

98. The method of claim 1, wherein the method produces at least a two-grade improvement in Investigators Global Assessment score.

99. The method of claim 1, wherein the method achieves an improvement in Investigators Global Assessment score resulting in an Investigators Global Assessment score of clear or almost clear.

100. (canceled)

101. (canceled)

102. The method of claim 98, wherein said improvement in Investigators Global Assessment score occurs within 12 weeks of first receiving minocycline topical suspension.

103-108. (canceled)

109. The method of claim 1, wherein any increase in Local Application Skin Reaction Score for erythema, dryness, erosion or edema is no greater than 1.

110. (canceled)

111. (canceled)

112. (canceled)

113. The method of claim 1, wherein the patient's minocycline blood plasma AUC_{0-24 hr} is less than 0.05 h*μg/mL.

114. (canceled)

115. The method of claim 1, wherein the patient's minocycline blood plasma Cₘₐₓ is less than 1.9 ng/mL.

116. (canceled)

117. The method of claim 1, wherein any Tₘₐₓ for minocycline in the patient's blood plasma is in the range of 5 to 7 hours.

118. (canceled)

119. The method of claim 1, wherein the rosacea is papulopustular rosacea.

120-141. (canceled)
